(19) United States
(12) Patent Application Publication
(10) Pub. No.: US 2025/0257309 A1
(43) Pub. Date: Aug. 14, 2025

(54) Systems and Methods for Live Culture Incubation and Monitoring

(71) Applicant: 37degrees, Inc., Geneva, IL (US)

(72) Inventor: Tilak Jain, Geneva, IL (US)

(21) Appl. No.: 19/053,285

(22) Filed: Feb. 13, 2025

Related U.S. Application Data
(60) Provisional application No. 63/553,126, filed on Feb. 13, 2024.

(51) Int. Cl. C12M 1/34; C12M 1/00

(52) U.S. Cl. CPC C12M 41/30; C12M 23/40; C12M 29/06

(57) ABSTRACT

In some examples, a system for cell incubation and imaging includes a first housing around a chamber. The chamber receives a culture. The first housing includes a light-transmissive first imaging access port. The system includes a chamber environment interface that controls a property of an environment within the chamber to maintain the property within a predetermined range (for incubation of the culture). The system includes a chamber environment sensor that measures the property to verify that the property is maintained within the predetermined range. The system includes a sensor within a second housing. The second housing includes a light-transmissive second imaging access port. The sensor captures a representation of the culture through the two imaging access ports while the first housing is detachably coupled to the second housing and while the property of the environment within the chamber is maintained within the predetermined range.

### CROSS-REFERENCE TO RELATED APPLICATIONS

This application claims the benefit of U.S. Provisional Application No. 63/553,126, filed Feb. 13, 2024, and titled “Systems and Methods for Live Culture Incubation and Imaging,” which is hereby incorporated by reference in its entirety and for all purposes.

### BACKGROUND

### Field of the Invention

The present disclosure generally pertains to live culture incubation, perfusion, and/or imaging systems. More specifically, the present disclosure pertains to culture systems that control properties of an environment in a chamber that includes a cell culture to maintain the property within a predetermined range associated with incubation of the cell culture, as well as imaging systems that capture image data of the cell culture over time as the culture systems continue to incubate the cell culture.

### Background

Cells are the fundamental units of life. Cells make up tissues, which then make up organs and organisms. Therefore, observing cells, tissues (e.g., including 3D organoids and/or 3D spheroids) and organs have allowed researchers make significant scientific and economic relevant discoveries in the fields of biology, agriculture, ecology, healthcare, environmental sciences, geology, to name a few. While cells and tissues can be observed in living or ‘fixed’ non-living formats, observing living cells and tissue cultures (mammalian and non-mammalian) have become an essential platform utilized in fundamental cell & tissue biological research, clinical research, drug discovery, disease research, virology, regenerative medicine and/or all domains where a scientist needs to study live cellular and tissue moieties outside the animal organism.

### SUMMARY

Systems and methods are described for cell incubation and/or imaging. In some examples, a system includes a first housing around a chamber. The chamber receives a culture. The first housing includes a first imaging access port that is light-transmissive. The system includes at least one chamber environment interface that controls a property of an environment within the chamber to maintain the property within a predetermined range. Therein the predetermined range is associated with incubation of the culture. The system includes at least one chamber environment sensor that measures the property of the environment within the chamber to verify that the property is maintained within the predetermined range. The system includes at least one sensor within a second housing. The second housing includes a second imaging access port that is light-transmissive. The at least one sensor captures a representation of the culture within the chamber through the first imaging access port and the second imaging access port while the first housing is detachably coupled to the second housing and while the property of the environment within the chamber is maintained within the predetermined range.

In some aspects, the techniques described herein relate to a system for culture processing. The system includes a first housing around a chamber. The chamber receives a culture. The first housing includes a first imaging access port that is light-transmissive. The system includes at least one chamber environment interface that controls a property of an environment within the chamber to maintain the property within a predetermined range. The predetermined range is associated with incubation of the culture. The system includes at least one chamber environment sensor that measures the property of the environment within the chamber to verify that the property is maintained within the predetermined range. The system includes at least one sensor within a second housing. The second housing includes a second imaging access port that is light-transmissive. The at least one sensor captures a representation of the culture within the chamber through the first imaging access port and the second imaging access port while the first housing is detachably coupled to the second housing and while the property of the environment within the chamber is maintained within the predetermined range.

In some aspects, the techniques described herein relate to a method of culture processing. The method includes receiving a culture into a chamber within a first housing. The first housing includes a first imaging access port that is light-transmissive. The method includes controlling a property of an environment within the chamber using at least one chamber environment interface to maintain the property within a predetermined range. The predetermined range is associated with incubation of the culture. The method includes measuring the property of the environment within the chamber using at least one chamber environment sensor to verify that the property is maintained within the predetermined range. The method includes capturing a representation of the culture within the chamber using at least one sensor through the first imaging access port and a second imaging access port while the first housing is detachably coupled to a second housing and while the property of the environment within the chamber is maintained within the predetermined range. The second housing includes the at least one sensor and the second imaging access port that is light-transmissive.

In some aspects, the techniques described herein relate to a non-transitory computer readable storage medium having embodied thereon a program, wherein the program is executable by a processor to perform a method of culture processing. The method includes receiving a culture into a chamber within a first housing. The first housing includes a first imaging access port that is light-transmissive. The method includes controlling a property of an environment within the chamber using at least one chamber environment interface to maintain the property within a predetermined range. The predetermined range is associated with incubation of the culture. The method includes measuring the property of the environment within the chamber using at least one chamber environment sensor to verify that the property is maintained within the predetermined range. The method includes capturing a representation of the culture within the chamber using at least one sensor through the first imaging access port and a second imaging access port while the first housing is detachably coupled to a second housing and while the property of the environment within the chamber is maintained within the predetermined range. The second housing includes the at least one sensor and the second imaging access port that is light-transmissive.

In some aspects, the techniques described herein relate to a system for culture processing. The system includes means for receiving a culture into a chamber within a first housing. The first housing includes a first imaging access port that is light-transmissive. The system includes means for controlling a property of an environment within the chamber using at least one chamber environment interface to maintain the property within a predetermined range. The predetermined range is associated with incubation of the culture. The system includes means for measuring the property of the environment within the chamber using at least one chamber environment sensor to verify that the property is maintained within the predetermined range. The system includes means for capturing a representation of the culture within the chamber using at least one sensor through the first imaging access port and a second imaging access port while the first housing is detachably coupled to a second housing and while the property of the environment within the chamber is maintained within the predetermined range. The second housing includes the at least one sensor and the second imaging access port that is light-transmissive.

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

### BRIEF DESCRIPTION OF THE DRAWINGS

Illustrative embodiments of the present application are described in detail below with reference to the following figures:

FIG. 1 is a cutaway architecture diagram illustrating an incubator system, according to some examples;

FIG. 2 is a cutaway architecture diagram illustrating an imaging system, according to some examples;

FIG. 3 is a cutaway architecture diagram illustrating the incubator system and the imaging system combined to form an incubation and imaging system, according to some examples;

FIG. 4 is a flow diagram illustrating an exemplary process for performing long-term cell or tissue culture incubation using the incubator system, according to some examples;

FIG. 5 is a flow diagram illustrating an exemplary process for performing long-term cell or tissue culture imaging using the incubation and imaging system, according to some examples;

FIG. 6 includes exploded perspective views of the incubation and imaging system, according to some examples;

FIG. 7 is a perspective view of the bottom module of the incubator system, according to some examples;

FIG. 8 is a cutaway architecture diagram illustrating the incubation and imaging system, according to some examples;

FIG. 9 includes a front view of the incubation and imaging system, a perspective view of the incubation and imaging system, and a side view diagram of a gas cartridge, according to some examples;

FIG. 10 includes internal views of internal components of the incubation and imaging system, according to some examples;

FIG. 11 includes internal views of internal components of the incubation and imaging system, according to some examples;

FIG. 12 is a user interface diagram illustrating a dashboard interface for visualizing data from an incubation and imaging system, according to some examples;

FIG. 13 includes block diagrams illustrating two approaches to regulate and inject controlled quantities of gas into the incubated chamber, according to some examples;

FIG. 14 is a perspective view of an incubation and imaging system in which the imaging system is on top of the incubator system, and various internal components are visible, according to some examples;

FIG. 15 is a perspective view of a cell culture setup to perform in-vitro neuronal recordings on microelectrode arrays, according to some examples;

FIG. 16 is a conceptual diagram illustrating various features of the incubation and imaging system, according to some examples;

FIG. 17 is an example of a phase contrast image (modified to be viewable in black and white) of MCF10A wild type cells successfully cultured in the incubator system and imaged, according to some examples;

FIG. 18 is a conceptual illustration of an incubation and imaging system performing multimodal neuronal stimulation and microscopy, according to some examples;

FIG. 19 is a perspective diagram illustrating an imaging system, according to some examples;

FIG. 20 is a cross-section diagram illustrating an imaging system, according to some examples;

FIG. 21 is a perspective diagram illustrating an incubation and imaging system, according to some examples;

FIG. 22 is a block diagram illustrating an example of a machine learning system for training, use of, and/or updating of one or more machine learning model(s) that are used to generate analysis and/or recommendation(s), in accordance with some examples;

FIG. 23 is a flow diagram illustrating a process for cell incubation and imaging, in accordance with some examples.

FIG. 24 is a block diagram of an exemplary computing device that may be used to implement some aspects of the technology.

### DETAILED DESCRIPTION

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter. Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

Systems and methods are disclosed for cell culture incubation, imaging, and management. In some examples, a system for live cell and tissue culture imaging includes of an incubation subsystem and an imaging subsystem. The incubation subsystem allows for long-term live cell or tissue culture incubation at controlled environmental conditions, for instance including temperature, relative humidity, and gas concentrations. The imaging subsystem allows for long-term live culture imaging and subsequent analysis using on-system computing and online cloud-based computing modalities. The subsystems are battery-powered and light-weight, allowing for portability of live cultures between user locations. The user loads live cultures into the incubator subsystem and can physically, optically and electrically integrate it with the imaging subsystem to commence live culture analysis. The imaging subsystem, which is anti-vibration stabilized, performs rough and fine auto-focusing, while initiating time-lapse camera-based data acquisition. On-board analysis of raw camera data is performed on high-speed computing subsystems, allowing for high frame-rate analysis. Online cloud-connectivity pipelines provide for remote analytics of incubation and imaging functions.

Sustaining, manipulating and analyzing live cells and tissues, allows the researcher to gain rich insights over longer time-frames and make discoveries. In accordance with some embodiments of the present disclosure, a modular cell and tissue incubator subsystem is disclosed herein that utilizes minimal bench-space and is cost-effective, such that individual researcher can have ‘personal’ units on their respective lab bench, thereby reducing potential for contamination between researchers. Integration of simple and safe miniature gas supply sources (cartridges) inside the incubator subsystems itself thereby relieves the researchers from having to coordinate large gas tank hookups to walls with facilities support, also increasing general lab safety. In some embodiments, the incubator subsystem is battery powered and operable at low DC voltages. Chamber volumes are minimized to satisfy culture dish sizes and lowering gas supply cost. In some examples, an imaging system is integrated with the incubator system, so that the cell cultures can be imaged as the incubator continues to incubate the cell cultures.

Visualizing cells and tissues with high-magnification time-lapse video is especially valuable in dynamic studies such as, but not limited to, drug-effects on mitigating cancer progression, neuronal degeneration, metabolic syndromes, viral progression and immunological responses. Along with rapid advancements in areas such as gene editing, stem-cell and tissue engineering, cell and gene therapy, laboratories are in need to efficiently sustain, monitor and analyze precious cells and tissues. In some examples, systems and methods described herein may use image analysis pipelines (e.g., CellProfiler, ImageJ). In some examples, systems and methods described herein may use ultra-fast parallel computing on graphical processing units (GPUs), image analysis using machine learning and/or artificial intelligence, and/or other techniques, for rich information gathering and analysis from biological image-based data.

In some evolving healthcare scenarios (such as Advanced Therapy Medicinal Products ATMP), cell and tissue cultures need also to be transported from the patient sites and/or bio-repositories, to local and global collaborator sites. Alternatively, laboratories working jointly on unique problems, can want to share their cell lines and tissues of interest within departments, universities, divisions and/or organizations located in different states and perhaps even countries. However, transportation of cells and/or tissues between labs (and especially when distances are greater than a few miles), is traditionally conducted cryogenically in liquid nitrogen and/or on dry ice, both of which are logistically challenging and can hamper the viability of the cells and/or tissues, given the nature of the freeze-thaw cycle.

In some examples, an exemplary researcher workflow for a scientific lab can include extracting cells or tissues from a diseased organism, a normal organism (e.g., non-diseased), a biological repository, another laboratory, or a combination thereof. The workflow can include placing the cells or tissues in one or more petri dishes (in any of a set of different physical formats), with an appropriate amount of cell culture media (comprising of nutrients, serum, pH indicators and buffers) and in some cases with experimental reagents of interest. The workflow can include placing the petri-dish in an incubation environment, which maintains control of temperature (e.g., within a range around 37 degrees C. for mammalian cells, but can vary), relative humidity (e.g., >60%) and gas composition (e.g., a specified distribution of Carbon dioxide, Nitrogen, and/or Oxygen at respective concentration percentages) to specified control points. The workflow can include observing the cells or tissues over time via a modality such as microscopy and/or, in some cases, biochemical or neural recording. These observations can last a few seconds to several weeks or months (in case of some cell types such as primary neurons and stem cells). The workflow can include analyzing every recorded data (images and/or other biochemical, molecular and/or physiological signals). The workflow can include testing scientific hypothesis and/or making discoveries relevant to the experimentation.

There are several challenges in scientific lab research workflows. The challenges in research workflows include that cell and/or tissue culture incubators tend to be shared by multiple researchers, given their limited availability due to high price and space constraints. This can lead to microbial contamination between researchers (e.g., between experiments), potentially ruining valuable experimentation. The challenges in research workflows include that traditional cell and tissue culture incubators are bulky (e.g., often the size of small refrigerator or larger), therefore not portable, often operated at high 110/220 AC voltage. Generally, if a researcher wants to transport their cell and/or tissue cultures, the researcher has to remove the cultures from the incubator and quickly move the cultures to the destination. Cells and/or tissues can be very sensitive to environmental conditions, and if transportation is prolonged, can lead to loss of experimentation and/or false results. Sharing of cells and tissues in liquid nitrogen and/or on dry ice is logistically challenging. The researcher has to have access to liquid nitrogen and/or dry ice to transport cultures safely, which is not readily available in many countries. They will always need to disrupt the cells and tissues native environment (such as breaking them away from their adherent surface and/or replacing the culture media with additional cryo-protectants). At the destination site too, there are requirements for the receiving party to handle the shipment safely, thaw the cells and tissues to culture the cells and tissues back, all of which can lead to loss of viable cells.

Furthermore, given a requirement to control gas composition and percentages at specified levels, cell and tissue incubators typically depend on an external tank(s) of gas, which can cause additional challenges in research workflow. This supply line is piped into the incubator and attached via pneumatic couplers. Given the large chamber volumes of incubator (e.g., >1 cubic ft), these supply tanks are typically large (e.g., greater than 5 pounds, in some examples 50 pounds) and are securely affixed to the walls next to the incubator with chains. This severely limits the portability of cells and tissues in a controlled environment, it also requires regular facilities support to install and monitor any safety concerns. Additionally, given the large volumes of the incubation chamber, gas requirements increase usage costs.

Another challenge in research workflows is that traditional cell and tissue incubators are not equipped with imaging components (e.g., microscopy components). Thus, a researcher using a traditional incubator has to remove their cells and tissue cultures from the incubator and quickly load the cells onto a nearby microscope to take images and/or videos, before rapidly bringing the cells back into the incubated environment. This severely limits how, and for how long, the researcher can do long-term microscopy without disturbing the cell and tissue environment. For instance, in some cases, the researcher is limited to imaging the cells for a few hours or (on rare occasions) days, but cannot image the cells for longer periods of time (e.g., weeks or months) without disturbing the cell and tissue environment, potentially damaging or even killing the cells. In some examples, a microscope may be encased in a ‘case’ incubator positioned around a microscope, a ‘stage’ incubator may be placed on a microscope, and/or the microscope itself may be placed within a large incubator. However, without further integrations (such as those discussed herein), such combinations can limit compatible devices, limit available resources, and do not provide systematic integrations between the incubator and the microscope to control the culture conditions and microscopy conditions, leading to inefficient workflows. In some examples, for instance, image focus losses can be caused by spatial temporal temperature shifts between the culture and optical system environments, in some cases requiring special objective lens heaters to compensate for the z-focus drifts. Such combinations can also remain dependent on large fixed gas tanks and/or bulky equipment that is not portable.

Even if a researcher is able to perform long-term cell and/or tissue microscopy and acquire digital assets, traditional instruments (e.g., microscopes) are not able to, by themselves, process the images and/or videos (e.g., high-resolution images at high frame rates) that they capture, and thus, a user may have to store raw data files on isolated hard-disks-another challenge of research workflows. Even if the financial issue of storage costs is overcome, a major hurdle of analyzing massive data volumes and transfer to off-system compute environment remains. For example, at 25 fps and a moderate 5M pixel resolution, with low 8-bit image depth, a single day of imaging can yield >10 TB of raw data. These storage amounts climb exponentially with higher number of experimental days, higher camera resolutions, richer and more color depth, and/or greater frame rates (e.g., which may be required to capture certain cellular events that happen in a millisecond or less). Thus, storage and/or analysis of data can quickly become a bottleneck in experimentation, can greatly reduce throughput of an experiment, speed of analysis, and/or flexibility of research hardware. In some cases, this issue can push a researcher to reduce image resolution and/or frame rate below what they would like to use, which may cause the researcher to fail to capture important events.

In some cases, research workflows involve a need to interface with the cell cultures while the cell cultures are under incubation. These interfaces can include, but are not limited to, the need to perfuse the cell and tissue culture media regularly, add or remove manipulation reagents for controlled experimental perturbation and/or for creating specific imaging conditions, perform neuronal recordings using micro-electrode arrays embedded within the culture dishes. These interfaces are therefore fluidic, electrical, optical and/or pneumatics based. In some examples, the interfaces are co-located next to the incubators, but not integrated, which further restricts portability. In some examples, interface lines can be awkwardly positioned, restricting access into the incubator and/or creating sealing issues. All of these can be issues with traditional research workflows.

As noted above, in some examples, challenges in research workflows can include sharing cell and tissue culture incubators and imaging systems, which can lead to contaminated cell cultures. Challenges can include the fact that traditional incubators, gas tanks, and/or microscopes are large and bulky and are generally not compatible with one another, and thus each take up precious lab space and need facilities support (e.g., for large gas tank delivery and high voltage AC power hook-up), limiting what experiments users can run and increasing utility costs. Challenges in research workflows can include traditional incubators, gas tanks, and/or microscopes not being compatible with one another and not being portable, meaning that cells generally must be moved frequently between devices and sometimes between buildings or longer distances (e.g., to a different country). Cell and tissue cultures often get destroyed during inter-lab transfer to core microscopy facilities. Inter-lab cell and tissue culture sharing, particularly across geographic regions, is a logistical problem, with freezing, thawing, and/or other travel-related disturbances leading to loss of viability of cells. Challenges in research workflows can include that traditional solutions for long-term live cell culture microscopy are not integrated with traditional incubator solutions, generally requiring a user to remove cells from the incubator to allow the cells to be imaged. Challenges in research workflows can include that traditional microscopes do not perform real-time in-system image analysis, instead requiring enormous amounts of data to be stored and/or analyzed on a different device, leading to large data bottlenecks. Challenges in research workflows can include that traditional systems can limit external world interactions and/or manipulations of cell and tissue cultures via fluidic, optical, mechanical and electrical stimuli, for instance requiring cells to be removed from an incubator for any interactions and/or manipulations.

Systems and methods described herein overcome these challenges to empower the usage of long-term live cell and tissue culture incubation and analysis into high-impact areas such as research efforts in academia, government, industry and within service-based CRO units, and in science education in school, college and university settings. The systems and methods described herein augment research and discovery efforts, and empower the strengthening of education domestically and internationally. For instance, the systems and methods described herein allow for cell and tissue cultures to be readily analyzed within laboratories, and for cell and tissue cultures to be shared in their native environment across geographical borders (e.g., maintained in an incubation environment during travel), allowing for greater outreach and collaboration.

The systems and methods described herein further provide for reduced opening and closing of incubator doors, which change environmental conditions impacting cell cultures within minutes and cause variability of scientific results. For instance, integration of an imaging system with an incubator means that cell cultures can remain in the incubator while the imaging module captures images, videos, and/or other sensor data from the cell cultures. Real-time monitoring of the cell cultures further provides enhanced reliability and flexibility, allowing the system to capture images and/or videos (and/or other representations) of rare occurrences (e.g., by allowing for longer-term imaging), allowing the system to continue incubating the cell culture (e.g., keeping the cell culture alive) and monitoring the cell culture even without any human interaction (e.g., allowing users to perform other tasks or take a break, for instance for a holiday) or with minimal human interaction (e.g., to change a setting remotely using user device coupling to the incubator and/or imaging system over a network and/or via a cloud service).

Systems and methods are described for cell incubation and/or imaging. In some examples, a system includes a first housing around a chamber. The chamber receives a culture (e.g., a cell culture). The first housing includes a first imaging access port that is light-transmissive (e.g., is transparent, translucent, and/or includes a hole or aperture in the first housing). The system includes at least one chamber environment interface that controls a property (e.g., temperature, humidity, pressure, pH, fluidic flow rate, orientation, level of illumination, gas concentration, liquid concentration, reagent concentration, etc.) of an environment within the chamber to maintain the property within a predetermined range. Therein the predetermined range is associated with incubation of the culture. The system includes at least one chamber environment sensor that measures the property of the environment within the chamber to verify that the property is maintained within the predetermined range. The system includes at least one sensor within a second housing. The second housing includes a second imaging access port that is light-transmissive (e.g., is transparent, translucent, and/or includes a hole or aperture in the second housing). The at least one sensor captures a representation of the culture within the chamber through the first imaging access port and the second imaging access port while the first housing is detachably coupled to the second housing (e.g., via latches, magnets, pins into recesses, screws, clips, slide rails, elastic, seals, hook and loop fasteners, stud fasteners, other types of fasteners, or combinations thereof) and while the property of the environment within the chamber is maintained within the predetermined range (e.g., by the chamber environment interface).

Cell incubation and/or imaging systems can include various improvements, various limitations, or combinations thereof. In a first illustrative example, a small incubation unit (e.g., 22×22×22 cm, weighing 3.6 kg) can be placed inside a microscopy unit. However, in some cases, such a system can still require large CO2-fillable tanks, can be operated at high AC voltage of 220V (not battery powered), can lack on-board sensors and real-time sensor connectivity to track and monitor incubation performance, can lack incorporation of on-system live frame computing, and/or can lack a way to add additional interfaces (electrical, perfusion, optical and pneumatic). In a second illustrative example, a small live cell culture incubator device (e.g., the size of a travel suitcase) can allow for improved travel, but can still be limited by lacking any imaging modality and/or lacking means to actively supply gas (e.g., depending on an initial equilibration of carbon dioxide CO2 using a larger incubator), reducing transport time capability given environmental degradation of the desired gas composition. A small incubator device, on its own, still lacks integration of incubation with imaging modalities and/or image-based data analytic capabilities while the cells are in an incubated environment. In a third illustrative example, a small incubator device can control for gases (e.g., carbon dioxide (CO2) and/or nitrogen (N2)) (e.g., for air composition), with CO2 and N2 gases still supplied from externally placed tanks and not integrated within the system, limiting portability and effective travel range. In some examples, an incubator may rely on dry ice, which can sublimate over time and further limit portability and effective travel range. In a fourth illustrative example, a microscope can be placed in a large incubator (e.g., with large wall-mounted gas tanks), however such a combination is not portable.

Systems and methods described herein provide advanced solution(s) for cell & tissue culture incubation, imaging, analysis and inter-lab sharing of cultures in their native environment (e.g., in an incubation environment). Systems and methods described herein allow for easy-to-use, inexpensive, portable, lab-bench compatible, modular, cloud-connected for real-time and longitudinal analytics and on-board rich computing resources coupled to high-magnification and/or high-frame rate imaging modalities.

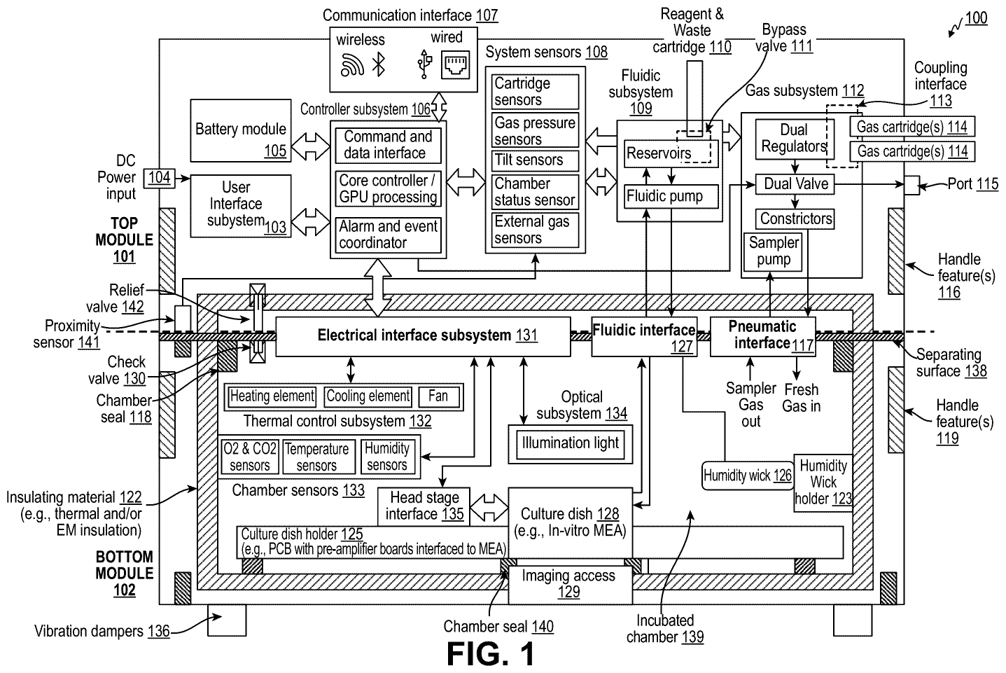

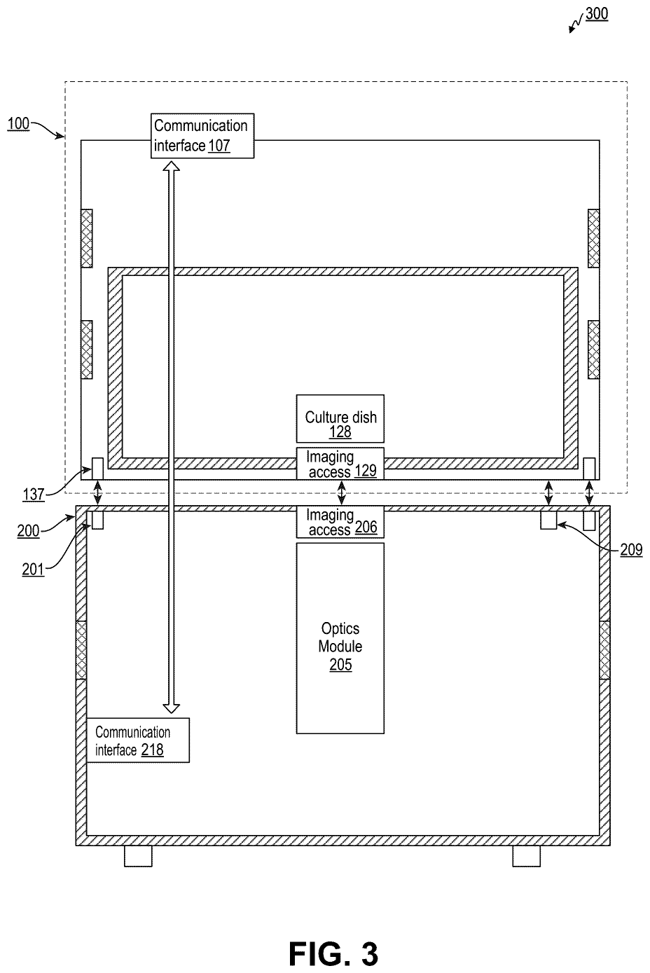

FIG. 1 is a cutaway architecture diagram illustrating an incubator system 100. The incubator system 100 can be referred to as a portable cell and tissue incubator system, or an incubation subsystem. In some examples, the incubator system 100 can be referred to as an incubator subsystem or an incubation subsystem, for instance when the incubator system 100 is used as a subsystem of a combined incubation and imaging system (e.g., incubation and imaging system 300 of FIG. 3).

FIG. 1 shows an embodiment of the major subsystems, components and interfaces of the incubator system 100. In the presented embodiment, the incubator system 100 includes a top module 101 and a bottom module 102, which can come together to close or be separated to open the incubated chamber 139. The top module 101 and bottom module 102 are physically separated by a surface 138 and a chamber seal 118 (e.g., with a reversible sealing mechanism), both of which can reside within the top module 101 (but can also reside with the bottom module 102). The separation surface 138 allows for physical interfaces to occur between the top module 101 and bottom modules 102. The separation surface 138, via the aid of the chamber seal 118 (e.g., the reversible sealing mechanism), allows for the user to open and close the incubator system 100 to gain access to the incubated chamber 139, using handle features 116 provided on the top module 101. A proximity sensor 141 allows for monitoring the separated or closed state of the top module 101 from the bottom module 102. In some examples, the incubator system 100 includes anti-vibration legs 136, which reduce external vibrations from entering the subsystem and improving the performance and operational reliability of the subsystem. In some examples, the incubator system 100 includes alignment features 137, such that the subsystem can be physically docked with other subsystems of varying type (such as the imaging system 200). Such alignment features can be readily implemented using pin and socket mechanical features and can also be magnetic in nature for increased hold. In some examples, alignment and/or docking can be implemented with snap-and-release mechanisms or with electronic activated latches.

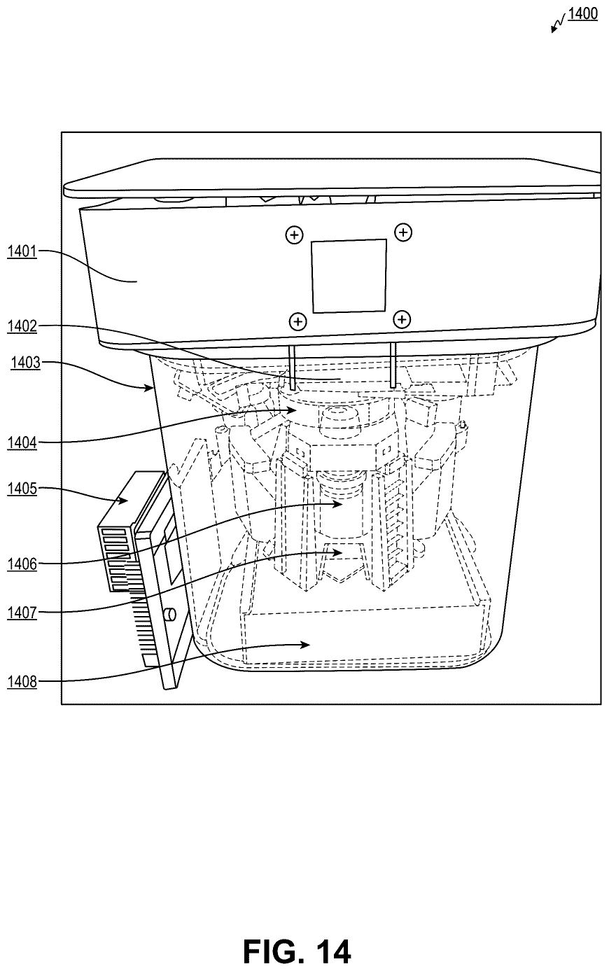

In some examples, the bottom module includes an imaging access port 129 (e.g., microscopy access port), to allow for a transparent path for microscopy of the cell and tissue culture dish 128, when the incubator system 100 is docked on top of an imaging system 200. The imaging access port 129 can be referred to as a microscopy access port. In this embodiment, there is a provision to maintain a chamber seal 140 (using but not limited to sealing gaskets, silicone o-rings or sealing gels) between the culture dish 128 and the incubated environment in the incubated chamber 139, such that only the bottom of the culture dish is exposed to the outside environment when the imaging access port 129 is opened. In some examples, the imaging access port 129 is not provided (e.g., missing), for instance when applications do not require microscopy or if the microscope is provided inside the incubated chamber 139 (as described in FIG. 14). The entirety of the incubator system 100 is portable and handle features 119 provided on the bottom module 102, further aid in doing so. In some examples, a handle strap may be included on the top module 101 and/or bottom module 102, with a mechanical locking feature to keep both these subsystems together while transportation. To thermally isolate the incubated chamber 139, all chamber exposed surfaces of the top module 101 and bottom module 102, are lined with an insulating material 122. The insulated material can be made of, but not limited to, materials such as fiberglass, foam and ceramic fillings or with specially designed 3D printed materials with insulation properties, or combinations thereof. While shown in FIG. 1 is an embodiment where the top module 101 is placed on top of the bottom module 102, alternative embodiments may have the subsystems placed side by side or in reverse order. Alternatively, also the incubated chamber may have a separate and reversibly sealed access door to load and unload the culture dish 128, without having to separate the top and bottom modules. Such an additional access door may provide further reduction of environmental changes during culture dish exchanges.

The culture dish 128 may include a cell culture, which can include, in some examples, one or more cells, tissues, organoids (e.g., 3D organoids), spheroids (e.g., 3D spheroids, organs, portions of any of the previously-listed materials, or combinations thereof. In some examples, the culture dish 128 may include media that the cell culture is grown within and/or perfused into.

In some examples, the physical interfaces between the top module 101 and bottom module 102 include an electrical interface subsystem 131, a fluidic interface subsystem 127 and a pneumatic interface subsystem 117, all of which reside on the separation surface 138. In some examples, the fluidic interface subsystem 127 may not be necessary; if no fluid exchange is needed for the incubator subsystem application. The interfaces are air-tight sealed best possible using off-the-shelf gaskets, adhesives and/or washers (not shown) through the separation surface 138, to ensure that the incubated chamber 139 remains isolated from the external environment.

In some examples, the electrical interface subsystem 131, the pneumatic interface subsystem 117, and/or the fluidic interface subsystem 127 can reside entirely on (and/or in) the bottom module 102, and can directly provide access to the incubated chamber 139. In such examples, external systems can interface with the incubator system 100 via reversible fluidic, electrical and/or pneumatic connectors.

In some examples, the separation surface 138 includes a check valve 130 and/or a relief valve 142. In some examples, the incubator system 100 can use the check valve 130 and/or the relief valve 142 to release gas(es) (e.g., air) from the interior of the incubated chamber 139 in the bottom module 102 (e.g., to the top module 101, to the exterior of the incubator system 100, and/or otherwise to the exterior of the incubated chamber 139). In some examples, the check valve 130 and/or the relief valve 142 allow for air-escape when the top module 101 and bottom module 102 are closed together and sealed via the chamber seal 118 (e.g., the reversible sealing mechanism). The check valve 130 and/or the relief valve 142 allow for balancing the positive or negative pressure in the incubated chamber 139 (e.g., to be within a threshold range of atmospheric pressure), caused by either injection or removal of material into or out of the incubated chamber 139. The electrical interface subsystem 131 includes electrical connectors for digital, analog and power signals to and from the thermal control subsystem 132, chamber sensor subsystem 133, optical subsystem 134 and a head stage interface 135, that are present within the incubated chamber 139. In some examples, the fluidic interface subsystem 127 includes fluidic ports to and from the culture dish 128 and the humidity wick 126 (or humidity stick). The humidity wick 126 is made of water retaining material which allows for evaporation and increasing the humidity inside the incubated chamber, such as but not limited to, a porous wick, sponge or towel and positioned at a designated humidity wick holder 123 (or humidity stick holder).

In some examples, a fluidic port may be used to inject mist into the chamber 139 to increase humidity, which can be accomplished by pressurizing water through fine nozzles. In some instances, instead of a humidity wick, water can be dropped onto a small ultrasonic element that in turn then creates mist inside the chamber.

In some examples, the fluidic interface subsystem 127 may be present in the incubator system 100. In some examples, the fluidic interface subsystem 127 may be missing in the incubator system 100, for instance if the application does not require reagent exchanges, and/or if humidity control is achieved by simply wetting the humidity stick, prior to closing the chamber, without active delivery of water to the stick. The pneumatic interface subsystem 117 includes through-panel mechanical ports for gas input or sampling out of the incubated chamber 139. In some examples, the pneumatic interface subsystem 117 include additional ports to allow for calibrated gas to be purged into the incubated chamber 139 for gas sensor calibration. While a single electrical interface, fluidic interface and pneumatic interface is depicted in FIG. 1, the interfaces themselves may be partitioned and spatially distributed across the separating surface 138 to aid for compact design.

In some examples, the thermal control subsystem 132 is placed within the incubated chamber 139 includes a heating element, cooling element and a circulating effector such as a convective fan or blower. In some examples, a cooling element may not be necessary to arrive at temperature set-points and only a heating element is provided. Heating and cooling elements may be implemented using a wide variety of electronic components, including but not limited to, peltier-based thermoelectric heater-cooling, resistive heaters and ceramic heating. There may be one or several heating elements, cooling elements and/or convective elements placed inside the incubated chamber to accomplish uniform heat distribution. In some examples, the thermal control subsystem may be placed outside the incubated chamber 139 and in the top module 101, to reduce electronics within the incubated chamber. In such an arrangement, a blower may push heated or cooled air into the chamber to arrive at the temperature set-point.

In some examples, the chamber sensor subsystem 133 is placed within the incubated chamber 139 includes gas sensors, temperature sensors and humidity sensors. In some examples, a Carbon dioxide and/or an Oxygen gas sensor is/are configured. In some examples, both a Carbon dioxide and an Oxygen gas sensor are configured. In some examples, additional gas sensors (such as gas sensor(s) for Nitrogen and/or other gases) may also be included. In some examples, single temperature and relative humidity sensors are configured. In some examples, multiple temperature and relative humidity sensors are implemented to sense these parameters at different spatial points of the incubated chamber 139. As shown in FIG. 1, an illustrative example is for the chamber sensor subsystem 133 to be placed within the incubated environment as to sense these environmental parameters directly. However, in some examples, the chamber sensor subsystem 133 entirely or some of its component may be placed outside the incubated chamber 139 and in the top module 101. In such an arrangement, a sample of environmental air can be pulled outside using a sampling pump and onto the sensors.

In some examples, the optical subsystem 134 is placed with the incubated chamber 139, and comprising of an illumination source for microscopy that is positioned above the culture dish 128. Such an illumination source can be implemented using, but not limited to, an array of light-emitting diodes in a rectangular or ring orientation, and operated in a continuous ON mode and/or in a pulsed-mode. When activated, the illumination sourcing (e.g., illumination source 204), which may be coupled to additional optical elements such as condenser lenses and filters, floods the culture samples for bright-field imaging, dark-field imaging, fluorescence imaging, and/or phase-contrast imaging (e.g., in conjunction with an imaging system 200). In some examples, the optical subsystem may include both the illumination source and optical lenses to confer upright microscopy of the culture samples placed in the culture dish 128.

In some examples, a culture dish holder 125 or a plurality of such holders stacked onto each other, can be placed inside the incubated chamber 139. Each culture dish holder 125 can have designated locations to hold one or a plurality of culture dishes 128. In an illustrative example, both the culture dish holder 125 and the culture dish 128 can be easily unloaded by the user outside the incubated chamber 139. However, alternative embodiments may fix the culture dish holder 125 within the incubated chamber, with the user having to load and unload the culture dish into the holder. In some examples, the culture dish holder 125 may also carry the humidity wick holder 123, which can hold a humidity wick 126. In some examples, the bottom module 102 (e.g., the incubated chamber 139) can include other water storing components that allow for passive or active (such as the ultrasonic mist generation component described above) release and/or increase of humidity in the incubated chamber 139, such as humidifiers with water talks or reservoirs. In some examples, the bottom module 102 (e.g., the incubated chamber 139) can include a dehumidifier to absorb or otherwise reduce humidity in the incubated chamber 139. The culture dish holder can be placed on an anti-vibration component (e.g., anti-vibration legs 136), to prevent the culture dish from experiencing external vibrations and movement impacts when the incubator system 100 is transported around by the user.

In some examples, the culture dish is a standard round petri-dish. In some examples, the culture dish 128 can take different formats used in laboratories such as well-plates (with multiple culture wells), tissue culture flasks, perfusion cassettes, culture dishes with thin cover-glass at bottom for high-magnification imaging, microelectrode array dishes or microfluidic chips. If the application calls for fluidic exchange, the culture dish 128 may have input and output fluidic ports with fluidic lines coupled to the fluidic interface subsystem 127, via plastic tubing, silicone tubing, and/or metal tubing. The culture dish 128 includes cells or tissues of the user's choice and with the added media, suited for the application.

In some examples, the head stage interface 135 (which may be referred to as an additional electrical interface) allows for expanding the functionality provided by the incubator system 100. One such functionality is the ability to perform neuronal activity recordings using micro-electrode arrays (MEA). In such a scheme, the head stage interface 135 can connect to the MEA-based culture dish and provide the electrical recording connectivity to the electrical interface subsystem 131. Other such schemes can include monitoring ion-channel signals using pH electrode arrays embedded within the culture dish 128. In another embodiment, the additional electronic interface may connect to a specially designed culture dish holder that has an array of ultraviolet lights that are used to illuminate the incubated chamber and provide a means for contamination control. In yet another embodiment, the head stage interface 135 may connect to a special culture dish holder that has a micro-bacterial reagent mister to provide the means for contamination control. In yet another embodiment, the head stage interface 135 may connect to a special culture dish holder that has additional heating elements to induce a high-temperature sterilization cycle. In yet other embodiments, the head stage interface 135 may connect to a special culture dish holder that has additional sensors to expand the functionality of the chamber sensor subsystem 133.

In some examples, the top module 101 includes a DC power input 104, a battery module 105, a user interface subsystem 103, a Controller subsystem, a System sensor subsystem 108, a fluidic subsystem 109 and a Gas subsystem 112. In some examples, a fluidic subsystem 109 may not be included in the incubator system 100, for instance if an application (for which the incubator system 100 is to be used) does not required active reagent exchanges.

In some examples, the DC power input 104 allows for plugging into an external supply to power the incubator system 100 and charge the battery module 105. When the DC power input is unplugged, the incubator system 100 continues to be powered by the battery module 105. The battery subsystem can be made of rechargeable material, such as but not limited to, Lithium-ion, Lithium-poly, Nickel-Metal Hydride or Nickel-Cadmium. In some examples, the unit may be powered by a single-use battery. In some examples, the battery module 105 powers the controller subsystem 106, which then distributes power to the remaining subsystems in the top module 101 and electronic components in the incubated chamber 139, via the Electrical interface subsystem 131. In some examples, the battery subsystem may be connected to a power-distribution subsystem (not illustrated in FIG. 1), which in turn distributes the power.

In some examples, the controller subsystem 106 includes a micro-processor or micro-controller board that is pre-loaded with a firmware program. The firmware program comprises a command and data interface routine for communicating with the external world via the communication interface 107. This routine accepts commands, such as but not limited to, set-points, calibration curves and configuration parameters to allow the user to control the incubator system 100. The routine also transmits sensor data, events, warnings and logs to the user for monitoring incubator system 100 performances, functionality and reliability. The communication interface 107 provides a wireless (via protocols such as but not limited to Wifi, Bluetooth, Cellular) and/or wired (via protocols such as but not limited to USB, Ethernet, serial port, I2C, MOSI). The controller subsystem 106 also includes a core controller routine, which accepts the set-point information provided by the user, and along with chamber sensor subsystem 133 data, performs a close-loop feedback control to achieve environmental conditions inside the incubated chamber 139. The controller subsystem 106 also includes an Alarm and event coordinator routine, to read sensor information from the system sensor subsystem 108 and warn the user of the state of the incubator subsystem. In some examples, the controller subsystem 106 may also include additional routines, such as adaptive machine learning for better control accuracy and/or storage capacity to allow temporary data storage in case of lost communications.

In some examples, a machine learning algorithm and/or model can monitor incubated chamber 139 temperature and change heating parameters in order to reduce the time it takes to arrive stably within a specified set-point. Inputs into the machine learning algorithm and/or model can include external environment temperature, past chamber open-close event history, heater component and fan life, relative humidity conditions, chamber gas concentration conditions, additional user provided information such as culture dish 128 type, size and/or count, current state and/or history of fluid exchanges via the fluid interface subsystem 127, optical subsystem 134 current state (e.g., image(s), video(s), and/or current capture settings) and/or history (e.g., previously captured image(s), video(s), and/or previous capture settings), current state and/or history of gas exchanges via the pneumatic interface subsystem 117, current state and/or history of any addition electrical interfaces 135 present in the chamber. In some examples, a machine learning algorithm and/or model can predict the timing of empty/full state of the gas cartridge 114 and/or the reagent & waste cartridge 110, by utilizing current and/or history of valve operation described in the gas subsystem 112, pressure conditions recorded by the system sensor subsystem 108, current state and/or history of fluid exchanges via the fluid interface subsystem 127 and/or chamber opening/closing event history. In some examples, a machine learning algorithm and/or model can predict battery module 105 charge capacities, the implementation of which would further aid the user to assess and/or plan available culture transport time without DC power input 104. In some examples, the machine learning algorithm and/or model may utilize, as inputs, in-service battery life and performance history, external room environmental conditions, internal incubated environment chamber conditions, recent user behavior such as chamber opening/closing events, current state and/or history of power utilizations across the various subsystems within the incubated system 100, current state and/or history of the thermal control subsystem 132, current state and/or history of fluid exchanges via the fluid interface subsystem 127, optical subsystem 134 current state and/or history, current state and/or history of gas exchanges via the pneumatic interface subsystem 117, current state and/or history of any addition electrical interfaces 135 present in the chamber.

In some examples, the user interface subsystem 103 includes a display screen (such as but not limited to provide by an OLED array) and touch interface, providing the user the ability to observe limited, but key information about the incubated chamber 139 environment (such as, but not limited to, Temperature, Relative Humidity) on an ongoing basis. Using the touch interface, the user is also able to access a user configuration menu and interact with the incubator system 100 to address alarms, events and warnings provided by the controller subsystem 106. In some examples, additional status lights, knobs, buttons and switches are provided to allow the user to quickly review status and set important parameters. In yet another embodiment, the user interface subsystem 103 provides neither display screen nor touchscreen, with most interactions conducted by a device-linked software application, via the communication interface 107.

In some examples, the fluidic subsystem 109 includes a coupling interface to temporarily accept a reagent and waste cartridge 110. The reagent and waste cartridge is user-loaded and comprising of label and handling feature to aid user to load cartridge correctly. The reagent side of the cartridge is coupled to its respective reservoir and connected via a set of pump and valve to deliver reagents to the fluidic interface subsystem 127, and thereby into the culture dish 128. Similarly the waste side of the cartridge is coupled to its respective reservoir and connected via a set of pump and valve to extract reagents from the fluidic interface subsystem 127, and thereby from the culture dish 128. In some examples, multiple reagent and waste cartridges are accepted to deliver different reagents into the culture dish 128, via a plurality of fluidic pathways. In some embodiments, the reagent may be water to allow for active delivery of water droplets into the incubated chamber onto humidity stick 124 and/or onto an ultrasonic mister (not shown) added inside the chamber. In other embodiments, the reagent may be, but not limited to, a synthetic or biological drug, genetic manipulating agent, media, additional cells or staining chemicals. In some examples, the reagent and waste cartridges may be separated to compact the size by utilizing a single waste cartridge.

In some examples, the system sensor subsystem 108 includes (a) cartridge presence sensors used to detect the presence or absence of the reagent & waste cartridge 110 and the gas cartridge 114, (b) gas pressure and/or flow sensors used to detect the remaining capacity of the gas cartridge 114, (c) chamber state sensors used to detect the state of the chamber, such as but not limited to, incubated chamber open or close via the proximity sensor 141, (d) tilt sensors used to detect incubator system 100 orientations during placement on a surface or during transportation, and (e) external gas sensors, in the configuration when chamber gas is sampled and sensed outside the incubated chamber 139. In addition to these sensors, additional identification sensors (not shown) can be implemented to confirm the authenticity of the cartridges and to prevent the system from faulty use conditions. In some examples, a plurality of these sensors can be implemented to match the plurality of cartridges.

In some examples, the gas subsystem 112 includes (a) a coupling interface 113 (e.g., coupling port) receiving a user-loadable gas cartridge 114, and interfaced to a pressure regulator that has input pressure capacity of >1200 psi (and/or other gas tank pressures used for gas tank(s)). In some examples, the gas subsystem 112 can receive more than one gas cartridge 114, for instance receiving different gas cartridges 114 with different gases in them. In an illustrative example, the gas subsystem 112 can receive a first gas cartridge 114 with carbon dioxide (CO₂) and a second gas cartridge 114 with Nitrogen gas (N₂). The gas subsystem 112 includes (b) a pressure regulator provides a stepped down pressure supply (e.g., a range of 0-20 psi). The gas subsystem 112 includes (c) a valve interfaced to the pressure regulator that opens or closes for a brief duration of time (e.g., a range of 0-5 seconds) and interfaces with a constricted pneumatic line. The valve outputs the gas towards the downstream constrictor, however, also includes a path to vent the pressurized gas to the atmosphere via a port 115, if so chosen by the user. Such a need may arise if the user wishes to replace a cartridge prior to it being fully empty or if an incorrect cartridge was loaded. The gas subsystem 112 includes (d) a constricted pneumatic line implemented by, for instance, a miniature orifice or small ID tube, further controls the amount of gas supplied to the incubated chamber 139 via the pneumatic interface subsystem 117. Control of gas injections ensures gas percentages do not overshoot the set-point. An alternative embodiment can incorporate a sampler pump in the gas subsystem 112 that allows for sampling the gas environment of the incubated chamber 139 and flooding external (outside incubated chamber 139) gas sensors, while removing the need for maintaining gas sensors within the incubated chamber 139. An alternative embodiment includes a plurality of gas cartridges either as a backup for the primary cartridge and/or comprised of different gases. Such an embodiment will require multiple regulators, however, may utilize a valve with multiple selectable input pathways and two outputs (one that is directed towards the constrictor and the other to the vent port 115). In some examples, the incubator system 100 can include an ultra-fast valve (open and close times of <100 msec) that can eliminate the need for a downstream constrictor. In some examples, the gas subsystem 112 can include additional air purge pumps to purge the incubated chamber environment with atmospheric air and/or regulate the gas concentration in case of overshooting from set-point values. In some examples, the gas subsystem 112 can include controlled replenishment of atmospheric oxygen into the chamber, given that cell cultures consume oxygen which may get depleted in the environmental chamber 139.

In some examples the gas cartridge 114 includes a male thread and frangible seal, which couples to a female thread and piercing port within the coupling interface 113. The gas cartridge includes human-interface finger contoured features to aid twisting the cartridges into the coupling interface 113 and ensuring a secure seal. The gas cartridge also contains physical features that can be detected by the cartridge presence sensor on successful loading. In alternate embodiments, the gas cartridge also contains identification features (such as but not limited to optical barcodes, RFID, NFC tags) to further authenticate the cartridge and with accompanying sensors in the system sensor subsystem 108. In an alternative embodiment, the gas cartridge 114 and coupling interface 113 may interface via quick-disconnecting using spring-based sealing and opening mechanisms that are commonly used in pressurized air hose plug-and-socket adapters.

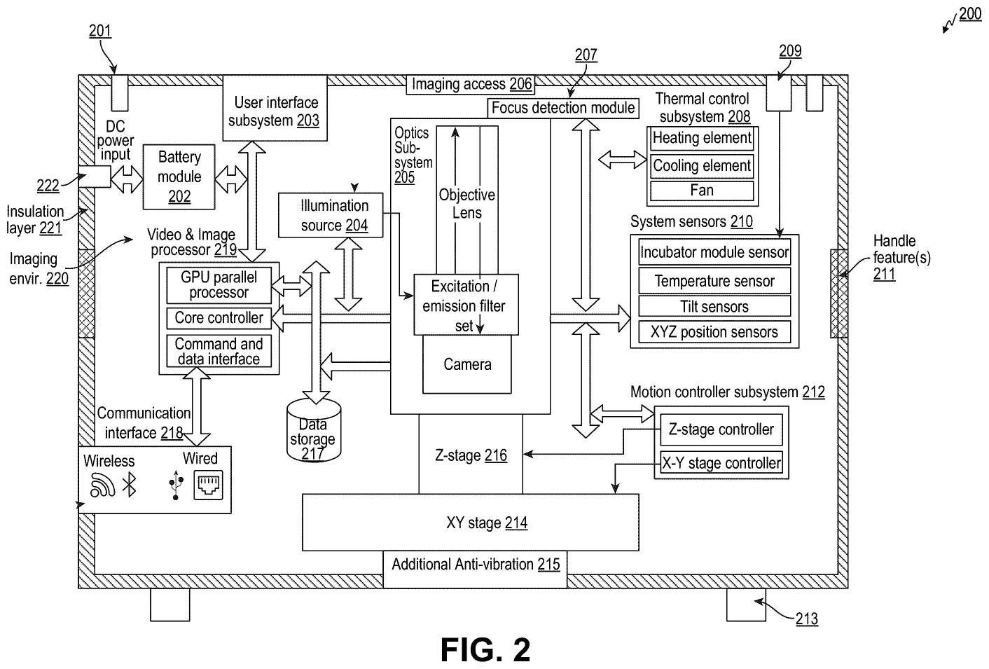

FIG. 2 is a cutaway architecture diagram illustrating an imaging system 200. In some examples, the imaging system 200 can be referred to as an imaging subsystem, for instance when the imaging system 200 is used as a subsystem of a combined incubation and imaging system (e.g., incubation and imaging system 300 of FIG. 3).

Various subsystems, components, and interfaces of the imaging system 200 are illustrated in FIG. 2. In some examples, the imaging system 200 includes, or encompasses, an imaging environment 220, which is insulated from the external world by an insulation layer 221. The insulation layer 221 (which can be made from, for instance, fiber glass, foam, and/or ceramic composites) reduces thermal exchanges from the external world into or out of the imaging environment, to better control the temperature of the imaging system 200 and/or the incubator system 100 from fluctuations. In some examples, such an insulation feature may not be a separate layer, however, the functionality offered by the chassis material (such as but not limited to bent sheet metal, injection molded plastics, 3D printed materials and ceramics) of the imaging system 200.

The imaging system 200 includes an alignment feature 201 that is used to align with an incubator system 100, which ensures that both subsystems are spatially oriented as needed for the functionality of the integrated unit. The imaging subsystem includes imaging access port 206 (e.g., microscopy access port) which allowing the optics subsystem 205 access to the culture dish 128 in the incubator system 100. In some embodiments, the imaging access port 206 may already be open and not requiring removal and in other embodiments, it can be mechanically linked to self-open and close when integrating with an incubator subsystem. Additionally, a proximity sensor 209 is provided on the imaging subsystem to allow for detection of the physical integration. In some examples, such a proximity sensor (such as, but not limited to using a reed-switch and adjoining magnet) may be housed in the incubator subsystem, to accomplish the same functionality. The imaging subsystem includes handle features 211 that allow the users to transport the subsystem easily and includes anti-vibration dampening legs 213 to reduce bench noise from entering the imaging environment. In some examples, the handle features 211 may be designed within the outer chassis of the imaging subsystem. In some examples, the handle features 211 may be accomplished using belts and/or externally added handles.

In some examples, the DC power input 222 allows for plugging into an external supply to power the imaging system 200 and charge the battery subsystem 202. When the DC power input is unplugged, the imaging system 200 continues to be powered by the battery subsystem 202. The battery subsystem can be made of rechargeable material, such as but not limited to, Lithium-ion, Lithium-poly, Nickel-Metal Hydride or Nickel-Cadmium. In some examples, the unit may be powered by a single-use battery. In some examples, the battery subsystem 202 powers the video & image processor 219, which then distributes power to the remaining subsystems in the imaging system 200. In some examples, the battery subsystem may be connected to a power-distribution subsystem (not illustrated in FIG. 2), which in turn distributes the power.

In some examples, the video & image processor 219 includes a micro-processor or micro-controller board that is pre-loaded with a firmware program, with all computing occurring on a single processing core. In some examples, the computing occurs on a micro-processor or micro-controller board with additional parallel graphic processing unit (GPU) co-processors. The firmware program running on the subsystem, comprises a command and data interface routine for communicating with the external world via the communication interface 218. This routine accepts commands, such as but not limited to, set-points, calibration curves, illumination control, image and video acquisition and configuration parameters to allow the user to control the imaging system 200. The command and data interface routine also transmits sensor data, video & image data (raw or processed), events, warnings and logs to the user for monitoring the performance, functionality, and reliability of the imaging system 200. The communication interface 218 provides a wireless (via protocols such as but not limited to WiFi, Bluetooth, Cellular) and/or wired (via protocols such as but not limited to USB, Ethernet, serial port, I2C, MOSI).

In some examples, the video & image processor 219 includes a core controller routine, which accepts the control information provided by the user, and along with feedback from the system sensor subsystem 210 data, controls the various subsystems and components in the imaging environment 220, including but not limited to the thermal control subsystem 208, the optics subsystem 205, the Illumination source 204, Data storage 217, motion controller subsystem 212, Focus detection subsystem 207 and user interface subsystem 203. The core controller routine includes an alarm and event coordinator sub-routine, to read sensor information from the system sensor subsystem 210 and warn the user of the state of the imaging subsystem. In some examples, the core controller routine may also include additional sub-routines, such as long-term adaptive machine learning for better control accuracy and/or predictions of system performance. For instance, a machine learning algorithm and/or model can monitor the temperature of the incubated chamber 139 and change heating parameters in order to reduce the time it takes to arrive stably within a specified set-point. Inputs into the learning algorithm and/or model can include external environment temperature, integration event history with the incubator system 100, heater component and fan life, current state and/or history of the optical subsystem 205, current state and/or history of the illumination source 204, current state and/or history of the motion controller subsystem 212, current state and/or history of the video & image processor 219. In some examples, a machine learning algorithm and/or model can predict battery subsystem 202 charge capacities, the implementation of which would further aid the user to assess and/or plan available culture imaging time without DC power input 104. In this instance, the machine learning algorithm and/or model may utilize, as inputs, in-service battery life and performance history, external room environmental conditions, image(s) and/or video(s) captured by the optical subsystem 205, image capture settings (e.g., focus, exposure time, aperture size, zoom, white balance, analog gain, digital gain, level of illumination) used by the optical subsystem 205 to capture image(s) and/or video(s), image processing settings (e.g., brightness, contrast, color saturation, tone mapping, noise reduction, sharpening) used by the video & image processor 219 to process image(s) and/or video(s) captured using the optical subsystem 205, internal imaging environment chamber system sensor subsystem 210 conditions, recent user behavior such as incubator system 100 integration events, current state and/or history of power utilizations across the various subsystems within the imaging system 200, current state and/or history of the thermal control subsystem 208, current state and/or history of the motion controller subsystem 212, current state and/or history of the optical subsystem 205, current state and/or history of the illumination source 204, current state and/or history of usage of the data storage 217, current state and/or history of the thermal control subsystem 132 in the integrated incubator system 100, current state and/or history of the system sensor subsystem 108 in the integrated incubator system 100. In some examples, the machine learning algorithm and/or model can enhance the ability and speed to achieve and/or maintain focus, inputs to which may include video and/or stacks of image frame data acquired and processed by the video & image processor 219, current state and/or history of the focus detection subsystem 207, current state and/or history of the sensors of the system sensor subsystem 210 (including in addition to described in FIG. 2, a vibration sensor), current state and/or history of the thermal control subsystem 208, current state and/or history of the motion controller subsystem 212, additional user provided information such as culture dish 128 type, size and/or count. Outputs of such a machine learning algorithm and/or model can modulate the current state and/or future state of the thermal control subsystem 208, current state and/or future state of the motion controller subsystem 212, current state and/or future state of the focus detection subsystem 207, current state and/or future state of the video & image processor 219, and/or current state and/or future state of the optics subsystem 205. In some examples, a machine learning algorithm and/or model can detect and/or predict occurrence of a specified condition(s) and/or event(s) associated with the culture, such as at least a specified amount of a particular set of one or more cell(s) or tissue(s) dying, a particular set of one or more cell(s) or tissue(s) growing beyond a threshold amount, at least a specified amount of a particular set of one or more cell(s) or tissue(s) undergoing a specified modification, fluorescence of a specified amount of a particular set of one or more cell(s) or tissue(s) in a specific color or light frequency range (e.g., based on a biomarker), or a combination thereof. In some examples, the machine learning algorithm can apply computer vision techniques to image(s) and/or video(s) captured using the optical subsystem 205, such as feature extraction, feature detection, feature recognition, feature tracking, object detection, object recognition, object tracking, image classification, object classification, semantic segmentation, or combinations thereof. In some examples, a machine learning algorithm and/or model can determine image capture setting(s) (e.g., focus, exposure time, aperture size, zoom, white balance, analog gain, digital gain, level of illumination) for the optical subsystem 205 to use for further capture of image(s) and/or video(s) based on previous image capture setting(s), previous image processing setting(s), previous image(s) and/or video(s), predicted changes to the culture (e.g., predicted increased brightness due to bioluminescent biomarkers), or combinations thereof. In some examples, a machine learning algorithm and/or model can determine image processing setting(s) (e.g., brightness, contrast, color saturation, tone mapping, noise reduction, sharpening) used by the video & image processor 219 to process image(s) and/or video(s) captured using the optical subsystem 205 based on previous image capture setting(s), previous image processing setting(s), previous image(s) and/or video(s), predicted changes to the culture (e.g., predicted increased brightness due to bioluminescent biomarkers), or combinations thereof.

In some examples, the user interface subsystem 203 includes a display screen (such as but not limited to provide by an OLED array) and touch interface, providing the user the ability to observe limited, but key information about the imaging environment (such as, but not limited to Temperature, Video and Image data) on an ongoing basis. Using the touch interface, the user is also able to access a user configuration menu and interact with the imaging system 200 to address alarms, events and warnings provided by the video & image processor 219. In some examples, additional status lights, knobs, buttons and switches are provided to allow the user to quickly review status and set important parameters. In yet another embodiment, the user interface subsystem 203 provides neither display screen nor touchscreen, with most interactions conducted by a device-linked software application via the communication interface 218.

In some examples the motion controller subsystem 212 is housed on top of an additional anti-vibration component, to further reduce external and internal vibrational disturbances while the optics subsystem 205 (installed on the motion controller subsystem 212) is performing an imaging operation. The motion controller subsystem 212 controls the position of an XY stage and Z-stage to enable positioning the optical subsystem at various spatial locations to perform microscopy on the culture dish 128. The XY and Z stages can be made using an assembly of linear actuators using various mechanical mechanisms, including but not limited to being belt-driven, lead screw-driven, voice-coil or piezo-based actuation. The Z-stage is coupled with the optical subsystem 205 and aids in focusing of lenses for imaging purposes. In an alternative embodiment, a plurality of XY and Z stages are used with one or a plurality of motion control subsystems, to enable the use of a plurality of optical subsystems 205. Such an embodiment would allow for simultaneous imaging of multiple culture dish locations and/or allow for reducing the spatial travel required of the stages. In an alternative embodiment, components of the optical subsystem can be placed on an additional stage (such as but not limited to a rotating stage), to present various objective lens and thereby microscopy magnifications for imaging purposes, which is also controlled by the motion controller subsystem 212.

In some examples, the optics subsystem 205 includes a microscopic objective lens, and excitation & emission filter set and an imaging camera, and is coupled to an Illumination source 204. The illumination source 204 provides light at known wavelengths (utilizing sources such as but not limited to light-emitting diodes LEDs, lasers or filament-based), to enable illuminating the samples in the culture dish 128, via an inverted microscopy mode (whereby the excitation light travels through the objective lens to the sample and the emitted or reflected light follows a similar path back to the camera). In an alternative embodiment, the illumination source 204 can be included within the optics subsystem 205 for further compactness. In some examples, the excitation and emission filter set allows for epi-fluorescent microscopy, and designed to match the illumination source and fluorescence wavelengths of the samples in the culture dish 128. In an alternative embodiment, the excitation and emission filter set may not be present if the application does not require filtering of specified wavelengths, such as but not limited to bright-field microscopy, dark-field microscopy, differential interference contrast or phase-contrast microscopy. In an alternative embodiment, the illumination source 204 may not be present, when utilizing the optical subsystem 134 light source present in the incubator system 100, in the case of performing trans-illumination microscopy.

In some examples, image or video data acquired by the camera in the optic subsystem 205 are transmitted to the video & image processor 219. Such data transmission can utilize communication protocols such as, but not limited to, Camera-link, CoaXpress, USB, Camera Serial Interface or Ethernet. The image or video data can be transmitted in raw uncompressed format or compressed format, depending on the user settings and camera capture needs. The video & image processor 219 allows for real-time frame-by-frame computing and analysis of the incoming data. Computing and analysis algorithms can include, but not limited to, image processing pipelines starting with raw image adjustments, feature segmentation, identification, counting, tracking and quantitation. In some examples, GPU parallel processing routings in the video & image processor 219 can implement machine learning algorithms to train a predictive model. In an alternative embodiment, the image or video data may also be transmitted to a data storage 217 (using but not limited to storage technologies such flash memory, solid-state drive or disc-drives), that can be later retrieved by the video & image processor 219. For instance, cellular and/or tissue related features extracted by the machine-learning algorithm on processing of video and/or image frames, can be used to predict outcomes in various use cases such as but not limited to healthy vs. disease states for purposes of diagnosis, phenotype of interest vs. non-phenotype of interest for biological research, contamination vs. not contaminated for quality control, drug-sensitive vs. not drug-sensitive for drug discovery, infectious vs non-infectious for virology. In such instances, the machine-learning model itself can be trained on culture sets encompassing sub-sets of both and/or multiple known outcomes. In some instances, alternative artificial intelligence methods such as Deep Learning may be applied directly to large known outcome culture sets and/or to longitudinal imaging sets on a single culture (in which case the outcome at various time-points is known and/or controlled by the user per the application), to inherently train the learning model using video data. Once trained, the model may then be used to predict culture outcomes on unknown culture sets and/or longitudinal time-points. In some examples, the learning models using the ML and/or Deep Learning approaches may be generated ‘on-board’ the integrated system. In other examples, the raw and/or processed videos and images may be uploaded ‘off-board’ the integrated system to take advantage of high-performance computing environments offered in the cloud. The learning models generated ‘off-board’, may then be downloaded ‘on-board’ once the training is partially and/or fully completed.

In some examples, the thermal control subsystem 208 is placed within the imaging environment 220 includes a heating element, cooling element and a circulating effector such as a convective fan or blower. In some examples a cooling element may not be necessary to arrive at temperature set-points and only a heating element is provided. Heating and cooling elements may be implemented using a wide variety of available electronic components, including but not limited to, peltier-based thermoelectric heater-cooling, resistive heaters and ceramic heating. There may be one or several heating elements, cooling elements and/or convective elements placed inside the imaging chamber to accomplish uniform heat distribution. In some examples, the thermal control subsystem 208, the core-controller routing in the video & image processor 219 and a temperature sensor (or a plurality of sensors) in the system sensor subsystem 210, include components in a feed-back system, used to maintain the temperature of the optics subsystem 205 within set tolerances (+/−5 degrees Celsius or less) of the temperature measured in the incubated chamber 139. The utility of doing so is to prevent focus drifts caused due to temperature differences at the objective lens in the optics subsystem 205, at the microscope access port 206 interface.

In some examples, camera focusing is achieved by acquiring a series of images “image stack” by moving the z-stage in step increments and then analyzing the images in the video & image processor 219, to arrive at the z-stage position for best focus. Auto-focusing algorithms can implement, but not limited to, contrast-based, phase-based or image file size-based methods, either of which can be user selected. In an alternative embodiment, a focus detection subsystem 207 can be included, comprising of an optical method to locate the z-height of the surface that the sample resides on in the culture dish 128. Such a focus detection subsystem 207 can include a compact off-axis laser light coupled into the objective lens in the optic subsystem 205. Given the off-axis nature of the laser light, the light is reflected from the sample surface is captured at different XY locations across the camera sensor plane, which can then be calibrated to ascertain focus z-height. In some examples, the focus detection subsystem 207 can directly direct a tiny light spot at the sample surface and capture reflected light onto an internal camera subsystem, utilizing geometric changes and/or time-of-flight methods to determine focus offsets.

In some examples, the system sensor subsystem 210 includes (A) a sensor used to detect the state of the chamber, such as but not limited to, incubator subsystem presence or absence via a proximity sensor (e.g., proximity sensor 209), (B) a sensor used to detect the temperature at the optic subsystem 205, (C) tilt sensors used to detect imaging system 200 orientations during placement on a surface or during transportation, and/or (D) XYZ position sensors, to monitor spatial position of the XY and Z stages.

FIG. 3 is a cutaway architecture diagram illustrating the incubator system 100 and the imaging system 200 combined to form an incubation and imaging system 300. Several aspects of an ‘integration event’ ensure that both the subsystems are spatial, electrically and optically coupled with each other. To ensure desired spatial orientation of both subsystems, FIG. 3, depicts a scheme where the alignment features 137 on the incubator system 100 mate with the alignment features 201 of the imaging system 200. Retaining the alignment can occur simply due to the forces of gravity. In some examples, additional magnets and mechanical latches can be implemented to further constrain the spatial mating of the subsystems. To ensure optical coupling of the subsystems, the imaging access port 129 of the incubator system 100 and the microscopy access port 206 of the imaging system 200 can be manually removed by the user and/or automatically moved via mechanical linkages at the successful completion of the alignment. A proximity sensor 209 on the imaging subsystem confirms successful physical integration of the two subsystems. For electrical coupling the communication interface 107 of the incubator system 100 can be connected to the communication interface 218 of the imaging system 200, using external wires such as, but not limited, to a USB cable, a Ethernet cable, a Serial interface cable, ribbon cables with I2C or MOSI channels. In an alternative embodiment, an internal electrical connection can be made from the top module 101, to the bottom module 102 of the incubator subsystem and finally into the imaging system 200 using an additional electrical interface port, implemented using a series of spring-loaded pogo-pin connectors. In an alternative embodiment, the incubator system 100 and imaging system 200, communicate information using wireless protocols (e.g., cellular networks, WLAN, WiFi and/or Bluetooth) and/or cloud-based network protocols.

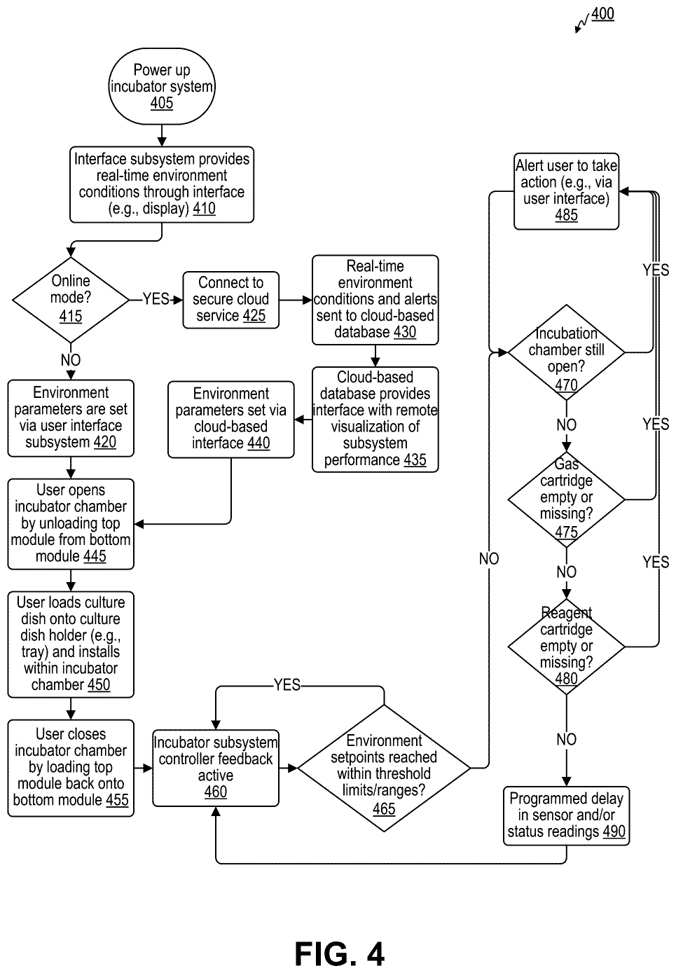

FIG. 4 is a flow diagram illustrating an exemplary process 400 for performing long-term cell or tissue culture incubation using the incubation system. The process 400 may include workflow operations for users operating the incubator system 100 and/or the incubation and imaging system 300, as well as system operations of the incubator system 100 and/or the incubation and imaging system 300.

The process 400, at operation 405, involves the user powering up the incubator system 100, then at operation 410 and/or operation 415, selecting (e.g., via user interface subsystem 103) whether the incubator subsystem performs in online mode or offline mode. When operating in online mode, at operation 425, the communication interface 107 establishes a connection (wired or wireless) to a dedicated secure cloud service (e.g., cloud-based database). At operation 430, the communication interface 107 sends conditions and additional alerts (associated with the incubated chamber 139) received from the controller subsystem 106 to the cloud service (e.g., the cloud-based database). At operation 435, the cloud-based service (e.g., cloud-base database) provides the user (e.g., through a cloud user interface or through user interface subsystem 103) with the ability to visualize subsystem performance and/or, at operation 440 control (e.g., set and/or modify) environmental parameters (e.g., remotely and/or locally). The database also stores long-term data and is analyzed (e.g., by ML model(s) 2225) for hardware reliability improvements. These can include, but not limited to, assessing control loop performance and predicting component failures in case conditions do not meet quality criteria.

If, at operation 415, the incubation and imaging system 300 is operating in offline mode (e.g., user selects offline mode), the environmental parameters are set via the user interface subsystem 103, itself (e.g., based on inputs received by the user interface subsystem 103).

At operation 445, whether the incubation and imaging system 300 is in the online mode or offline mode, the user then accesses the incubated chamber 139 and loads a culture dish 128 housed within a culture dish holder 125. At operation 450, the user closes the incubator chamber by loading the top module 101 back onto the bottom module 102.

At operation 460, incubator subsystem controller feedback is activated to help the controller subsystem 106 arrives at the environmental set points within acceptable limits (e.g., within threshold limits and/or ranges). If, at operation 465, the environmental set points are within the acceptable limits (e.g., within threshold limits and/or ranges), then the feedback can continue back to operation 460, optionally with a delay before checking again (at operation 465), to ensure that the environmental set points are maintained within the acceptable limits (e.g., within threshold limits and/or ranges) over time.

If, at operation 465, the environmental set points are not within the acceptable limits (e.g., not within threshold limits and/or ranges), then the incubator system 100 and/or the incubation and imaging system 300 can perform various checks, and alert the user if needed. At operation 470, if the system detects (e.g., based on measurements from sensors such as the proximity sensor 141) that the incubation chamber is still open, the system can go to operation 485 and alert the user (e.g., via the user interface subsystem 103) to take action to close the incubation chamber. At operation 475, if the system detects (e.g., based on measurements from sensors, such as the cartridge presence sensors) that a gas cartridge 114 is empty or missing, the system can go to operation 485 and alert the user (e.g., via the user interface subsystem 103) to take action to refill or replace the gas cartridge 114. At operation 480, if the system detects (e.g., based on measurements from sensors, such as the cartridge presence sensors) that a reagent cartridge (e.g., reagent & waste cartridge 110) is empty or missing, the system can go to operation 485 and alert the user (e.g., via the user interface subsystem 103) to take action to refill or replace the reagent cartridge.

For instance, in some examples, if the system sensor subsystem 108 reports the chamber open, gas cartridge missing or a reagent cartridge missing, it will enter an alert loop, until the situation is remedied by the user. In some examples, the user may connect additional electrical interfaces 135 to the culture dish 128 prior to loading it into the incubator subsystem and reversibly disconnect it when unloading. Data from the additional electrical interface 135 can also communicated to the cloud-database. In some examples, perfusion fluidic lines may be coupled between the fluidic interface subsystem 127 to the culture dish 128, during the loading steps and de-coupled during the unload steps.

If none of the checks (operations 470 through 480) identify an issue, then at operation 490, the incubator system 100 and/or incubation and imaging system 300 delays before collecting further sensor readings and/or status readings, and before returning to operation 460.

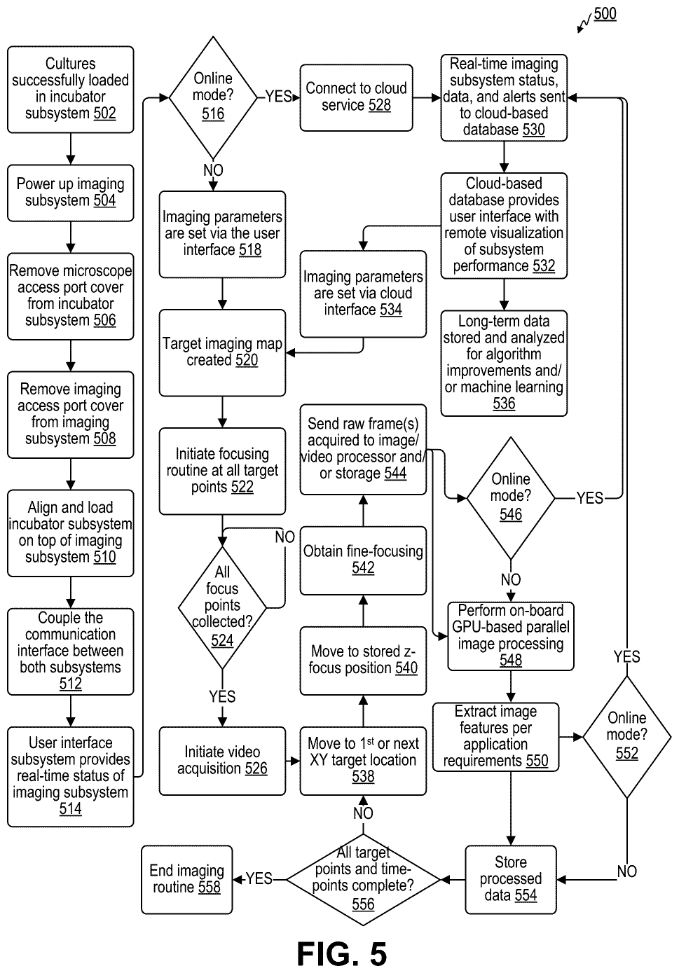

FIG. 5 is a flow diagram illustrating an exemplary process 500 for performing long-term cell or tissue culture imaging using the incubation and imaging system 300. The process 500 may include workflow operations for users operating the imaging system 200 and/or the incubation and imaging system 300, as well as system operations of the imaging system 200 and/or the incubation and imaging system 300.

In some examples, the process 500 includes the operations of the process 400, for instance to load culture dishes 128 into the incubator system 100, such as any of operations 405 through 455, or combinations thereof. At operation 502, the cultures are successfully loaded into the incubator system 100 (e.g., or incubator subsystem of the incubation and imaging system 300).

When an imaging session is desired, at operation 504, the user (and/or the system itself) power ups the imaging system 200. At operations 506 and 508, the user (and/or the system itself) removes the cover(s) from the imaging access port 129 of the incubator subsystem (e.g., incubator system 100) and/or from the imaging access port 206 of the imaging subsystem (e.g., imaging system 200), allowing for optical access between the subsystems of the incubation and imaging system 300. At operation 510, the two subsystems are loaded together and/or spatially aligned using the alignment features 137 and the alignment features 201 present on the subsystems. At operation 512, the user (and/or the system itself) couples (e.g., connects) the communication interface 107 and the communication interface 218 between the subsystems (e.g., using wired cables or wireless interfaces). In some examples, the communication interfaces connect as a result of aligning the two subsystems and bringing them together (via the use of, but not limited to, pogo-pin like electrical connectors) and the user does not have to perform this extra step manually. In some examples, at operation 514, the user interface subsystem 103 provides real-time updates as to the status of the incubator subsystem, imaging subsystem, or both.

At operation 516, the user (and/or the system) selects whether the imaging subsystem performs in online mode or offline mode via the user interface subsystem 203.

When operating in online mode, at operation 528, the communication interface 218 establishes connection (wired or wireless) to a dedicated cloud service (with a cloud-based database), where, at operation 530, the communication interface 218 sends conditions of the imaging environment 220, sensor status, imaging data, and/or additional alerts received from the video & image processor 219. At operation 532, the cloud-based database provides the user with the ability to remotely visualize subsystem performance, and, at operation 534, control (e.g., set and/or modify) imaging parameters via an interface (e.g., cloud interface, the user interface subsystem 103, and/or user interface subsystem 203). At operation 536, the database can also store long-term data that is analyzed for hardware reliability improvements, image algorithm improvements and machine learning. These can include, but not limited to, assessing performance of the imaging system 200 and predicting component failures in case conditions do not meet quality criteria.

If, at operation 516, the system is in offline mode (e.g., as selected by the user and/or the system), then at operation 518, the imaging parameters are set via the user interface subsystem 203 (and/or the user interface subsystem 103).

At operation 520, whether the system is in online mode or offline mode, the user and/or system creates a target imaging map where imaging is expected to occur. At operation 522, the system initiates a focusing routine at all target points, for instance performing auto-focusing procedures, for instance looping through (at operation 524) until data for all focus points is collected. At operation 526, the system initiates video acquisition.

At operation 538 and operation 540, the motion controller subsystem 212 commanding the XY stage 214 and Z-stage 216 to the various target map positions (e.g., for instance moving to a first or next XY target location at operation 538, and/or moving to a stored z-focus position at operation 540), and performing auto-focusing procedures. Once an initial focus map is obtained, the imaging subsystem loops through to the target map positions performing additional fine-focusing (at operation 542) and acquiring raw video or image frames by the optics subsystem 205. At operation 544, the raw data can be sent to the video & image processor 219 for real-time image processing (e.g., GPU-based) (at operation 548) and/or sent to storage 217 (at operation 544 and/or operation 554). The video & image processor 219 can extract image features according to the needs of the particular application (e.g., experiment) being run on the incubation and imaging system 300 (at operation 550). At operation 554, the extracted image features can be further sent to storage for later retrieval. If, at operation 556, all target points and time points are complete, then at operation 558, the process 500 is complete, and ends. If at operation 556, all target points and time points are not complete, the process 500 can return to operation 538.

In the case of online mode configurations (e.g., via operation 546 and/or operation 552), then raw data frames and/or extracted image features can be sent to the cloud-database for real-time remote access (e.g., at operation 530). In some examples, the user may have additional steps to change out or replace optical subsystem add-ons (like excitation/emission filters, sources) prior to initiating the imaging routine (e.g., the process 500). In some examples, these steps are conducted for long durations of times exceeding a threshold amount of time (e.g., for one or more minute(s), hour(s), day(s), week(s), month(s), and/or year(s)), all the while the system recording raw videos and/or processed videos of the cultures that are maintained in the incubated chamber 139. The system may provide the user physical alerts and/or notifications via electronic messaging if any of the imaging subsystem's sensors in the system sensor subsystem 210, system sensors in the system sensor subsystem 108 and/or an adaptive machine learning algorithm in either the video & image processor 219 or control subsystem 106 or both, find incubation and/or imaging environment parameters outside of previously set bounds and/or if the system is detected to be in an error state, or detect a specific condition or event in the culture (e.g., an amount of cells or tissues has died, grown by at least a growth amount, multiplied by at least a multiplication amount, fluoresced in a color and/or frequency range associated with a biomarker, and/or been modified in a specified way), thereby allowing the user to take appropriate action (e.g., correct the error state, advance the experiment to a next stage, and the like). In some examples, processing of the video and/or image data itself may lead to these alerts and notifications, such as for instance when the imaging subsystem cannot find focus for more than a set amount of time, frame brightness-levels or contrast or background noise are outside acceptable thresholds, frame drift is detected signifying unacceptable movement of culture plate or imaging XYZ stages, non-uniform illumination and/or de-focus across the field of camera view. In some examples, the alerts and/or notifications are caused by processing of culture and tissue related parameters, such as for instance cellular morphology characteristics, cellular growth characteristics, cellular movement dynamics, internal sub-cellular features, external cellular derived features, molecular properties within the cellular environment, inter-cellular features, cellular organization within a tissue, tissue organization within an organoid or organ, internal cellular and/or tissue biochemical parameters and/or biochemical parameters in the culture dish, presence of unwanted microbes and/or microorganisms indicative of possible contamination, physio-chemical properties of the culture media. This system of notifications and alerts can improve flexibility of the system and efficiency of a laboratory as a whole, allowing the system to continue incubating and monitoring a culture without user input or intervention, allowing a user to perform other tasks with the knowledge that the system with notify or alert the user if and when user input or intervention is needed.

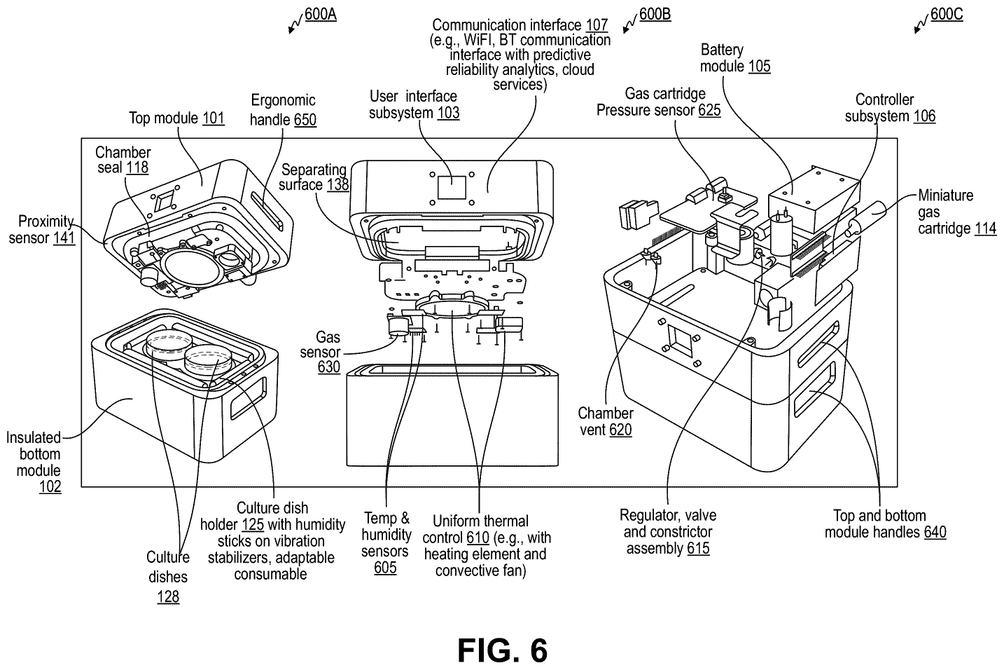

FIG. 6 includes exploded perspective views 600A-600C of the incubation and imaging system 300. In a first exploded perspective view 600A, two culture dishes 128 are placed into the incubated chamber 139 in the bottom module 102 (which is insulated), on a culture dish holder 125 with a culture dish 128 with humidity sticks on vibration stabilizers. The top module 101 is also visible, with an ergonomic handle 650 (e.g., an example of the handle features 116), chamber seal 118, and proximity sensor 141 all visible.

In a second exploded perspective view 600B, the top module 101 is illustrated with the user interface subsystem 103, the communication interface 107, and the separation surface 138 visible and marked. The communication interface 107 can include WiFI and/or Bluetooth communication interface(s) with predictive reliability analytics and/or cloud services. Several components are illustrated between the top module 101 and the bottom module 102. These components can be part of the top module 101, the bottom module 102, or both. These components can include a temperature and humidity sensors 605, uniform thermal control 610 (e.g., with a heating element and/or a convective fan), a gas sensor 630, and/or other components. In some examples, the temperature and humidity sensors 605 may be part of the chamber sensor subsystem 133. In some examples, the uniform thermal control 610 can be part of the thermal control subsystem 132. In some examples, the gas sensor 630 can be part of the system sensor subsystem 108 and/or the chamber sensor subsystem 133.

In a third exploded perspective view 600C, the top module 101 is illustrated in an exploded fashion, with several components being visible. These components include the battery module 105, the controller subsystem 106, the gas cartridge 114, a regulator valve and constrictor assembly 615 (e.g., which may be, or may be part of, the gas subsystem 112), a chamber vent 620 (e.g., gas subsystem 112, pneumatic interface subsystem 117, check valve 130, relief valve 142), a gas cartridge pressure sensor 625 (e.g., part of the system sensor subsystem 108 and/or the gas subsystem 112 that sensors pressure of the gas cartridge 114), and top and bottom module handles 640 (e.g., handle features 116, handle features 119).

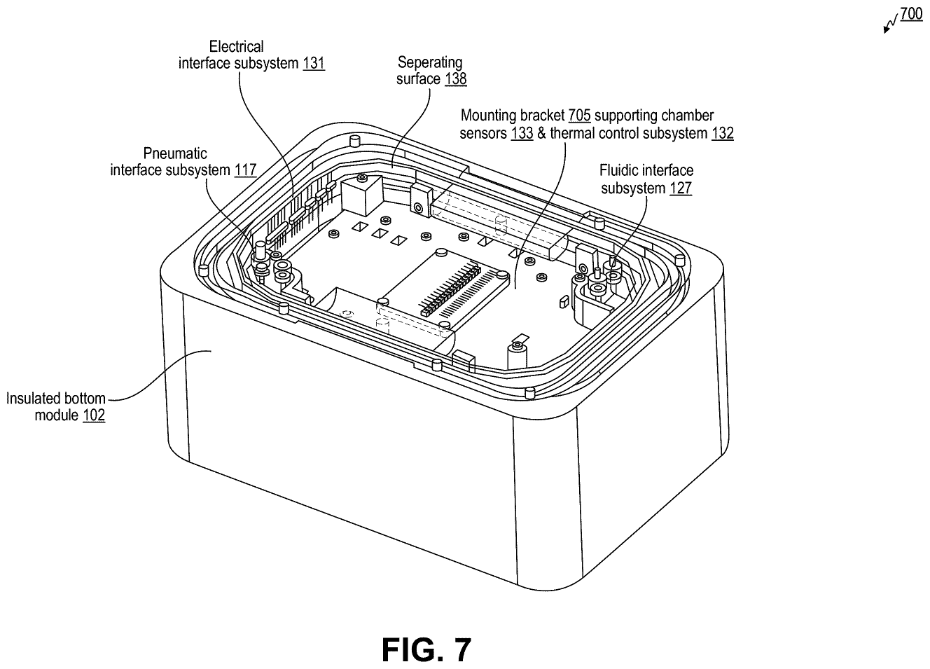

FIG. 7 is a perspective view 700 of the bottom module 102 (e.g., insulated) of the incubator system 100. Components visible through the top of the bottom module 102 in the perspective view 700 include the pneumatic interface subsystem 117, the electrical interface subsystem 131, the separation surface 138, the fluidic interface subsystem 127, and a mounting bracket 705 supporting the thermal control subsystem 132 and the sensors of the chamber sensor subsystem 133.

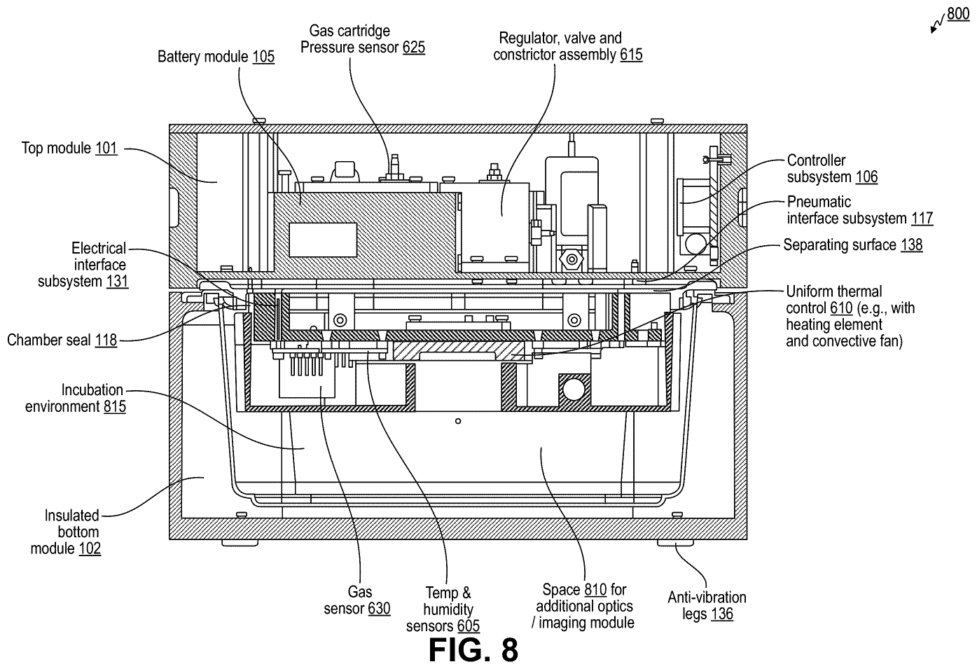

FIG. 8 is a cutaway architecture diagram 800 illustrating the incubation and imaging system 300. Some of the components of the incubation and imaging system 300 that are visible in the cutaway architecture diagram 800 include the top module 101, the bottom module 102, the battery module 105, the controller subsystem 106, the pneumatic interface subsystem 117, the chamber seal 118, the electrical interface subsystem 131, the anti-vibration legs 136, the separation surface 138, the temperature and humidity sensors 605, the uniform thermal control 610, the constrictor assembly 615, the gas cartridge pressure sensor 625, the gas sensor 630, a space 810 for additional optics and/or imaging subsystems (e.g., imaging system 200), and an incubation environment 815 (e.g., incubated chamber 139).

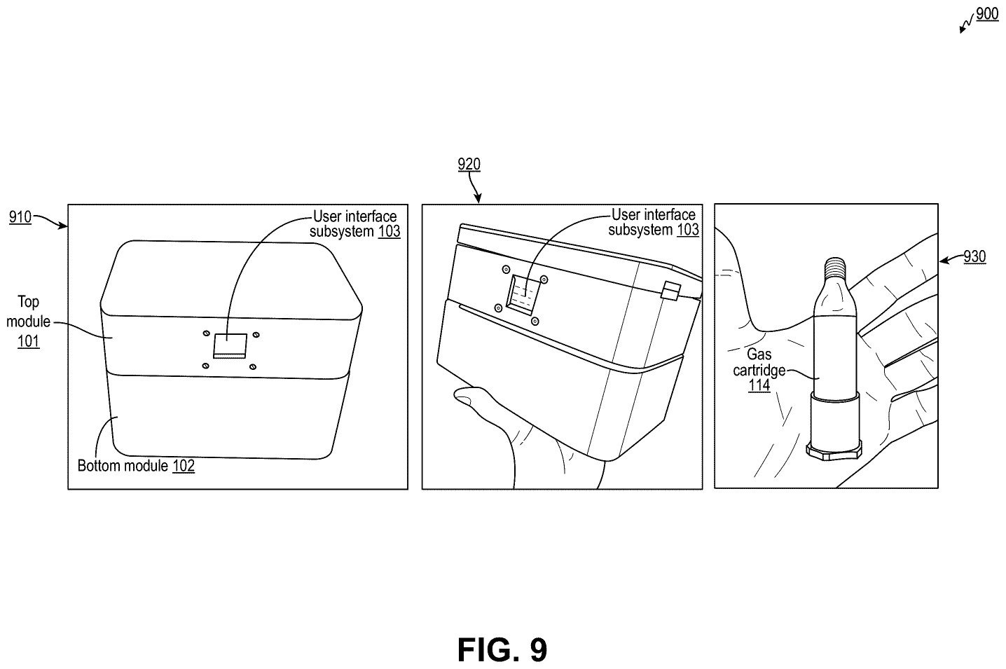

FIG. 9 includes a front view 910 of the incubation and imaging system 300, a perspective view 920 of the incubation and imaging system 300, and a side view diagram 930 of a gas cartridge 114. In the front view 910 and the perspective view 920, certain components of the incubation and imaging system 300 are visible, including the top module 101, the bottom module 102, and the user interface subsystem 103. In the side view diagram 930, the gas cartridge 114 is illustrated in the palm of a user's hand, showing how small the gas cartridge 114 can be. In the perspective view 920, the incubation and imaging system 300 is illustrated held in a user's hand, also showing how small the incubation and imaging system 300 can be. In the view 910, the incubation and imaging system 300 is powered off and at rest. In the view 920, the incubation and imaging system 300 is powered on and held single-handedly, showcasing the portability of the incubation and imaging system 300.

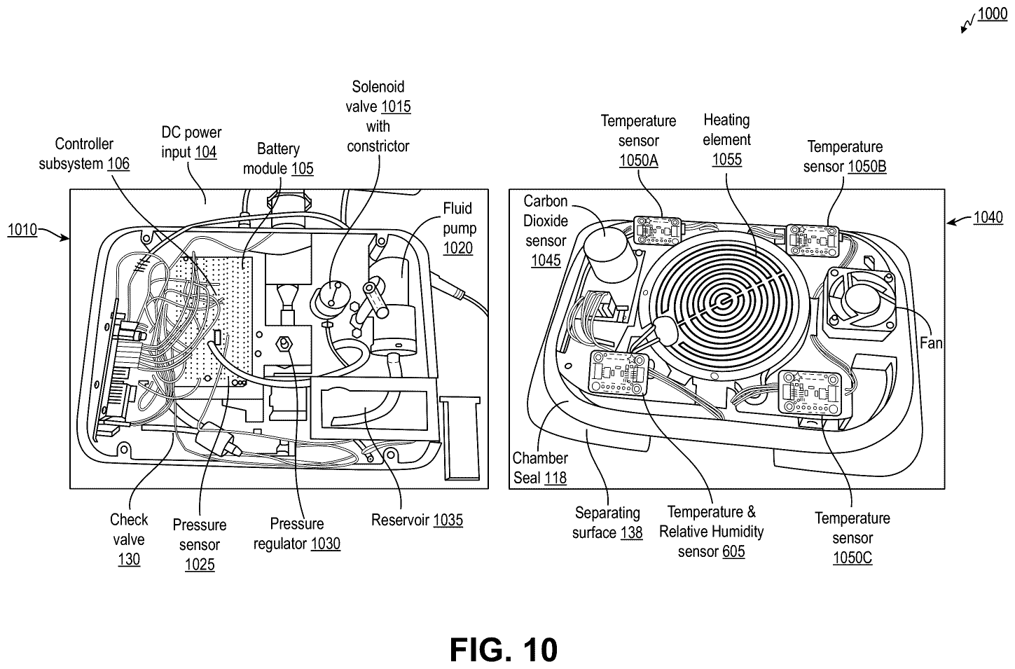

FIG. 10 includes internal views (view 1010, view 1040) of internal components of the incubation and imaging system 300. In view 1010, several components of the incubation and imaging system 300 are visible, including the DC power input 104, the battery module 105, the controller subsystem 106, the check valve 130, a solenoid valve 1015 with a constrictor (e.g., which may be the valve and constrictor of the gas subsystem 112), a fluid pump 1020 (e.g., of the fluidic subsystem 109), a pressure sensor 1025 (e.g., of the system sensor subsystem 108), a pressure regulator 1030 (e.g., of the regulators of the gas subsystem 112), and a reservoir 1035 (e.g., of the fluidic subsystem 109). In some examples, the components visible in the view 1010 may be in the top module 101.

In view 1040, several components of the incubation and imaging system 300 are visible, including the chamber seal 118, the separation surface 138, the temperature and humidity sensors 605, a carbon dioxide sensor 1045 (e.g., of the chamber sensor subsystem 133), three temperature sensors 1050A-1050C (e.g., of the chamber sensor subsystem 133 and/or system sensor subsystem 210), and a heating element 1055 (e.g., of the thermal control subsystem 132 and/or thermal control subsystem 208). The view 1040 may represent an example layout of the chamber sensor subsystem 133, the incubated chamber 139, the thermal control subsystem 132, the chamber seal 118, and/or separating surface 138.

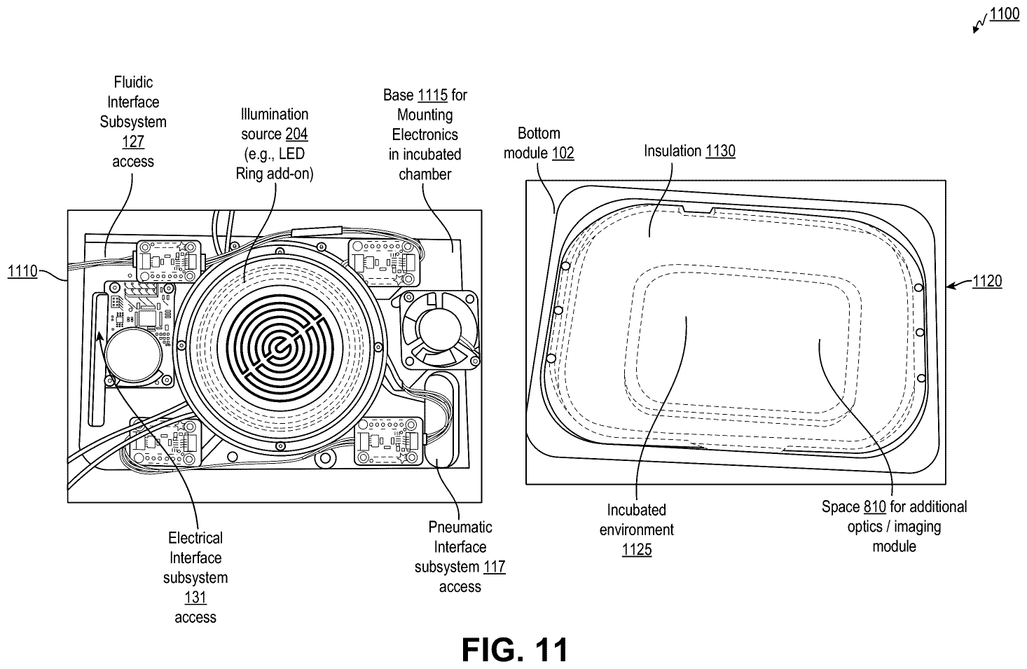

FIG. 11 includes internal views (e.g., view 1110, view 1120) of internal components of the incubation and imaging system 300. In view 1110, several components of the incubation and imaging system 300 are visible, including an access interface for the pneumatic interface subsystem 117, an access interface for the fluidic interface subsystem 127, the electrical interface subsystem 131, the illumination source 204 (e.g., a light emitting diode (LED) ring add-on), and a base 1115 (e.g., printed circuit board (PCB) or other circuit board) for mounting electronics in the incubated chamber (e.g., incubated chamber 139, incubation environment 815, incubated environment 1125). The view 1110 can represent an example of an added-on optical subsystem (with a ring LED illumination source 204) and an example layout of the fluidic interface subsystem 127 access, the electrical interface subsystem 131 access, and the pneumatic interface subsystem 117 access.

In view 1120, several components of the incubation and imaging system 300 are visible, including the bottom module 102, the space 810 for additional optics and/or imaging module(s), the incubated environment 1125 (e.g., incubated chamber 139, incubation environment 815), and insulation 1130 (e.g., insulating material 122) around the incubated environment 1125. The view 1120 can represent an example design of the bottom module 102, with insulation 1130 (e.g., insulating material 122) and the incubated environment 1125 (e.g., incubated chamber 139). In alternate embodiments, the imaging access port 129 and/or a plurality of such ports is fabricated at the bottom of the incubated environment 1125 (not shown in the view 1120) to allow for optical access to the culture dish 128 received by/in the incubated environment 1125.

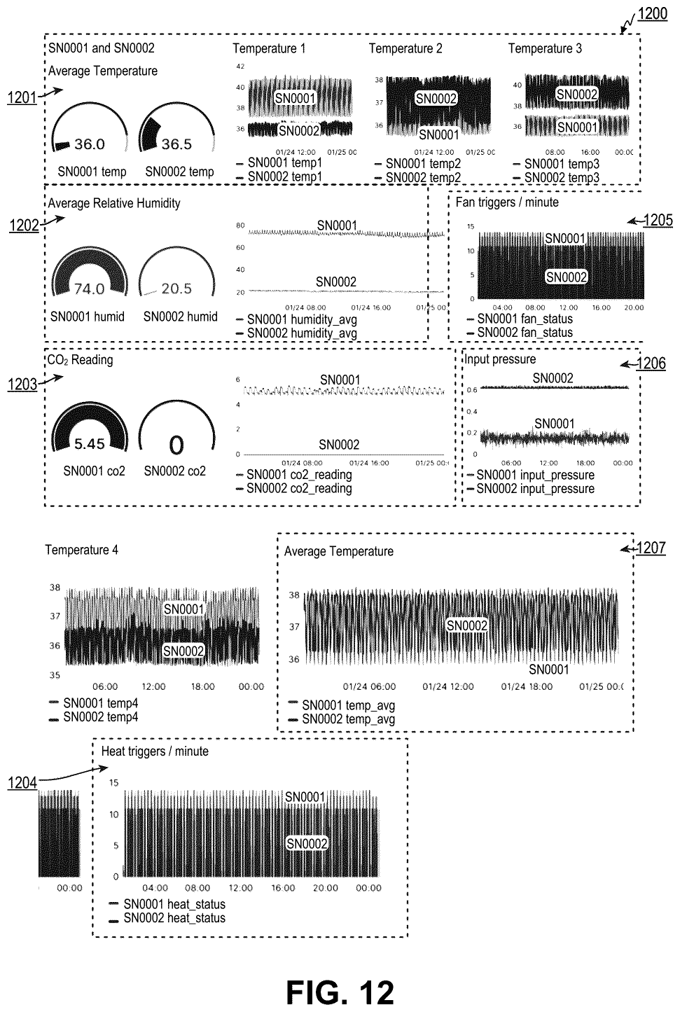

FIG. 12 is a user interface diagram illustrating a dashboard interface 1200 for visualizing data from an incubation and imaging system 300. In the dashboard interface 1200 illustrated in FIG. 12, real-time data from two independently operating incubator subsystems (“SN0001” and “SN0002”) communicated wirelessly (via WIFI) into an on-line cloud database. The database was then polled regularly (at 5 sec intervals), to provide the remote visualization dashboard for the past 1 day. User interface section 1201 and user interface section 1207 shows the individual temperature sensor readings and average temperature reading in the incubated chamber 139, in both test units simultaneously. User interface section 1202 and user interface section 1203 shows the relative humidity and CO₂ gas readings, respectively.

The dashboard interface 1200 provides information to the user, for instance to allow the user to react accordingly to prevent loss of culture growth conditions. In the example illustrated in FIG. 12, the dashboard interface 1200 shows that SN0002 is reporting temperature levels close to the set-point 37 degrees Celsius, however, the humidity and CO₂ levels are compromised. The cloud-service can notify the user of such a scenario via email, text or other forms of information exchange. The user interface section 1204 and the user interface section 1205 provide the user with information regarding the number of times components of the thermal control subsystem 132 were operated. Such information gathered over long-periods of time can be used to predict component failures, such as but not limited to, lack of incubated environment sealing 118 and insulating material 122. The user interface section 1207 shows an example of the pressure sensing reading from the gas cartridges loaded onto the respective test units. In this example, a user can observe that test unit SN0001 pressure has dropped and a cartridge replacement is therefore eminent. In some examples, the layouts for the visualization in the dashboard interface 1200 can display single unit information, additional units and/or provide additional data analysis tools that are configurable by the user.

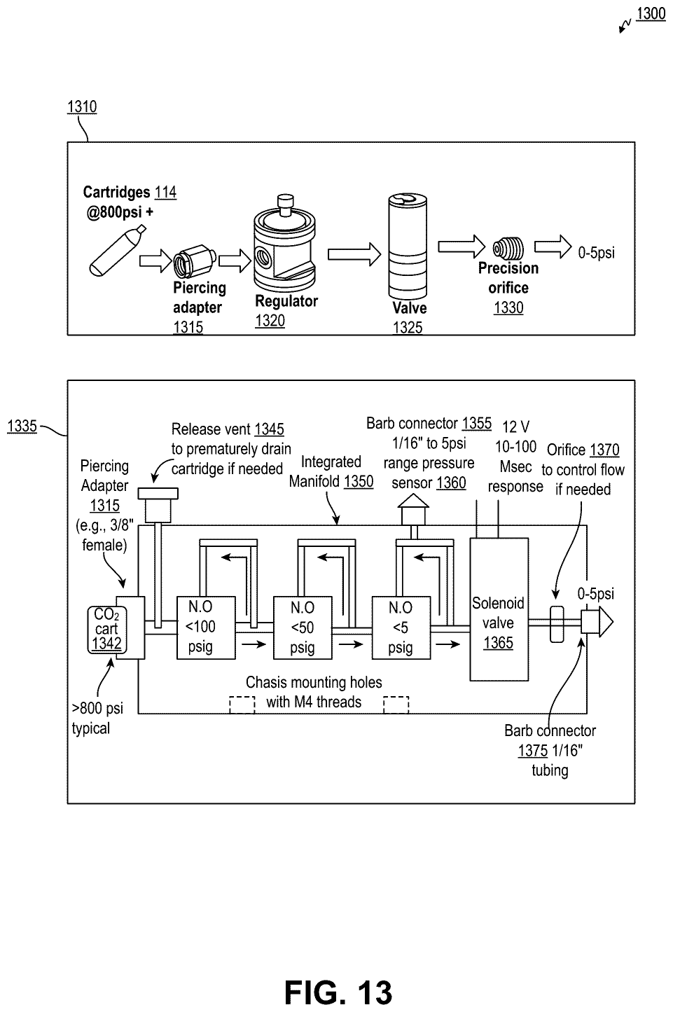

FIG. 13 illustrates two examples of systems to regulate and inject controlled quantities of gas by the gas subsystem 112 in the incubator system 100. In the first diagram 1310, discrete components are assembled together during manufacturing, including a piercing adapter 1315 (that accepts a sealed gas cartridge 114, for instance at gas pressures of at least 800 psi, or even of at least 1200 psi), a pressure regulator 1320 (that drops the cartridge pressure, for instance to <20 psi), a valve 1325 to gate the release of gas, and a precision orifice 1330 to further impede, and thereby control, the gas flow (such as, but not limited to, that provided by a Jewel orifice with an orifice diameter<100 micron).

In the second diagram 1335, various the components of the gas subsystem 112 are integrated into a single integrated pneumatic manifold 1350, allowing for additional compactness of this subsystem. The components, as illustrated in the second diagram 1335, include a carbon dioxide cartridge 1342 (e.g., as an example of the gas cartridge 114), the piercing adapter 1315 (e.g., which may be a ⅜ inch female piercing adapter), a release vent 1345 to prematurely drain the carbon dioxide cartridge 1342 if needed (e.g., if the cell culture environment in the chamber already has over a threshold level of carbon dioxide), a multi-stage regulator (e.g., that drops pressure to below 100 pounds per square inch gauge (psig), then drops pressure to below 50 psig, then drops pressure below 5 psig), the piercing adapter 1315, the pressure regulator 1320 (e.g., with multiple stages), valve 1325, precision orifice 1330, and output connector), a barb connector 1355 (e.g., 1/16 inch) to a 5 psi range pressure sensor 1360 (e.g., measuring whether the pressure is within a threshold range of 5 psi), a solenoid valve 1365 (e.g., as an example of the valve 1325), an orifice 1370 to control flow if needed (e.g., as an example of the precision orifice 1330), an a barb connector 1375 (e.g., 1/16 inch) as an output interface. In some examples, the pressure at the output interface is between zero and 5 psi. In some examples, the gas subsystem 112 can be in a housing that includes chassis mounting holes with screw threads (e.g., M4 threads).

FIG. 14 provides a photographic example of an integrated incubator and imaging system as a single subsystem 1400, without the need for a separate incubator system 100 and imaging system 200. In this embodiment, the top module 1401 (top module 101) and bottom module 1402 (bottom module 102) have subsystems and components as the top module 101 and bottom module 102, described previously. Not shown in FIG. 14 are the insulation layer (e.g., insulating material 122, insulation 1130) and outer chassis.

An optical microscopy subsystem 1406 (e.g., using the OpenFlexure v7 microscope, another microscope, a custom designed microscope system, or a combination thereof) (e.g., as an optical subsystem 205) is added to the bottom module 1402, which includes a camera sensor and an integrated XY stage 1407 (e.g., XY stage 214) to move a culture dish 1404 (e.g., culture dish 128) and a Z-stage (e.g., anti-vibration 215) to move the microscope objective for focusing. The single subsystem 1400 (e.g., and any incubation and imaging system 300) can include actuators coupled to the optical microscopy subsystem 1406 (e.g., the optical subsystem 205), to the XY stage 1407 (e.g., XY stage 214), and/or to the Z-stage (e.g., anti-vibration 215), to move the culture dish 1404 (e.g., culture dish 128) relative to the optical microscopy subsystem 1406 (e.g., the optical subsystem 205) to change focus, to look at different parts of a cell culture, or a combination thereof. Also present in this embodiment is an XYZ stage controller subsystem 1408 (e.g., motion controller subsystem 212) and a video and graphic processor subsystem 1405 (e.g., video & image processor 219).

The various electronic subsystems illustrated in FIG. 14 can be placed inside or outside the bottom module 1402. If placed inside, these components can be sealed using enclosure case(s) to protect the electronics from the high-humidity environment inside the incubator (e.g., incubated chamber 139, incubation environment 815, incubated environment 1125). If placed outside, the cables can be routed using sealed panel interfaces.

In some examples, a system for culture management can include a housing around a chamber (e.g., incubated chamber 139, chamber with incubation environment 815, chamber with incubated environment 1125 of FIG. 11). The chamber receives a culture (e.g., cell and tissue culture dish 128). The system includes at least one chamber environment interface (e.g., fluidic subsystem 109, gas subsystem 112, fluidic interface subsystem 127, pneumatic interface subsystem 117, electrical interface subsystem 131. thermal control subsystem 132, thermal control subsystem 208, motion controller subsystem 212, anti-vibration 215, chamber vent, gas cartridge, pneumatic interface subsystem, fluidic interface subsystem, regulator, valve, constrictor assembly, heating element, solenoid valve with constrictor, fluid pump, check valve, solenoid valve, CO₂ cartridge) that controls a property of an environment within the chamber to maintain the property within a predetermined range. The predetermined range is associated with incubation of the culture. The system includes at least one chamber environment sensor (e.g., system sensor subsystem 108, chamber sensor subsystem 133, system sensor subsystem 210, proximity sensor, pressure sensor, temperature sensor, humidity sensor, gas sensor, gas cartridge pressure sensor, CO₂ sensor, relative humidity sensor, light sensor) that measures the property of the environment within the chamber to verify that the property is within the predetermined range.

In some examples, the property of the environment within the chamber includes at least one of a temperature of the environment within the chamber, a humidity of the environment within the chamber, a pressure of the environment within the chamber, a concentration of a specified gas in the environment within the chamber, a concentration of a specified mixture of gases in the environment within the chamber, a concentration of a specified fluid in the environment within the chamber, a concentration of a specified mixture of fluids in the environment within the chamber, an orientation of the environment within the chamber, or a level of illumination in the environment within the chamber.

In some examples, the least one chamber environment interface includes at least one of a heater that heats the environment within the chamber to increase a temperature of the environment, a cooler that cools the environment within the chamber to decrease the temperature of the environment, a humidifier that humidifies the environment within the chamber to increase a humidity of the environment, a dehumidifier that dehumidifies the environment within the chamber to decrease the humidity of the environment, a pump that adjusts at least one of a pressure in the environment within the chamber or a flow rate of a fluid in the environment within the chamber, a valve that adjusts at least one of the pressure in the environment within the chamber or the flow rate of the fluid in the environment within the chamber a pump that adjusts a concentration of a specified gas in the environment within the chamber, a valve that adjusts the concentration of the specified gas in the environment within the chamber, a pump that adjusts a concentration of a specified mixture of gases in the environment within the chamber, a valve that adjusts the concentration of the specified mixture of gases in the environment within the chamber, a pump that adjusts a concentration of a specified fluid in a culture dish in the environment within the chamber, a valve that adjusts the concentration of the specified fluid in the culture dish in the environment within the chamber, a pump that adjusts a concentration of a specified mixture of fluids in the culture dish in the environment within the chamber, a valve that adjusts the concentration of the specified mixture of fluids in the culture dish in the environment within the chamber, at least one gas container that stores one or more specified gases to provide the one or more specified gases into the environment within the chamber, at least one fluid container that stores one or more specified fluids to provide the one or more specified fluids into the environment within the chamber, a fan that facilitates airflow within the chamber, an actuator that adjusts an orientation of the environment within the chamber, a light source that illuminates the environment within the chamber, or a combination thereof.

In some examples, the least one chamber environment sensor includes at least one of a temperature sensor, a humidity sensor, a gas pressure sensor, a gas concentration sensor, a fluid concentration sensor, a fluid flow rate sensor, an assay sensor (e.g., biomolecular sensors, biomarker sensors), a bioactivity electrical recording sensor, an orientation sensor, a light sensor, or a combination thereof.

In some examples, the system includes at least one sensor (e.g., imaging access port 129, optics subsystem 205, focus detection subsystem 207, illumination source 204) that captures a representation of the culture while the culture is in the chamber. The system includes at least one output interface (e.g., user interface subsystem 103, communication interface 107, user interface subsystem 203, communication interface 218, display, speakers, buttons, haptic feedback actuator) that outputs a notification based on the representation of the culture captured using the at least one sensor. In some examples, the sensor can be referred to as an image sensor, a media capture sensor (e.g., with “media” in this term referring to images, videos, audio, and/or other forms of media content that can be captured as sensor data).

In some examples, the at least one sensor includes at least one image sensor of a camera. The at least one image sensor captures the representation of the culture, wherein the representation of the culture includes at least one of an image of the culture or a video of the culture.

In some examples, the system includes at least one memory storing instructions, and at least one processor (e.g., controller subsystem 106, video & image processor 219). Execution of the instructions by the at least one processor causes the at least one processor to analyze the representation of the culture to detect a condition. The notification is indicative of the condition. In some examples, to analyze the representation of the culture, the at least one processor processes the representation of the culture using a trained machine learning model that detects the condition based on the representation of the culture. In some examples, the execution of the instructions by the at least one processor causes the at least one processor to further train the trained machine learning model based on training data to update the trained machine learning model. The training data includes at least one of the representation of the culture, the condition, the notification, feedback to the notification, or a combination thereof.

In some examples, the at least one output interface includes at least one of a display, a speaker, a haptic feedback actuator, a wired communication interface, or a wireless communication interface.

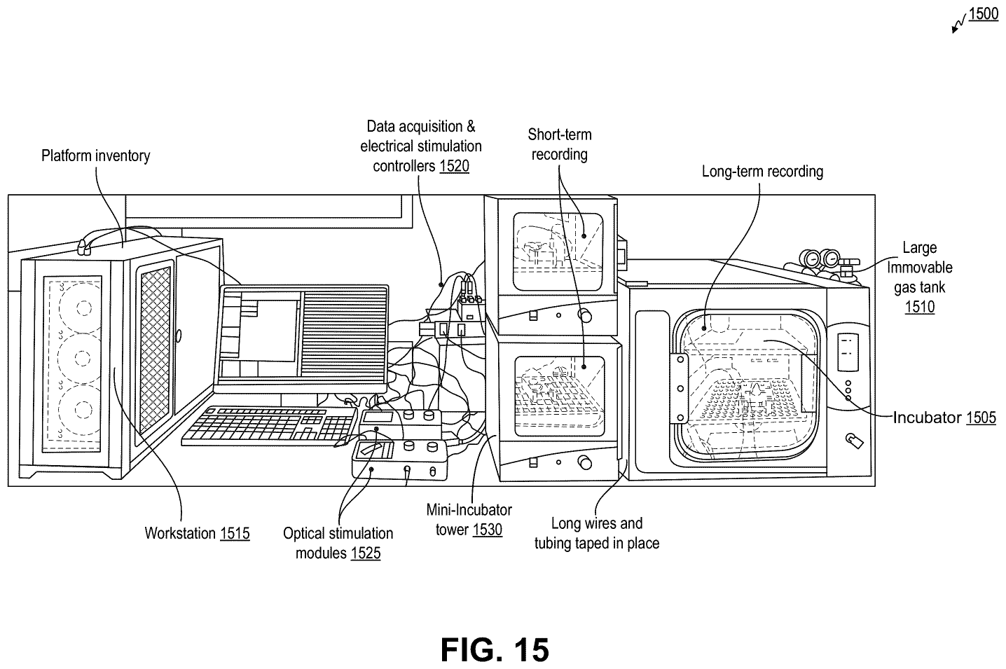

FIG. 15 is a perspective view 1500 of a cell culture setup to perform in-vitro neuronal recordings on microelectrode arrays (MEAs). In some examples, performing microelectrode array (MEA) neuronal recording consists of piecing together many devices in an arrangement such as the one illustrated in the perspective view 1500. These include massive culture incubators (e.g., incubator 1505, mini-incubator tower 1530) (e.g., with large external gas tanks 1510, and powered by AC mains electricity), culture MEA interfaces, MEA pre-amplifiers, data processors (e.g., workstation 1515) for analysis, and fluidic systems for perfusion. They may also include electrical stimulation modules 1520 and optical stimulation modules 1525. A complete setup can easily take up an entire 6 ft workbench or multiple racks, with long optical fibers, fluidic and gas tubing, and long cables susceptible to interference. Thus, one challenge with traditional MEA setups is lack of integration and complex equipment setup. An integrated incubation and imaging system, such as the incubation and imaging system 300, solves issues with disparate non-integrated systems such as these.

Another challenge with traditional MEA setups is optical, electrical and fluidic stimulation. Traditional systems for optical, electrical or pharmacological stimulation do not allow for multi-modal closed-loop stimulation within the context of traditional incubation setups (e.g., perspective view 1500), further increasing equipment system complexity. This severely limits application of machine learning models in health science. An integrated incubation and imaging system, such as the incubation and imaging system 300, solves such issues by allowing for multi-modal closed-loop stimulation, and by allowing use of machine learning models (e.g., ML model(s) 2225).

Another challenge with traditional MEA setups is sub-optimal neuronal growth conditions and high-frequency of contamination. The toxic role that abnormally high oxygen concentration in the atmosphere (20%) as compared to physiological levels (<10%) on neuronal cultures has been well-documented. However, the traditional cell culture incubators control only for CO₂ concentration, not oxygen concentration, hampering neuronal network formation and viability. Long-term neuronal cultures need to remain contamination-free for weeks or months to study neurodevelopmental pathways. Sealed neuronal cultures with breathable membranes and hydrophobic barriers can significantly reduce contamination and hyperosmolality stresses caused by incubator door openings. This can cause the death of cultures and expensive experimental losses. An integrated incubation and imaging system, such as the incubation and imaging system 300, seamlessly integrates of this solution with MEAs dishes at scale, preventing death of cultures and related losses.

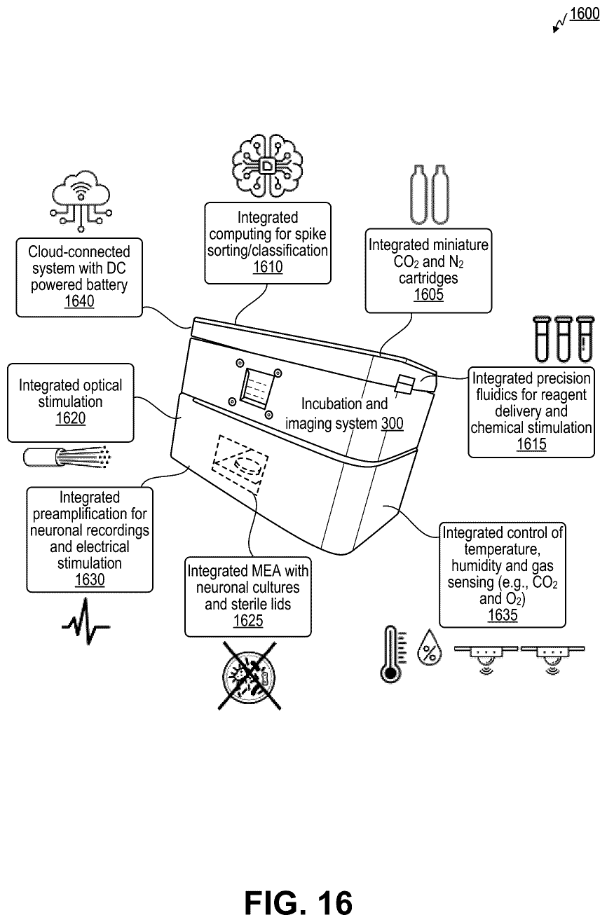

FIG. 16 is a conceptual diagram 1600 illustrating various features of the incubation and imaging system 300. In some examples, the incubation and imaging system 300 is approximately the size of a shoebox. In some examples, the incubation and imaging system 300 can be used as an MEA culture system. In some examples, the incubation and imaging system 300 is battery powered and can be transported when needed without disturbing neuronal recordings.

In some examples, the incubation and imaging system 300 includes, as feature 1605, a hypoxic incubation environment enabled by the use of miniature CO₂ and N₂ gas cartridges (eliminating the need for large gas tanks). The incubation and imaging system 300 can include, as feature 1610, a single-board computer (SBC) for neural spike recording and real-time analysis, chamber environment control, optical and electrical stimulation control, and wireless cloud-interfacing for transmission of experimental data. The incubation and imaging system 300 can include, as feature 1615, miniaturized precision fluidic subsystems for culture media replenishment and reagent delivery to the neuronal culture dishes, while simultaneously recording. The incubation and imaging system 300 can include, as feature 1620, an optical subsystem for optogenetic neuronal stimulation. The incubation and imaging system 300 can include, as feature 1625, easy to load in-vitro MEA dishes with a custom-designed cap that includes breathable membranes to maintain sterility and prevent hyperosmolality stresses. The incubation and imaging system 300 can include, as feature 1630, scalable interfacing to compact pre-amplifier boards that perform all analog signal processing, prior to interfacing directly to the single board computer. The incubation and imaging system 300 can include, as feature 1635, integrated control of temperature, humidity and gas sensing (e.g., CO₂ and O₂). The incubation and imaging system 300 can include, as feature 1640, a cloud-connected system with a DC-powered battery.

The incubation and imaging system 300 is designed to allow neuronal cultures (or other types of cell cultures) to be incubated, monitored, and interacted with robustly for long durations, such as weeks, months, or even years.

The incubation and imaging system 300 greatly improves efficiency and accuracy, prevents cell deaths and damage (e.g., caused by removing cells from incubators to place the cells under microscopes, and doing so for extended periods of time repeatedly), and streamlines the experimental setup for neuroscience researchers. Rather than multiple pieces of equipment to manage, the simplified workflow involves (A) easily loading pre-packaged gas, reagent and media cartridges into the system, (B) opening the system chamber and loading a fresh in-vitro neuronal culture MEA dish with a sealed cap (and in some examples, multiwell MEA plates), (C) closing the chamber, which mechanically and electrically engages the pre-amplifier boards and fluidically engages the culture dish, (D) setting the environmental parameters, stimulation and recording conditions in software, and (E) performing software controlled media replenishment and/or pharmacological treatment without further opening the chamber, while continuously performing long-term recording and analysis using GPU-driven spike sorting with cloud-based remote monitoring. Such a platform will greatly increase the accessibility, standardization, flexibility, and scalability of in-vitro neuronal MEA based experimentation. Examples of this process are illustrated in FIGS. 4-5.

Some examples of the incubation and imaging system 300, or components or subsystems thereof, are illustrated in FIGS. 1, 2, 3, 6, 7, 8, 9, 10, 11, 13, 14, 16, 18, 19, and 21. The incubation and imaging system 300 can be used to control the temperature, humidity and gas levels (e.g., CO₂, O₂, N₂) level inside the incubation environment (e.g., incubated chamber 139, incubation environment 815, incubated environment 1125). The design of the incubation and imaging system 300 includes a top module 101 and a bottom module 102. In some examples, the top module 101 houses electronics, the controller subsystem 106, and sub-systems for gas injection (e.g., gas subsystem 112). Miniature gas cartridges 114, such as CO₂ cartridges (e.g., 16g) are all that is needed to supply the requisite gas into the incubation and imaging system 300. An on-board battery module 105 allows the entire user interface subsystem 103 to be fully portable. The bottom module 102 includes the insulated chamber (e.g., incubated chamber 139, incubation environment 815, incubated environment 1125), sensors (e.g., chamber sensor subsystem 133), and thermal control components (e.g., thermal control subsystem 132) that allow for a system to receive culture dishes 128, such as standard Petri dishes or Society for Biomolecular Screening (SBS)-format well plates, for long-term mammalian cell incubation. In some examples, the incubation and imaging system 300 can be designed to be flexible, for instance to include additional electronic, optical, and/or fluidic interfaces.

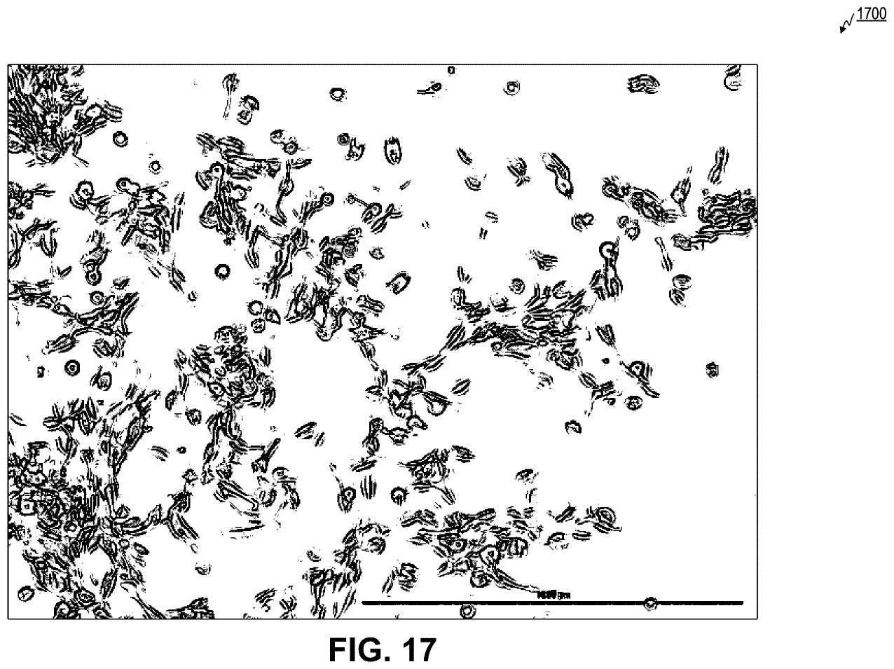

FIG. 17 is an example of a phase contrast image 1700 (modified to be viewable in black and white) of MCF10A wild type cells successfully cultured in the incubator system 100 and imaged. Mammalian MCF (wild type) epithelial cells have been successfully cultured and replicated in the incubation and imaging system 300, and imaged (as in the phase contrast image 1700 of FIG. 17). The cells remain viable for at least over 72 hrs on a single gas cartridge and 12V DC power supply to the incubation and imaging system 300. Similar types of images can be captured using the imaging system 200 and/or the incubation and imaging system 300.

Referring back to FIG. 1—to generate physiological O₂ concentrations below 20%, the design of the incubation and imaging system 300 can include dual gas control (e.g., CO₂ and N₂) using mini-cartridges (e.g., gas cartridge 114), precision regulators (e.g., of the gas subsystem 112), dual solenoid valves (e.g., of the gas subsystem 112), and dual gas-sensors (e.g., of the system sensor subsystem 108). In some examples, nitrogen gas is used to drive oxygen out of the incubation environment (e.g., incubated chamber 139) to reach and maintain physiologically equivalent oxygen levels (<10% O₂). Pressure sensors on each gas channel (e.g., of the system sensor subsystem 108 and/or gas cartridge pressure sensor 625) can monitor cartridge supply levels. The controller subsystem 106 can include a compute module, such as a single-board computer (e.g., NVidia Jetson Orin), which can be integrated into the incubation and imaging system 300 to maintain user defined environment set-points, including gas, temperature, and humidity. The controller subsystem 106 can transfer real-time data to cloud applications (e.g., InfluxData for time-series storage and/or Grafana for data visualization) via its on-board wifi/BLE network interfaces. All hardware modules can be powered by 12V DC supply and/or an on-board Lithium Ion Battery. A custom designed breathable cap (e.g., using 12.7-micron Dupont Teflon® fluorinated ethylene propylene (FEP) film) can be made using 3D-machining and assembly with rubber o-rings. Using FEP-sealed culture dishes has the added benefit of reducing the Relative Humidity levels in the chamber (e.g., ˜65% RH compared to typical >85% RH) due to the membranes' hydrophobic properties. This prevents corrosion, short-circuits and clogs from condensation. To further compact the design, and if needed, to increase space within the chamber, the culture environment can in some examples be sampled using a gas pump and analyzed in the top module 101, rather than in the bottom module 102.

Examples of types of cell cultures that can be incubated and/or imaged using the incubation and imaging system 300 include, by way of example and without limitation, PC12-derived Neuroscreen™-1 cells, primary rodent cortical neurons, primary rodent hippocampal neurons, and Human iPSC derived Glutamatergic neurons. In some examples, for such cells, use of the incubation and imaging system 300 can improve neurite outgrowth and viability compared to experiments performed using traditional incubation systems and imaging systems that are not integrated with one another.

In some examples, custom MEAs can be used with the incubation and imaging system 300 to better enhance experimental flexibility. In some examples, a custom pre-amplifier board consisting of 4×32-channel low-noise amplifier and constant-current stimulator chipsets can be used to interface with the MEA dish that will be placed in the chamber of the incubation and imaging system 300. The custom PCB can use pogo-pins to mechanically and electrically engage with the MEA electrode pads when the user closes the chamber of the incubation and imaging system 300, forming a sound connection to the MEA with no loose hanging electrical cables with stress points. The amplifier digital interfaces will connect via an electrical interface in the top lid of the chamber to the controller subsystem 106 using a dedicated high-speed SPI bus. The operating system (e.g., Linux, Windows, or Mac) on the controller subsystem 106 will allow for installing software packages (e.g., open source or otherwise) for closed-loop neural recording, stimulation, spike sorting and visualization. In some examples, these software packages are part of the incubation and imaging system 300, or are associated with the incubation and imaging system 300.

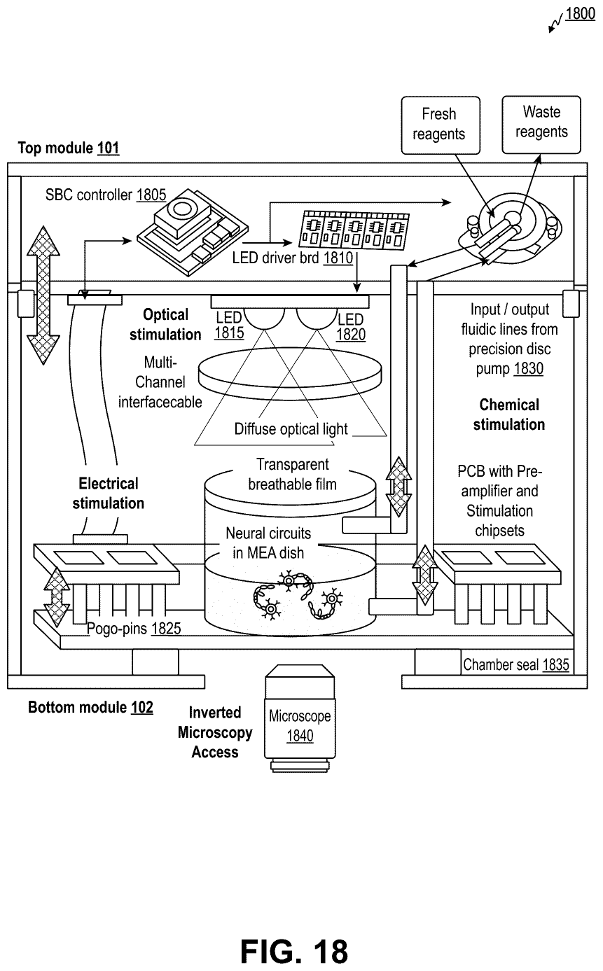

FIG. 18 is a conceptual illustration 1800 of an incubation and imaging system 300 performing multimodal neuronal stimulation and microscopy. The incubation and imaging system 300 illustrated in the conceptual illustration 1800 uses a single-board computer (SBC) controller 1805 as its controller subsystem 106, and also includes a light emitting diode (LED) controller board 1810. In some examples, to deliver optical stimuli, the LED driver board 1810 controls a blue LED 1815 (e.g., at a first wavelength, such as 470 nm,) and an amber LED 1820 (e.g., at a second wavelength, such as 590 nm), along with a 25 mm fused holographic diffuser. To deliver electrical stimuli, the stimulator chipsets of the incubation and imaging system 300 can be programmatically pulsed via pogo-pin interfaces 1825. To deliver chemical stimuli, a miniature, pulsation-free, millisecond response time disc pump 1830 can be fluidically coupled to the neuronal culture dish. A bottom chamber seal 1835 allows for an inverted epi-fluorescence microscope 1840 to perform simultaneous live-cell Ca2+ imaging. In some examples, the microscope 1840 can be the imaging system 200, and/or the imaging subsystem of the incubation and imaging system 300. In some examples, all mechanical, electrical and fluidic interfaces are connected, coupled, activated, and/or actuated automatically in response to the chamber transitioning from an open state to a closed state (see bi-directional arrows with cross-hatched shading in FIG. 18).

In an illustrative examples, primary rat cortical neuronal cultures can be transduced with excitatory and inhibitory optogenetic constructs using vectors AAV-CaMKIIa-hChR2 (H134R)-mCherry and AAV-CaMKIIa-eNpHR 3.0-EYFP. Blue and/or yellow light stimulation can be used to confirm optical control of network firing patterns (e.g. via blue LED 1815 and/or amber LED 1820). Electrical stimulation patterns on the same cultures (e.g., without light) can be tested (e.g., via pogo-pin interfaces 1825) to confirm electrical control of network firing. The spontaneous spiking and bursting activity of Human iPSC-derived Glutamatergic neurons can be modulated by chemical stimulation (e.g., via millisecond response time disc pump 1830) and monitored continuously via the MEA electrodes. The SBC controller 1805 can control media exchanges containing Bicuculline (BIC, 30 μM) to block GABA receptors, D-2-Amino-5-phosphonopentanoic acid (D-APV, 60 μM) to block NMDA receptors, or 6-cyano-7-nitroquinoxaline-2,3-dione (CNQX, 30 μM) to block AMPA receptors. The incubation and imaging system 300 can be used to confirm excitatory or inhibitory effects of the chemical modulation. Inverted epi-fluorescence microscopy can be used to image live neural cultures loaded with Fluo-4-AM calcium indicator. Intracellular fluorescence intensity changes due to calcium influx can be correlated against MEA electrical recordings to confirm Ca2+ imaging functionality. Media exchanges can be performed without opening the chamber (of the incubation and imaging system 300) for cultures for over 1 month.

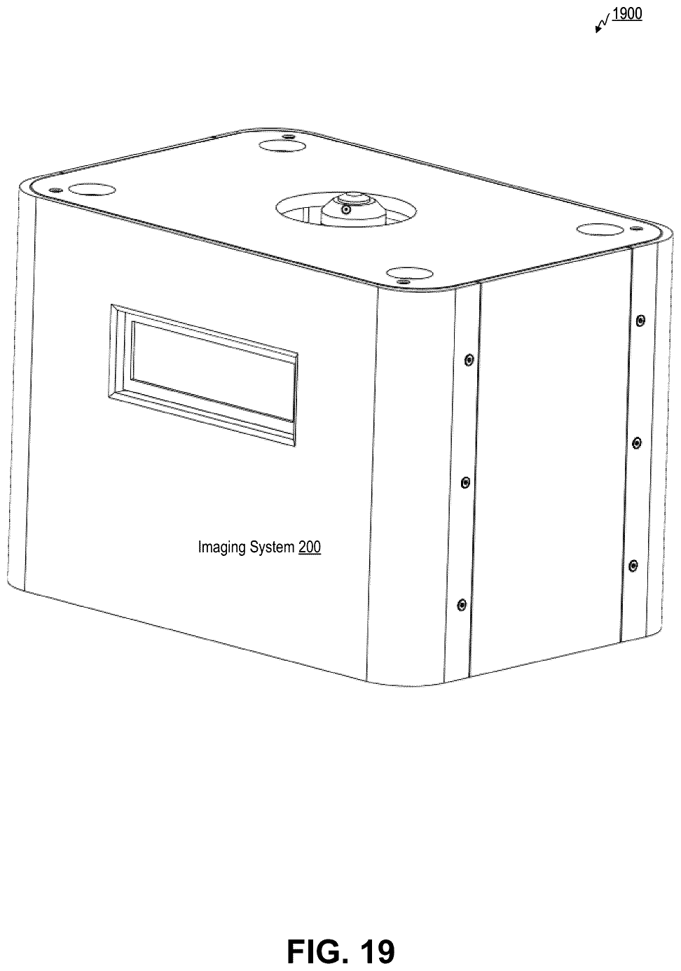

FIG. 19 is a perspective diagram 1900 illustrating an imaging system 200.

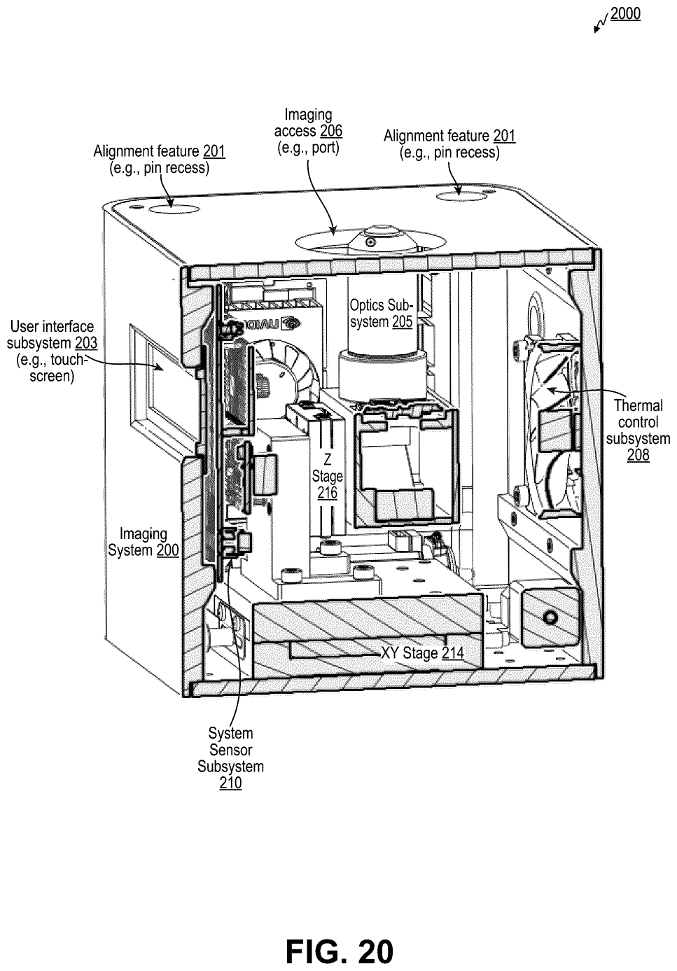

FIG. 20 is a cross-section diagram 2000 illustrating an imaging system 200. Visible within the imaging system 200 of the cross-section diagram 2000 are, for instance, examples of the alignment features 201, the user interface subsystem 203, the optical subsystem 205, the imaging access port 206, the thermal control subsystem 208, the motion controller subsystem 212, the XY stage 214, and the Z-stage 216.

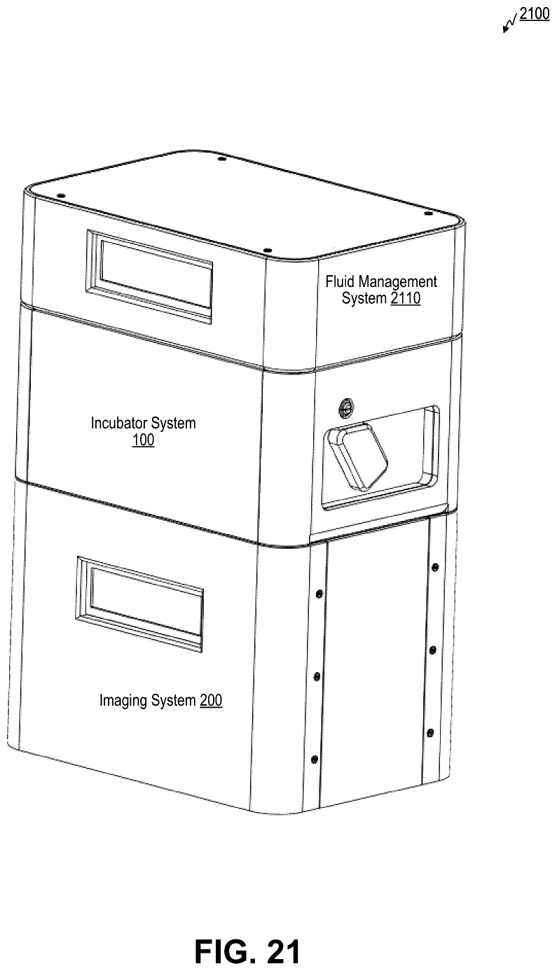

FIG. 21 is a perspective diagram 2100 illustrating an incubation and imaging system 300. The incubation and imaging system 300 includes an incubator system 100 that is detachable coupled to an imaging system 200. The top module 101, the bottom module 102, the user interface subsystem 103, and the user interface subsystem 203 are visible from the exterior of the incubation and imaging system 300. A fluid management system 2110 is also illustrated, as an internal component (or set of components) within the incubator system 100. The fluid management system 2110 can be used by the incubator system 100 to perfuse media with fluids, gases, cell cultures, and/or reagents.

In some examples, the culture dish 128, and/or the culture dish holder 125, may be, or may include, a bioreactor chip and/or biochip. In some examples, incubator system 100 and/or the fluid management system 2110 use pressurized pumps, valves, and/or filters to input fluids (e.g., liquids and/or gases) into the bioreactor (e.g., the culture dish 128 and/or the culture dish holder 125) while also extracting spent medium (e.g., waste, including cellular waste and/or depleted nutrients) to perform perfusion cell culture incubation. In some examples, perfusion can be performed using a predetermined pressure (e.g., 2 PSI). In some examples, under perfusion, the incubator system 100 and/or the fluid management system 2110 adds fresh medium to the bioreactor (e.g., the culture dish 128 and/or the culture dish holder 125) at a specific flow rate, and removes spent medium (e.g., waste) from the bioreactor at the same rate (e.g., within a threshold difference). Filters can ensure that the cells are retained in the bioreactor (e.g., the culture dish 128 and/or the culture dish holder 125) during perfusion, and not extracted with the spend medium. The fresh medium replenishes fluids (e.g., liquids, gases), nutrients, carbon sources, electrolytes, drugs, pharmaceuticals, other materials, or combinations thereof. In some examples, gases provided to the bioreactor (e.g., the culture dish 128 and/or the culture dish holder 125) during perfusion can come from the gas cartridge 114 and/or gas cartridge(s) or reservoir(s) of the fluid management system 2110. In some examples, liquids provided to the bioreactor (e.g., the culture dish 128 and/or the culture dish holder 125) during perfusion can come from a first set of one or more reservoir(s) of the fluidic subsystem 109 and/or of the fluid management system 2110. In some examples, spend medium fluids (e.g., liquids and/or gases) extracted from the bioreactor (e.g., the culture dish 128 and/or the culture dish holder 125) during perfusion can be conveyed to a second set of one or more reservoir(s) of the fluidic subsystem 109 and/or of the gas subsystem 112 and/or the fluid management system 2110.

In some examples, perfusion with culture media can improve quality and/or viability of long-term 3D tissue generation. In some examples, for instance, perfusion for a period of time (e.g., 24 hours) can promote increased spheroid cohesion and collective organization. In some examples, perfusion can present benefits to patch quality. Spheroids are integrated into hydrogels to create patches, but these patches can be degraded by cellular metabolites, leading to low hydrogel strength and a decrease in patch quality. In some examples, perfusion for a period of time (e.g., 24 hours) can reduce the size of hole(s) in hydrogel, for instance reducing a ratio of hole size per spheroid. In some examples, the incubator system 100 and/or the fluid management system 2110 can use valve(s) (e.g., of the fluidic subsystem 109, the gas subsystem 112, the pneumatic interface subsystem 117, and/or the fluidic interface subsystem 127) for fluid line priming, removing bubbles and/or saturating the entire system with media. Because bubbles can impact cell morphology and protein expression, this reduction in bubbles can improve quality and/or viability of cell cultures.

In some examples, the fluid management system 2110 can be a separate subsystem that is detachably coupled to the incubator system 100 and/or the imaging system 200, rather than being internal to the incubator system 100. The subsystems of the incubation and imaging system 300 can be coupled to one another in any order. The imaging system 200 is illustrated as being coupled to the incubator system 100 in an arrangement in which the imaging system 200 is lower (in the direction of gravity) than the incubator system 100. In some examples, the imaging system 200 is instead coupled to the incubator system 100 in an arrangement in which the incubator system 100 is lower (in the direction of gravity) than the imaging system 200. For instance, the components of the incubator system 100 and imaging system 200 can be reversed compared to the arrangements illustrated herein (e.g., with the optical subsystem 205 pointing down through the imaging access port 206 at the bottom of the imaging system 200 and the at the imaging access port 129 at the top of the incubator system 100 at the culture dish 128 toward the top of the incubator system 100). In some examples, an additional subsystem (e.g., the fluid management system 2110) can be coupled to the incubator system 100 and/or the imaging system 200. an additional subsystem (e.g., the fluid management system 2110) can be coupled in between the incubator system 100 and the imaging system 200, and can have two or more imaging access ports (e.g., similar to the imaging access port 129 and/or the imaging access port 206) so that light from the culture dish 128 travels through the imaging access port 129 the imaging access port(s) of the additional subsystem, and the imaging access port 206 before reaching the image sensor of the optical subsystem 205.

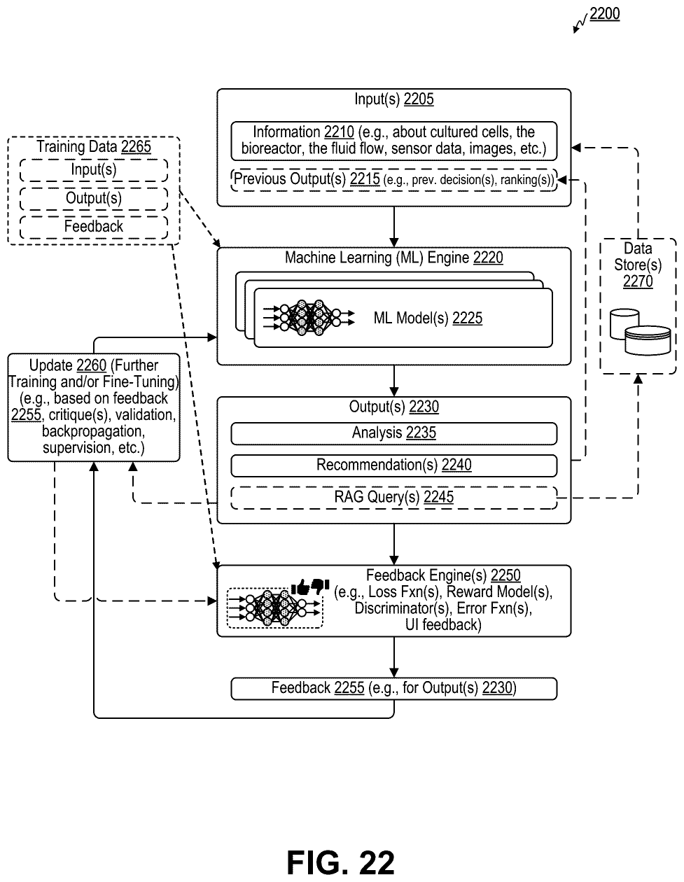

FIG. 22 is a block diagram illustrating an example of a machine learning system 2200 for training, use of, and/or updating of one or more machine learning model(s) 2225 that are used to generate analysis 2235 and/or recommendation(s) 2240. The machine learning (ML) system 2200 includes an ML engine 2220 that generates, trains, uses, and/or updates one or more ML model(s) 2225. In some examples, the incubator systems (e.g., incubator system 100), imaging systems (e.g., imaging system 200), and/or combined incubation and imaging systems (e.g., incubation and imaging system 300) discussed herein include, or have access to, the ML system 2200, the ML system 2200, the ML engine 2220, the ML model(s) 2225, and/or the feedback engine(s) 2250, or vice versa.

The ML model(s) 2225 can include, for instance, one or more neural network(s) (NN(s)), one or more convolutional NN(s) (CNN(s)), one or more time delay NN(s) (TDNN(s)), one or more deep network(s) (DN(s)), one or more autoencoder(s) (AE(s)), one or more variational autoencoder(s) (VAE(s)), one or more deep belief net(s) (DBN(s)), one or more recurrent NN(s) (RNN(s)), one or more generative adversarial network(s) (GAN(s)), one or more conditional GAN(s) (cGAN(s)), one or more feed-forward network(s), one or more network(s) having fully connected layers, one or more support vector machine(s) (SVM(s)), one or more random forest(s) (RF), one or more computer vision (CV) system(s), one or more autoregressive (AR) model(s), one or more Sequence-to-Sequence (Seq2Seq) model(s), one or more large language model(s) (LLM(s)), one or more deep learning system(s), one or more classifier(s), one or more transformer(s), or a combination thereof. In examples where the ML model(s) 2225 include LLMs, the LLMs can include, for instance, a Generative Pre-Trained Transformer (GPT) (e.g., GPT-2, GPT-3, GPT-3.5, GPT-4, etc.), DaVinci or a variant thereof, an LLM using Massachusetts Institute of Technology (MIT)® langchain, Pathways Language Model (PaLM), Large Language Model Meta® AI (LLaMA), Language Model for Dialogue Applications (LaMDA), Bidirectional Encoder Representations from Transformers (BERT), Falcon (e.g., 40B, 7B, 1B), Orca, Phi-1, StableLM, Google® Bard®, Google® Gemini®, DeepSeek® R1, Alibaba® Qwen®, ByteDance® Doubao®, variant(s) of any of the previously-listed LLMs, or a combination thereof.

Within FIG. 22, a graphic representing the ML model(s) 2225 illustrates a set of circles connected to one another. Each of the circles can represent a node, a neuron, a perceptron, a layer, a portion thereof, or a combination thereof. The circles are arranged in columns. The leftmost column of white circles represent an input layer. The rightmost column of white circles represent an output layer. Two columns of shaded circled between the leftmost column of white circles and the rightmost column of white circles each represent hidden layers. An ML model can include more or fewer hidden layers than the two illustrated, but includes at least one hidden layer. In some examples, the layers and/or nodes represent interconnected filters, and information associated with the filters is shared among the different layers with each layer retaining information as the information is processed. The lines between nodes can represent node-to-node interconnections along which information is shared. The lines between nodes can also represent weights (e.g., numeric weights) between nodes, which can be tuned, updated, added, and/or removed as the ML model(s) 2225 are trained and/or updated. In some cases, certain nodes (e.g., nodes of a hidden layer) can transform the information of each input node by applying activation functions (e.g., filters) to this information, for instance applying convolutional functions, downscaling, upscaling, data transformation, and/or any other suitable functions.

In some examples, the ML model(s) 2225 can include a feed-forward network, in which case there are no feedback connections where outputs of the network are fed back into itself. In some cases, the ML model(s) 2225 can include a recurrent neural network, which can have loops that allow information to be carried across nodes while reading in input. In some cases, the network can include a convolutional neural network, which may not link every node in one layer to every other node in the next layer.

One or more input(s) 2205 can be provided to the ML model(s) 2225. The ML model(s) 2225 can be trained by the ML engine 2220 (e.g., based on training data 2265) to generate one or more output(s) 2230. In some examples, the input(s) 2205 include information 2210. The information 2210 can include, for instance, information about cultured cells, about the incubator system(s), about the imaging system(s), about fluid(s) and/or reagent(s) and/or gas(es) to be provided to incubated chamber 139, about waste products received from the incubated chamber 139, sensor data from one or more sensors (e.g., of the incubator system 100 and/or the imaging system 200), image data from one or more cameras (e.g., of the imaging system 200), or a combination thereof. In some examples, the input(s) 2205 can include prompt(s) (e.g., to an LLM). In some examples, the input(s) 2205 can include information retrieved from data store(s) 2270, for instance via retrieval augmented generation (RAG) (e.g., via RAG query(s) 745). In some examples, the input(s) 2205 can include prompt(s) that are modified and/or enhanced using information retrieved from data store(s) 2270, for instance via retrieval augmented generation (RAG) (e.g., via RAG query(s) 745).

The output(s) 2230 that ML model(s) 2225 generate by processing the input(s) 2205 (e.g., the information 2210 and/or the previous output(s) 2215) can include analysis 2235, recommendation(s) 2240, and/or RAG query(s) 2245. The analysis 2235 can include, for instance, analyses of image(s) (e.g., from the imaging system 200), analyses of sample(s) (e.g., from a sampling fluid port for extraction of samples from the incubated chamber 139), analyses of sensor data (e.g., from the sensors of the of the incubator system 100 and/or the imaging system 200), or a combination thereof. In some examples, the analysis 2235 can include feature extraction, feature detection, feature recognition, feature tracking, object detection, object recognition and/or object tracking analyses (e.g., from images captured by the imaging system 200), for instance to track how different cells and/or materials (e.g., pharmaceuticals, drugs, infections) (e.g., with or without luminescence via luminescent markers) move throughout the incubated chamber 139. In some examples, the analysis 2235 can include mass spectrometry analyses.

The recommendation(s) 2240 can include, for instance, recommendation(s) to modify the fluid(s), the gas(es), the cell(s), the reagent(s) (e.g., pharmaceuticals, drugs, markers), and/or other materials provided to the incubated chamber 139 (e.g., via an input fluid port of the fluidic subsystem 109 and/or gas subsystem 112), recommendation(s) to adjust a temperature (e.g., via the thermal control subsystem 132), recommendation(s) to adjust a humidity (e.g., via the humidity wick 126), recommendation(s) to adjust a gas level (e.g., of CO₂, O₂, N₂, or another gas) (e.g., via the gas subsystem 112), recommendation(s) to actuate a valve (e.g., of the fluidic subsystem 109, the gas subsystem 112, the pneumatic interface subsystem 117, the fluidic interface subsystem 127, the check valve 130, the relief valve 142) to switch the valve from one state to another state (e.g., between an open state and a closed state), recommendation(s) to capture image(s) (e.g., using the imaging system 200), recommendation(s) to adjust lighting (e.g., via the illumination source 204), to adjust pH (e.g., via the fluidic subsystem 109, the gas subsystem 112, the pneumatic interface subsystem 117, and/or the fluidic interface subsystem 127), or a combination thereof. In some examples, the recommendation(s) 2240 can be output to a user (e.g., via the user interface subsystem 103 and/or the imaging system 200 and or a cloud-based interface), for instance to allow the user to decide whether to implement the recommended actions, and to allow the user to implement the recommended actions themselves. In some examples, the recommendation(s) 2240 can be output to a system or subsystem (e.g., any of the subsystems and/or components of the incubator system 100, any of the subsystems and/or components of the imaging system 200, any of the subsystems and/or components of the incubator system 300, or a combination thereof) that can automatically implement the recommended actions, for instance by actuating actuators that change the state of a valve, by adjusting an illumination level of the illumination source 204, by causing a camera (e.g., imaging system 200) to capture an image and/or video, by adjusting temperature and/or humidity and/or gas composition and/or liquid composition and/or pH and/or another characteristic, or a combination thereof.

The ML model(s) 2225 can generate the each of the output(s) 2230 based on the information 2210, information from the data store(s) 2270, and/or other types of input(s) 2205 (e.g., previous output(s) 2215).

In some examples, the ML model(s) 2225 can identify something in the input(s) 2205 about which the data store(s) 2270 include additional information, and can fashion at least one query (e.g., the RAG query(s) 2245) for the data store(s) 2270 to retrieve the additional information from the data store(s) 2270. For instance, if the information 2210 references a specific model of device, the RAG query(s) 2245 can include one or more queries of the data store(s) 2270 for additional information about the specific model of device, for instance to retrieve its components, configurations, settings, firmware updates, ranges of optimal operating parameters (e.g., temperature, clock speed, and so forth), or a combination thereof. If the information 2210 references a specific type of cell or drug, the RAG query(s) 2245 can include one or more queries of the data store(s) 2270 for additional information about the specific type of cell or drug, for instance to retrieve its optimal temperatures, humidity levels, acidity levels (e.g., pH), light levels, or a combination thereof. The additional information retrieved from the data store(s) 2270 using the RAG query(s) 2245 can be used as part of the input(s) 2205 (e.g., as part of the information 2210 and/or part of the previous output(s) 2215) for further passes of data processing by the ML model(s) 2225.

In some examples, certain output(s) 2230 (e.g., the analysis 2235, the recommendation(s) 2240, the RAG query(s) 2245) can be used as part of the input(s) 2205 to the ML model(s) 2225 (e.g., as part of previous output(s) 2215) for identifying other output(s) 2230 (e.g., the analysis 2235, the recommendation(s) 2240, the RAG query(s) 2245). For instance, in an illustrative example, the analysis 2235 can be processed, as previous output(s) 2215, by the ML model(s) 2225 to generate the recommendation(s) 2240, the RAG query(s) 2245, and/or other output(s) 2230. In some examples, at least some of the previous output(s) 2215 in the input(s) 2205 represent previously-identified instances of some of the output(s) 2230 that are input into the ML model(s) 2225 to generate other types of the output(s) 2230. In some examples, based on receipt of the input(s) 2205, the ML model(s) 2225 can select the output(s) 2230 from a list of possible outputs, for instance by ranking the list of possible outputs by likelihood, probability, and/or confidence based on the input(s) 2205. In some examples, based on receipt of the input(s) 2205, the ML model(s) 2225 can identify the output(s) 2230 at least in part using generative artificial intelligence (AI) content generation techniques, for instance using an LLM to generate custom text and/or graphics identifying the output(s) 2230. In some examples, the LLM-based output(s) 2230 are conversationally responsive to a prompt in the input(s) 2205 (e.g., in the information 2210 and/or in the previous output(s) 2215).

In some examples, the ML system repeats the process illustrated in FIG. 22 multiple times to generate the output(s) 2230 in multiple passes, using some of the output(s) 2230 from earlier passes as some of the input(s) 2205 in later passes (e.g., as some of the previous output(s) 2215). For instance, in a first illustrative example, in a first pass, the ML model(s) 2225 can identify the analysis 2235 based on input of the information 2210 into the ML model(s) 2225. In a second pass, the ML model(s) 2225 can identify the recommendation(s) 2240 based on input of the information 2210 and the previous output(s) 2215 (that includes the analysis 2235 from the first pass) into the ML model(s) 2225.

In some examples, the ML system includes one or more feedback engine(s) 2250 that generate and/or provide feedback 2255 about the output(s) 2230. In some examples, the feedback 2255 indicates how well the output(s) 2230 align to corresponding expected output(s), how well the output(s) 2230 serve their intended purpose, or a combination thereof. In some examples, the feedback engine(s) 2250 include loss function(s), reward model(s) (e.g., other ML model(s) that are used to score the output(s) 2230), discriminator(s), error function(s) (e.g., in back-propagation), user interface feedback received via a user interface from a user, or a combination thereof. In some examples, the feedback 2255 can include one or more alignment analysis that score a level of alignment between the output(s) 2230 and the expected output(s) and/or intended purpose.

The ML engine 2220 of the ML system can update (further train) the ML model(s) 2225 based on the feedback 2255 to perform an update 2260 (e.g., further training) of the ML model(s) 2225 based on the feedback 2255. In some examples, the feedback 2255 includes positive feedback, for instance indicating that the output(s) 2230 closely align with expected output(s) and/or that the output(s) 2230 serve their intended purpose. In some examples, the feedback 2255 includes negative feedback, for instance indicating a mismatch between the output(s) 2230 and the expected output(s), and/or that the output(s) 2230 do not serve their intended purpose. For instance, high amounts of loss and/or error (e.g., exceeding a threshold) can be interpreted as negative feedback, while low amounts of loss and/or error (e.g., less than a threshold) can be interpreted as positive feedback. Similarly, high amounts of alignment (e.g., exceeding a threshold) can be interpreted as positive feedback, while low amounts of alignment (e.g., less than a threshold) can be interpreted as negative feedback.

In response to positive feedback in the feedback 2255, the ML engine 2220 can perform the update 2260 to update the ML model(s) 2225 to strengthen and/or reinforce weights (and/or connections and/or hyperparameters) associated with generation of the output(s) 2230 to encourage the ML engine 2220 to generate similar output(s) 2230 given similar input(s) 2205. In this way, the update 2260 can improve the ML model(s) 2225 itself by improving the accuracy of the ML model(s) 2225 in generating output(s) 2230 that are similarly accurate given similar input(s) 2205. In response to negative feedback in the feedback 2255, the ML engine 2220 can perform the update 2260 to update the ML model(s) 2225 to weaken and/or remove weights (and/or connections and/or hyperparameters) associated with generation of the output(s) 2230 to discourage the ML engine 2220 from generating similar output(s) 2230 given similar input(s) 2205. In this way, the update 2260 can improve the ML model(s) 2225 itself by improving the accuracy of the ML model(s) 2225 in generating output(s) 2230 are more accurate given similar input(s) 2205. In some examples, for instance, the update 2260 can improve the accuracy of the ML model(s) 2225 in generating output(s) 2230 by reducing false positive(s) and/or false negative(s) in the output(s) 2230.

For instance, here, if the analysis 2235 and/or recommendation(s) 2240 are used in an experiment, and the experiment is successful, the success of the experiment can be interpreted as feedback 2255 that is positive (e.g., positive feedback). For instance, here, if the analysis 2235 and/or recommendation(s) 2240 are used in an experiment, and the experiment fails or is unsuccessful, the failure or lack of success of the experiment can be interpreted as feedback 2255 that is negative (e.g., negative feedback). Either way, the update 2260 can improve the machine learning system 2200 and the overall system by improving the consistency with which the experiment is successful.

In some examples, the ML engine 2220 can also perform an initial training of the ML model(s) 2225 before the ML model(s) 2225 are used to generate the output(s) 2230 based on the input(s) 2205. During the initial training, the ML engine 2220 can train the ML model(s) 2225 based on training data 2265. In some examples, the training data 2265 includes examples of input(s) (of any input types discussed with respect to the input(s) 2205), output(s) (of any output types discussed with respect to the output(s) 2230), and/or feedback (of any feedback types discussed with respect to the feedback 2255). In some cases, positive feedback in the training data 2265 can be used to perform positive training, to encourage the ML model(s) 2225 to generate output(s) similar to the output(s) in the training data given input of the corresponding input(s) in the training data. In some cases, negative feedback in the training data 2265 can be used to perform negative training, to discourage the ML model(s) 2225 from generate output(s) similar to the output(s) in the training data given input of the corresponding input(s) in the training data. In some examples, the training of the ML model(s) 2225 (e.g., the initial training with the training data 2265, update(s) 2260 based on the feedback 2255, and/or other modification(s)) can include fine-tuning of the ML model(s) 2225, retraining of the ML model(s) 2225, or a combination thereof.

In some examples, the ML model(s) 2225 can include an ensemble of multiple ML models, and the ML engine 2220 can curate and manage the ML model(s) 2225 in the ensemble. The ensemble can include ML model(s) 2225 that are different from one another to produce different respective outputs, which the ML engine 2220 can average (e.g., mean, median, and/or mode) to identify the output(s) 2230. In some examples, the ML engine 2220 can calculate the standard deviation of the respective outputs of the different ML model(s) 2225 in the ensemble to identify a level of confidence in the output(s) 2230. In some examples, the standard deviation can have an inverse relationship with confidence. For instance, if the respective outputs of the different ML model(s) 2225 are very different from one another (and thus have a high standard deviation above a threshold), the confidence that the output(s) 2230 are accurate may be low (e.g., below a threshold). On the other hand, if the respective outputs of the different ML model(s) 2225 are equal or very similar to one another (and thus have a low standard deviation below a threshold), the confidence that the output(s) 2230 are accurate may be high (e.g., above a threshold). In some examples, different ML models(s) 2225 in the ensemble can include different types of models. For instance, in some examples, an ensemble can include a NN and a SVM that are both trained to process the input(s) 2205 to generate at least a subset of the output(s) 2230. In some examples, the ensemble may include different ML model(s) 2225 that are trained to process different inputs of the input(s) 2205 and/or to generate different outputs of the output(s) 2230. For instance, in some examples, a first model (or set of models) can process the input(s) 2205 to generate the analysis 2235, a second model (or set of models) can process the input(s) 2205 to generate the recommendation(s) 2240, and a third model (or set of models) can process the input(s) 2205 to generate the RAG query(s) 2245. In some examples, the ML engine 2220 can choose specific ML model(s) 2225 to be included in the ensemble because the chosen ML model(s) 2225 are effective at accurately processing particular types of input(s) 2205, are effective at accurately generating particular types of output(s) 2230, are generally accurate, process input(s) 2205 quickly, generate output(s) 2230 quickly, are computationally efficient, have higher or lower degrees of uncertainty than other models in the ensemble, or a combination thereof.

In some examples, one or more of the ML model(s) 2225 can be initialized with weights, connections, and/or hyperparameters that are selected randomly. This can be referred to as random initialization. These weights, connections, and/or hyperparameters are modified over time through training (e.g., initial training with the training data 2265 and/or update(s) 2260 based on the feedback 2255), but the random initialization can still influence the way the ML model(s) 2225 process data, and thus can still cause different ML model(s) 2225 (with different random initializations) to produce different output(s) 2230. Thus, in some examples, different ML model(s) 2225 in an ensemble can have different random initializations.

As an ML model (of the ML model(s) 2225) is trained (e.g., along the initial training with the training data 2265, update(s) 2260 based on the feedback 2255, and/or other modification(s)), different versions of the ML model at different stages of training can be referred to as checkpoints. In some examples, after each new update to a model (e.g., update 2260) generates a new checkpoint for the model, the ML engine 2220 tests the new checkpoint (e.g., against testing data and/or validation data where the correct output(s) are known) to identify whether the new checkpoint improves over older checkpoints or not, and/or if the new checkpoint introduces new errors (e.g., false positive(s) and/or false negative(s)). This testing can be referred to as checkpoint benchmark scoring. In some examples, in checkpoint benchmark scoring, the ML engine 2220 produces a benchmark score for one or more checkpoint(s) of one or more ML model(s) 2225, and keeps the checkpoint(s) that have the best (e.g., highest or lowest) benchmark scores in the ensemble. In some examples, if a new checkpoint is worse than an older checkpoint, the ML engine 2220 can revert to the older checkpoint. The benchmark score for a can represent a level of accuracy of the checkpoint and/or number of errors (e.g., false positive or false negative) by the checkpoint during the testing (e.g., against the testing data and/or the validation data). In some examples, an ensemble of the ML model(s) 2225 can include multiple checkpoints of the same ML model.

In some examples, the ML model(s) 2225 can be modified, either through the initial training (with the training data 2265), an update 2260 based on the feedback 2255, or another modification to introduce randomness, variability, and/or uncertainty into an ensemble of the ML model(s) 2225. In some examples, such modification(s) to the ML model(s) 2225 can include dropout (e.g., Monte Carlo dropout), in which one or more weights or connections are selected at random and removed. In some examples, dropout can also be performed during inference, for instance to modify the output(s) 2230 generated by the ML model(s) 2225. The term Bayesian Machine Learning (BML) can refer to random dropout, random initialization, and/or other randomization-based modifications to the ML model(s) 2225. In some examples, the modification(s) to the ML model(s) 2225 can include a hyperparameter search and/or adjustment of hyperparameters. The hyperparameter search can involve training and/or updating different ML models 2225 with different values for hyperparameters and evaluating the relative performance of the ML models 2225 (e.g., against (e.g., against testing data and/or validation data where the correct output(s) are known) to identify which of the ML models 2225 performs best. Hyperparameters can include, for instance, temperature (e.g., influencing level creativity and/or randomness), top P (e.g., influencing level creativity and/or randomness), frequency penalty (e.g., to prevent repetitive language between one of the output(s) 2230 and another), presence penalty (e.g., to encourage the ML model(s) 2225 to introduce new data in the output(s) 2230), other parameters or settings, or a combination thereof.

In some examples, the ML engine 2220 can perform retrieval-augmented generation (RAG) using the model(s) 2225. For instance, in some examples, the ML engine 2220 can pre-process the input(s) 2205 by retrieving additional information from one or more data store(s) 2270 (e.g., any of the databases and/or other data structures discussed herein) and using the additional information to enhance the input(s) 2205 before the input(s) 2205 are processed by the ML model(s) 2225 to generate the output(s) 2230. For instance, in some examples, the enhanced versions of the input(s) 2205 can include the additional information that the ML engine 2220 retrieved from the from one or more data store(s) 2270. In some examples, the machine learning system 2200 can retrieve the additional information from one or more data store(s) 2270 by querying the data store(s) 2270 using RAG query(s) 2245 generated by the ML model(s) 2225 (or extracted from the input(s) 2205 using the ML model(s) 2225). In some examples, this RAG process provides the ML model(s) 2225 with more relevant information, allowing the ML model(s) 2225 to generate more accurate and/or personalized output(s) 2230.

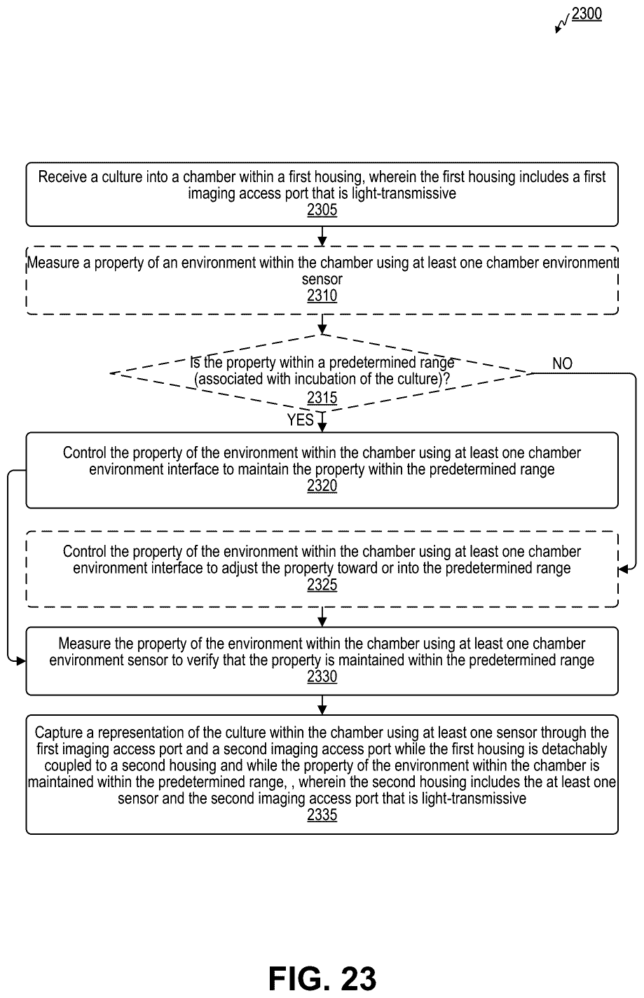

FIG. 23 is a flow diagram illustrating a process 2300 for cell incubation and imaging. The process 2300 is performed by a cell culture processing system. The cell culture processing system may be, and/or may include, the incubator system 100, the imaging system 200, the incubation and imaging system 300, a system that performs the process 400, a system that performs the process 500, any of the components illustrated in FIGS. 6-11, a system that uses the dashboard interface 1200, any of the components illustrated in FIGS. 13-16, a system that captures the phase contrast image 1700, any of the components illustrated in FIG. 18, the machine learning system 2200, the ML engine 2220, the ML model(s) 2225, the feedback engine(s) 2250, the computing system 2400, a processor that executes instructions stored in a memory, a processor that executes instructions stored in a non-transitory computer-readable medium, a system, an apparatus, or a combination thereof.

At operation 2305, the cell culture processing system is configured to, and can, receive a culture (e.g., culture dish 128) into a chamber (e.g., incubated chamber 139, incubation environment 815, incubated environment 1125) within a first housing (e.g., insulating material 122, housing of the incubator system 100, housing of the incubation subsystem of the incubation and imaging system 300). The chamber includes a first imaging access port (e.g., imaging access port 129) that is light-transmissive (e.g., transparent, translucent). In some examples, the first imaging access port is a lens. In some examples, the first imaging access port is made of glass or plastic (e.g., acrylic). In some examples, the first imaging access port is an orifice, an aperture, and/or a hole within the first housing, with a reversible environmental seal developed between the culture dish and the housing (e.g. chamber seal 140).

At operation 2310, in some examples, the cell culture processing system is configured to, and can, measure a property of an environment within the chamber using at least one chamber environment sensor (e.g., chamber sensor subsystem 133). At operation 2315, in some examples, the cell culture processing system is configured to, and can, check if the property is within a predetermined range that is associated with incubation of the culture. If the property is within the predetermined range, operation 2315 is followed by operation 2320. If the property is outside of the predetermined range, operation 2315 is followed by operation 2325.

At operation 2320, the cell culture processing system is configured to, and can, control the property of the environment within the chamber using at least one chamber environment interface (e.g., fluidic subsystem 109, gas subsystem 112, pneumatic interface subsystem 117, fluidic interface subsystem 127) to maintain the property within the predetermined range. In some examples, the at least one chamber environment interface provides at least a fluid (e.g., a gas via the gas subsystem 112 and/or the 117, and/or a liquid via the fluidic subsystem 109 and/or the fluidic interface subsystem 127) to the chamber to maintain the property within the predetermined range.

At operation 2325, in some examples, the cell culture processing system is configured to, and can, control the property of the environment within the chamber using at least one chamber environment interface (e.g., fluidic subsystem 109, gas subsystem 112, pneumatic interface subsystem 117, fluidic interface subsystem 127) to adjust the property toward or into the predetermined range. In some examples, the at least one chamber environment interface provides at least a fluid (e.g., a gas via the gas subsystem 112 and/or the 117, and/or a liquid via the fluidic subsystem 109 and/or the fluidic interface subsystem 127) to the chamber to adjust the property toward or into the predetermined range.

At operation 2330, the cell culture processing system is configured to, and can, measure the property of the environment within the chamber using at least one chamber environment sensor (e.g., chamber sensor subsystem 133) to verify that the property is maintained within the predetermined range.

At operation 2335, the cell culture processing system is configured to, and can, capture a representation (e.g., an image, a video, or another type of sensor data) of the culture within the chamber using at least one sensor through the first imaging access port and a second imaging access port while the first housing is detachably coupled to a second housing and while the property of the environment within the chamber is maintained within the predetermined range. The second housing (e.g., of the imaging system 200 and/or the imaging subsystem of the incubation and imaging system 300) includes the at least one sensor and the second imaging access port that is light-transmissive (e.g., transparent, translucent). In some examples, the first imaging access port is a lens. In some examples, the first imaging access port is made of glass or plastic (e.g., acrylic). For instance, light from the cell culture is conveyed through both the first imaging access port and the second imaging access port before reaching the at least one sensor. In some examples, at least one of the first imaging access port and/or the second imaging access port may be an orifice, an aperture, and/or a hole within the respective housing.

In some examples, the first housing is detachably coupled to the second housing using fastener(s) on the first housing and/or the second housing. For instance the fastener(s) can include latches, magnets, pins that go into recesses, screws, clips, slide rails, elastic, seals, hook and loop fasteners, stud fasteners, other types of fasteners, or combinations thereof. In some examples, the alignment features 137 may represent examples of the fasteners on the incubator system 100. In some examples, the alignment features 201 may represent examples of the fasteners on the imaging system 200. In some examples, alignment features 137 and the alignment features 201 may be detachably coupled together (e.g., detachably attached, connected, secured, and/or fastened) in the incubation and imaging system 300 as illustrated in FIG. 3. In some examples, the fastener(s) on the incubator system 100 and/or on the imaging system 200 may include electrical coupling interfaces (e.g., plugs, ports, and/or other types of electrical contacts). In some examples, electrical power can be conveyed across the electrical coupling interfaces of the fasteners while the first housing (e.g., incubator system 100) is coupled to the second housing (e.g., imaging system 200). In some examples, data (e.g., sensor data, instructions to the at least one chamber environment interface, instructions to the at least one sensor) can be conveyed across the electrical coupling interfaces of the fasteners while the first housing (e.g., incubator system 100) is coupled to the second housing (e.g., imaging system 200). In some examples, a third housing (e.g., of the fluid management system 2110) is also coupled to the first housing (e.g., incubator system 100) and/or the second housing (e.g., imaging system 200) to form the incubation and imaging system 300 (e.g., as illustrated in the perspective diagram 2100), for instance using any of the types of fasteners (e.g., with or without electrical coupling interfaces) discussed herein. In some examples, the electrical coupling interfaces can also be used to convey power and/or data between the fluid management system 2110 and the incubator system 100, and/or between the fluid management system 2110 and the imaging system 200.

In some examples, the at least one sensor can include an image sensor of a camera (e.g., of the imaging system 200 and/or the incubation and imaging system 300). In some examples, the at least one sensor can include an electrical sensor that captures electrical recordings (e.g., neuronal recording) of electrical characteristics (e.g., voltage, wattage, current, electrical impedance), which may be important in some cases (e.g., where the cell cultures include neurons or other types of cells that perform electrical activity). In some examples, the at least one sensor can be referred to as a media sensor, for instance where it captures images, videos, audio, and/or other types of media content. In some examples, the at least one sensor can be referred to as a characterization sensor, in that the sensor's data represents a characterization of the status and/or behavior of the cell culture.

In some examples, the property of the environment within the chamber includes at least one of a temperature of the environment within the chamber, a humidity of the environment within the chamber, a pressure of the environment within the chamber, a pH of the environment within the chamber, a fluidic flow rate of a fluid within the chamber, an orientation of the environment within the chamber, a level of illumination in the environment within the chamber, a concentration of a specified gas in the environment within the chamber, a concentration of a specified mixture of gases in the environment within the chamber, a concentration of a specified liquid in the environment within the chamber, a concentration of a specified mixture of liquids in the environment within the chamber, concentration of a reagent in the environment within the chamber, or a combination thereof. Note that the fluid (of operation 2320 and/or operation 2325) can be an example of the specified gas, the specified mixture of gases, the specified liquid, the specified mixture of liquids, the reagent, or a combination thereof.

In some examples, the least one chamber environment interface includes at least one of a heater that heats the environment within the chamber to increase a temperature of the environment, a cooler that cools the environment within the chamber to decrease the temperature of the environment, a humidifier that humidifies the environment within the chamber to increase a humidity of the environment, a dehumidifier that dehumidifies the environment within the chamber to decrease the humidity of the environment, a pump that adjusts a pressure in the environment within the chamber, a valve that adjusts the pressure in the environment within the chamber, a pump that adjusts a concentration of a specified gas in the environment within the chamber, a valve that adjusts the concentration of the specified gas in the environment within the chamber, a pump that adjusts a concentration of a specified mixture of gases in the environment within the chamber, a valve that adjusts the concentration of the specified mixture of gases in the environment within the chamber, a pump that adjusts a concentration of a specified fluid in a culture dish in the environment within the chamber, a valve that adjusts the concentration of the specified fluid in the culture dish in the environment within the chamber, a pump that adjusts a concentration of a specified mixture of fluids in the culture dish in the environment within the chamber, a valve that adjusts the concentration of the specified mixture of fluids in the culture dish in the environment within the chamber, at least one gas container that stores one or more specified gases to provide the one or more specified gases into the environment within the chamber, at least one fluid container that stores one or more specified fluids to provide the one or more specified fluids into the environment within the chamber, at least one gas container that stores one or more specified gases to provide the one or more specified gases into the environment within the chamber, at least one fluid container that stores one or more specified fluids to provide the one or more specified fluids into the environment within the chamber, a fan, an actuator that adjusts an orientation of the environment within the chamber, or a light source that illuminates the environment within the chamber.

In some examples, the least one chamber environment interface includes at least one of a pump or a valve, and wherein the least one chamber environment interface adjusts at least one of a concentration of a specified gas in the environment within the chamber, a concentration of a specified mixture of gases in the environment within the chamber, a concentration of a specified fluid in a culture dish in the environment within the chamber, a concentration of a specified mixture of fluids in the culture dish in the environment within the chamber, a concentration of a specified reagent in the culture dish in the environment within the chamber.

In some examples, the least one chamber environment sensor includes at least one of a temperature sensor, a humidity sensor, a gas pressure sensor, a gas concentration sensor, a fluid concentration sensor, an assay sensor, a bioactivity electrical recording sensor, an orientation sensor, an image sensor, a light sensor, or a combination thereof.

In some examples, the cell culture processing system is configured to, and can, outputs a notification based on the representation of the culture using an output interface (e.g., user interface subsystem 103, communication interface 107, user interface subsystem 203, communication interface 218). In some examples, the output interface includes at least one of a display, a speaker, a haptic feedback actuator, a wired communication interface, or a wireless communication interface.

In some examples, the representation is an image, and the image is captured using an image sensor. In some examples, the image is a still image. In some examples, the image is a video frame of a video.

In some examples, the cell culture processing system is configured to, and can, analyze the representation of the culture to detect a condition, and output a notification associated with the condition. In some examples, to analyze the representation of the culture, the at least one processor processes the representation of the culture using a trained machine learning model (e.g., ML model(s) 2225) that detects the condition based on the representation of the culture (e.g., analysis 2235, recommendation(s) 2240). In some examples, the cell culture processing system is configured to, and can, update (e.g., update 2260) the trained machine learning model based on training data to improve an accuracy of the trained machine learning model. The training data includes at least one of the representation, the condition, the notification, or feedback (e.g., feedback 2255) associated with the notification.

In some examples, the property is temperature, and controlling the property (in operation 2320 and/or operation 2325) includes activating a heater, a fan, a cooling element (e.g., a cooler), or another component of the thermal control subsystem 132. In some examples, the predetermined range is a range around a predetermined temperature. In some examples, the predetermined temperature is 37 degrees Celsius. In some examples, the predetermined temperature is different from 37 degrees Celsius, for instance because certain types of cells grow best at different temperatures other than 37 degrees Celsius. For instance, in some examples, the predetermined temperature is 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., a temperature in between any two of the above-listed temperatures, or another temperature.

In some examples, the property is a specific concentration of a specific gas, such as CO₂. In some examples, the predetermined range is a range around a predetermined concentration. In some examples, the predetermined concentration is 5% CO₂.

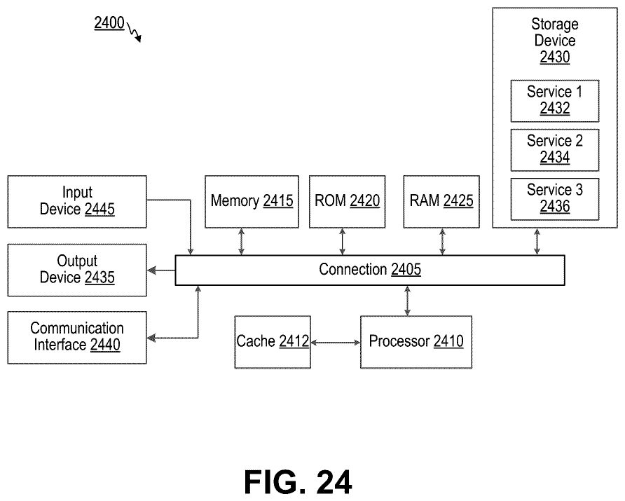

FIG. 24 illustrates an exemplary computing system 2400 that may be used to implement some aspects of the technology. For example, any of the computing devices, computing systems, network devices, network systems, servers, and/or arrangements of circuitry described herein may include at least one computing system 2400, or may include at least one component of the computer system 2400 identified in FIG. 24. The computing system 2400 of FIG. 24 includes one or more processors 2410 and memory 2420. Each of the processor(s) 2410 may refer to one or more processors, controllers, microcontrollers, central processing units (CPUs), graphics processing units (GPUs), arithmetic logic units (ALUs), accelerated processing units (APUs), digital signal processors (DSPs), application specific integrated circuits (ASICs), field-programmable gate arrays (FPGAs), or combinations thereof. Each of the processor(s) 2410 may include one or more cores, either integrated onto a single chip or spread across multiple chips connected or coupled together. Memory 2420 stores, in part, instructions and data for execution by processor 2410. Memory 2420 can store the executable code when in operation. The computing system 2400 of FIG. 24 further includes a mass storage device 2430, portable storage device(s) 2440 (e.g., drive(s) and/or other storage media), output devices 2450, user input devices 2460, a display system 2470 (e.g., graphics display), and peripheral device(s) 2480.

The components shown in FIG. 24 are depicted as being connected via a single bus 2490. However, the components may be connected through one or more data transport means. For example, processor 2410 and memory 2420 may be connected via a local microprocessor bus, and the mass storage device 2430, peripheral device(s) 2480, portable storage device 2440, and display system 2470 may be connected via one or more input/output (I/O) buses.

Mass storage device 2430, which may be implemented with a magnetic disk drive or an optical disk drive, is a non-volatile storage device for storing data and instructions for use by processor 2410. Mass storage device 2430 can store the system software for implementing some aspects of the subject technology for purposes of loading that software into memory 2420.

Portable storage device 2440 operates in conjunction with a portable non-volatile storage medium, such as a floppy disk, compact disk or Digital video disc, to input and output data and code to and from the computer system 2400 of FIG. 24. The system software for implementing aspects of the subject technology may be stored on such a portable medium and input to the computer system 2400 via the portable storage device 2440.

The memory 2420, mass storage device 2430, or portable storage device 2440 may in some cases store sensitive information, such as transaction information, health information, or cryptographic keys, and may in some cases encrypt or decrypt such information with the aid of the processor 2410. The memory 2420, mass storage device 2430, or portable storage device 2440 may in some cases store, at least in part, instructions, executable code, or other data for execution or processing by the processor 2410.

Output devices 2450 may include, for example, communication circuitry for outputting data through wired or wireless means, display circuitry for displaying data via a display screen, audio circuitry for outputting audio via headphones or a speaker, printer circuitry for printing data via a printer, or some combination thereof. The display screen may be any type of display discussed with respect to the display system 2470. The printer may be inkjet, laserjet, thermal, or some combination thereof. In some cases, the output device 2450 (and/or associated circuitry) may allow for transmission of data over an audio jack/plug, a microphone jack/plug, a universal serial bus (USB) port/plug, an Apple® Lightning® port/plug, an Ethernet port/plug, a fiber optic port/plug, a proprietary wired port/plug, a BLUETOOTH® wireless signal transfer, a BLUETOOTH® low energy (BLE) wireless signal transfer, an IBEACON® wireless signal transfer, a radio-frequency identification (RFID) wireless signal transfer, near-field communications (NFC) wireless signal transfer, dedicated short range communication (DSRC) wireless signal transfer, 502.11 Wi-Fi wireless signal transfer, wireless local area network (WLAN) signal transfer, Visible Light Communication (VLC), Worldwide Interoperability for Microwave Access (WiMAX), Infrared (IR) communication wireless signal transfer, Public Switched Telephone Network (PSTN) signal transfer, Integrated Services Digital Network (ISDN) signal transfer, 3G/4G/5G/LTE cellular data network wireless signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof. Output devices 2450 may include any ports, plugs, antennae, wired or wireless transmitters, wired or wireless transceivers, or any other components necessary for or usable to implement the communication types listed above, such as cellular Subscriber Identity Subsystem (SIM) cards.

Input devices 2460 may include circuitry providing a portion of a user interface. Input devices 2460 may include an alpha-numeric keypad, such as a keyboard, for inputting alpha-numeric and other information, or a pointing device, such as a mouse, a trackball, stylus, or cursor direction keys. Input devices 2460 may include touch-sensitive surfaces as well, either integrated with a display as in a touchscreen, or separate from a display as in a trackpad. Touch-sensitive surfaces may in some cases detect localized variable pressure or force detection. In some cases, the input device circuitry may allow for receipt of data over an audio jack, a microphone jack, a universal serial bus (USB) port/plug, an Apple® Lightning® port/plug, an Ethernet port/plug, a fiber optic port/plug, a proprietary wired port/plug, a wired local area network (LAN) port/plug, a BLUETOOTH® wireless signal transfer, a BLUETOOTH® low energy (BLE) wireless signal transfer, an IBEACON® wireless signal transfer, a radio-frequency identification (RFID) wireless signal transfer, near-field communications (NFC) wireless signal transfer, dedicated short range communication (DSRC) wireless signal transfer, 502.11 Wi-Fi wireless signal transfer, wireless local area network (WLAN) signal transfer, Visible Light Communication (VLC), Worldwide Interoperability for Microwave Access (WiMAX), Infrared (IR) communication wireless signal transfer, Public Switched Telephone Network (PSTN) signal transfer, Integrated Services Digital Network (ISDN) signal transfer, 3G/4G/5G/LTE cellular data network wireless signal transfer, personal area network (PAN) signal transfer, wide area network (WAN) signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof. Input devices 2460 may include any ports, plugs, antennae, wired or wireless receivers, wired or wireless transceivers, or any other components necessary for or usable to implement the communication types listed above, such as cellular SIM cards.

Input devices 2460 may include receivers or transceivers used for positioning of the computing system 2400 as well. These may include any of the wired or wireless signal receivers or transceivers. For example, a location of the computing system 2400 can be determined based on signal strength of signals as received at the computing system 2400 from three cellular network towers, a process known as cellular triangulation. Fewer than three cellular network towers can also be used-even one can be used-though the location determined from such data will be less precise (e.g., somewhere within a particular circle for one tower, somewhere along a line or within a relatively small area for two towers) than via triangulation. More than three cellular network towers can also be used, further enhancing the location's accuracy. Similar positioning operations can be performed using proximity beacons, which might use short-range wireless signals such as BLUETOOTH® wireless signals, BLUETOOTH® low energy (BLE) wireless signals, IBEACON® wireless signals, personal area network (PAN) signals, microwave signals, radio wave signals, or other signals discussed above. Similar positioning operations can be performed using wired local area networks (LAN) or wireless local area networks (WLAN) where locations are known of one or more network devices in communication with the computing system 2400 such as a router, modem, switch, hub, bridge, gateway, or repeater. These may also include Global Navigation Satellite System (GNSS) receivers or transceivers that are used to determine a location of the computing system 2400 based on receipt of one or more signals from one or more satellites associated with one or more GNSS systems. GNSS systems include, but are not limited to, the US-based Global Positioning System (GPS), the Russia-based Global Navigation Satellite System (GLONASS), the China-based BeiDou Navigation Satellite System (BDS), and the Europe-based Galileo GNSS. Input devices 2460 may include receivers or transceivers corresponding to one or more of these GNSS systems.

Display system 2470 may include a liquid crystal display (LCD), a plasma display, an organic light-emitting diode (OLED) display, a low-temperature poly-silicon (LTPO) display, an electronic ink or “e-paper” display, a projector-based display, a holographic display, or another suitable display device. Display system 2470 receives textual and graphical information, and processes the information for output to the display device. The display system 2470 may include multiple-touch touchscreen input capabilities, such as capacitive touch detection, resistive touch detection, surface acoustic wave touch detection, or infrared touch detection. Such touchscreen input capabilities may or may not allow for variable pressure or force detection.

Peripheral device(s) 2480 may include any type of computer support device to add additional functionality to the computer system. For example, peripheral device(s) 2480 may include one or more additional output devices of any of the types discussed with respect to output device 2450, one or more additional input devices of any of the types discussed with respect to input device 2460, one or more additional display systems of any of the types discussed with respect to display system 2470, one or more memories or mass storage devices or portable storage devices of any of the types discussed with respect to memory 2420 or mass storage device 2430 or portable storage device 2440, a modem, a router, an antenna, a wired or wireless transceiver, a printer, a bar code scanner, a quick-response (“QR”) code scanner, a magnetic stripe card reader, a integrated circuit chip (ICC) card reader such as a smartcard reader or a EUROPAY®-MASTERCARD®-VISA® (EMV) chip card reader, a near field communication (NFC) reader, a document/image scanner, a visible light camera, a thermal/infrared camera, an ultraviolet-sensitive camera, a night vision camera, a light sensor, a phototransistor, a photoresistor, a thermometer, a thermistor, a battery, a power source, a proximity sensor, a laser rangefinder, a sonar transceiver, a radar transceiver, a lidar transceiver, a network device, a motor, an actuator, a pump, a conveyer belt, a robotic arm, a rotor, a drill, a chemical assay device, or some combination thereof.

The components contained in the computer system 2400 of FIG. 24 can include those typically found in computer systems that may be suitable for use with some aspects of the subject technology and represent a broad category of such computer components that are well known in the art. That said, the computer system 2400 of FIG. 24 can be customized and specialized for the purposes discussed herein and to carry out the various operations discussed herein, with specialized hardware components, specialized arrangements of hardware components, and/or specialized software. Thus, the computer system 2400 of FIG. 24 can be a personal computer, a hand held computing device, a telephone (“smartphone” or otherwise), a mobile computing device, a workstation, a server (on a server rack or otherwise), a minicomputer, a mainframe computer, a tablet computing device, a wearable device (such as a watch, a ring, a pair of glasses, or another type of jewelry or clothing or accessory), a video game console (portable or otherwise), an e-book reader, a media player device (portable or otherwise), a vehicle-based computer, another type of computing device, or some combination thereof. The computer system 2400 may in some cases be a virtual computer system executed by another computer system. The computer can also include different bus configurations, networked platforms, multi-processor platforms, etc. Various operating systems can be used including Unix®, Linux®, FreeBSD®, FreeNAS®, pfSense®, Windows®, Apple® Macintosh OS® (“MacOS®”), Palm OS®, Google® Android®, Google® Chrome OS®, Chromium® OS®, OPENSTEP®, XNU®, Darwin®, Apple® iOS®, Apple® tvOS®, Apple® watchOS®, Apple® audioOS®, Amazon® Fire OS®, Amazon® Kindle OS®, variants of any of these, other suitable operating systems, or combinations thereof. The computer system 2400 may also use a Basic Input/Output System (BIOS) or Unified Extensible Firmware Interface (UEFI) as a layer upon which the operating system(s) are run.

In some cases, the computer system 2400 may be part of a multi-computer system that uses multiple computer systems 2400, each for one or more specific tasks or purposes. For example, the multi-computer system may include multiple computer systems 2400 communicatively coupled together via at least one of a personal area network (PAN), a local area network (LAN), a wireless local area network (WLAN), a municipal area network (MAN), a wide area network (WAN), or some combination thereof. The multi-computer system may further include multiple computer systems 2400 from different networks communicatively coupled together via the internet (also known as a “distributed” system).

Some aspects of the subject technology may be implemented in an application that may be operable using a variety of devices. Non-transitory computer-readable storage media refer to any medium or media that participate in providing instructions to a central processing unit (CPU) for execution and that may be used in the memory 2420, the mass storage device 2430, the portable storage device 2440, or some combination thereof. Such media can take many forms, including, but not limited to, non-volatile and volatile media such as optical or magnetic disks and dynamic memory, respectively. Some forms of non-transitory computer-readable media include, for example, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity subsystem (SIM) card, a mini/micro/nano/pico SIM card, another integrated circuit (IC) chip/card, random access memory (RAM), static RAM (SRAM), dynamic RAM (DRAM), read-only memory (ROM), programmable read-only memory (PROM), erasable programmable read-only memory (EPROM), electrically erasable programmable read-only memory (EEPROM), flash EPROM (FLASHEPROM), cache memory (L1/L2/L3/L4/L5/L7), resistive random-access memory (RRAM/ReRAM), phase change memory (PCM), spin transfer torque RAM (STT-RAM), another memory chip or cartridge, or a combination thereof.

Various forms of transmission media may be involved in carrying one or more sequences of one or more instructions to a processor 2410 for execution. A bus 2490 carries the data to system RAM or another memory 2420, from which a processor 2410 retrieves and executes the instructions. The instructions received by system RAM or another memory 2420 can optionally be stored on a fixed disk (mass storage device 2430/portable storage device 2440) either before or after execution by processor 2410. Various forms of storage may likewise be implemented as well as the necessary network interfaces and network topologies to implement the same.

While various flow diagrams and block diagrams provided and described above may show a particular order of operations performed by some embodiments of the subject technology, it should be understood that such order is exemplary. Alternative embodiments may perform the operations in a different order, combine certain operations, overlap certain operations, or some combination thereof. It should be understood that unless disclosed otherwise, any process illustrated in any flow diagram herein or otherwise illustrated or described herein may be performed by a machine, mechanism, and/or computing system 2400 discussed herein, and may be performed automatically (e.g., in response to one or more triggers/conditions described herein), autonomously, semi-autonomously (e.g., based on received instructions), or a combination thereof. Furthermore, any action described herein as occurring in response to one or more particular triggers/conditions should be understood to optionally occur automatically response to the one or more particular triggers/conditions.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

Illustrative aspects of the disclosure include:
Aspect 1. A system for culture processing, the system comprising: a first housing around a chamber, wherein the chamber receives a culture, and wherein the first housing includes a first imaging access port that is light-transmissive; at least one chamber environment interface that controls a property of an environment within the chamber to maintain the property within a predetermined range, wherein the predetermined range is associated with incubation of the culture; at least one chamber environment sensor that measures the property of the environment within the chamber to verify that the property is maintained within the predetermined range; and at least one sensor within a second housing, wherein the second housing includes a second imaging access port that is light-transmissive, and wherein the at least one sensor captures a representation of the culture within the chamber through the first imaging access port and the second imaging access port while the first housing is detachably coupled to the second housing and while the property of the environment within the chamber is maintained within the predetermined range.
Aspect 2. The system of Aspect 1, wherein the property of the environment within the chamber includes at least one of a temperature of the environment within the chamber, a humidity of the environment within the chamber, a pressure of the environment within the chamber, a pH of the environment within the chamber, a fluidic flow rate of a fluid within the chamber, an orientation of the environment within the chamber, or a level of illumination in the environment within the chamber.
Aspect 3. The system of any one of Aspects 1 to 2, wherein the property of the environment within the chamber includes a concentration of a specified gas in the environment within the chamber, a concentration of a specified mixture of gases in the environment within the chamber, a concentration of a specified liquid in the environment within the chamber, a concentration of a specified mixture of liquids in the environment within the chamber, or concentration of a reagent in the environment within the chamber.
Aspect 4. The system of any one of Aspects 1 to 3, wherein the least one chamber environment interface includes at least one of a heater that heats the environment within the chamber to increase a temperature of the environment, a cooler that cools the environment within the chamber to decrease the temperature of the environment, a humidifier that humidifies the environment within the chamber to increase a humidity of the environment, a dehumidifier that dehumidifies the environment within the chamber to decrease the humidity of the environment, a pump that adjusts at least one of a pressure in the environment within the chamber or a flow rate of a fluid in the environment within the chamber, a valve that adjusts at least one of the pressure in the environment within the chamber or the flow rate of the fluid in the environment within the chamber, a fan that facilitates airflow within the chamber, an actuator that adjusts an orientation of the environment within the chamber, or a light source that illuminates the environment within the chamber.
Aspect 5. The system of any one of Aspects 1 to 4, wherein the least one chamber environment interface includes at least one of a pump or a valve, and wherein the least one chamber environment interface adjusts at least one of a concentration of a specified gas in the environment within the chamber, a concentration of a specified mixture of gases in the environment within the chamber, a concentration of a specified fluid in a culture dish in the environment within the chamber, a concentration of a specified mixture of fluids in the culture dish in the environment within the chamber, a concentration of a specified reagent in the culture dish in the environment within the chamber.
Aspect 6. The system of any one of Aspects 1 to 5, wherein the least one chamber environment sensor includes at least one of a temperature sensor, a humidity sensor, a gas pressure sensor, a gas concentration sensor, a fluid concentration sensor, a fluid flow rate sensor, an assay sensor, a bioactivity electrical recording sensor, an orientation sensor, or a light sensor.
Aspect 7. The system of any one of Aspects 1 to 6, further comprising: at least one output interface that outputs a notification based on the representation of the culture.
Aspect 8. The system of Aspect 7, wherein the at least one output interface includes at least one of a display, a speaker, a haptic feedback actuator, a wired communication interface, or a wireless communication interface.
Aspect 9. The system of any one of Aspects 1 to 8, wherein the at least one sensor includes at least one image sensor, and wherein the representation of the culture includes at least one image of the culture.
Aspect 10. The system of any one of Aspects 1 to 9, further comprising: at least one memory storing instructions; and at least one processor, wherein execution of the instructions by the at least one processor causes the at least one processor to: analyze the representation of the culture to detect a condition; and output a notification that is indicative of the condition.
Aspect 11. The system of Aspect 10, wherein, to analyze the representation of the culture, the at least one processor processes the representation of the culture using a trained machine learning model that detects the condition based on the representation of the culture.
Aspect 12. The system of Aspect 11, wherein the execution of the instructions by the at least one processor causes the at least one processor to: update the trained machine learning model based on training data to improve an accuracy of the trained machine learning model, wherein the training data includes at least one of the representation, the condition, the notification, or feedback associated with the notification.
Aspect 13. A method of culture processing, the method comprising: receiving a culture into a chamber within a first housing, wherein the first housing includes a first imaging access port that is light-transmissive; controlling a property of an environment within the chamber using at least one chamber environment interface to maintain the property within a predetermined range, wherein the predetermined range is associated with incubation of the culture; measuring the property of the environment within the chamber using at least one chamber environment sensor to verify that the property is maintained within the predetermined range; and capturing a representation of the culture within the chamber using at least one sensor through the first imaging access port and a second imaging access port while the first housing is detachably coupled to a second housing and while the property of the environment within the chamber is maintained within the predetermined range, wherein the second housing includes the at least one sensor and the second imaging access port that is light-transmissive.
Aspect 14. The method of Aspect 13, wherein the property of the environment within the chamber includes at least one of a temperature of the environment within the chamber, a humidity of the environment within the chamber, a pressure of the environment within the chamber, a pH of the environment within the chamber, a fluidic flow rate of a fluid within the chamber, an orientation of the environment within the chamber, or a level of illumination in the environment within the chamber.
Aspect 15. The method of any one of Aspects 13 to 14, wherein the property of the environment within the chamber includes a concentration of a specified gas in the environment within the chamber, a concentration of a specified mixture of gases in the environment within the chamber, a concentration of a specified liquid in the environment within the chamber, a concentration of a specified mixture of liquids in the environment within the chamber, or concentration of a reagent in the environment within the chamber.
Aspect 16. The method of any one of Aspects 13 to 15, wherein the least one chamber environment interface includes at least one of a heater that heats the environment within the chamber to increase a temperature of the environment, a cooler that cools the environment within the chamber to decrease the temperature of the environment, a humidifier that humidifies the environment within the chamber to increase a humidity of the environment, a dehumidifier that dehumidifies the environment within the chamber to decrease the humidity of the environment, a pump that adjusts at least one of a pressure in the environment within the chamber or a flow rate of a fluid in the environment within the chamber, a valve that adjusts at least one of the pressure in the environment within the chamber or the flow rate of the fluid in the environment within the chamber, a fan that facilitates airflow within the chamber, an actuator that adjusts an orientation of the environment within the chamber, or a light source that illuminates the environment within the chamber.
Aspect 17. The method of any one of Aspects 13 to 16, wherein the least one chamber environment interface includes at least one of a pump or a valve, and wherein the least one chamber environment interface adjusts at least one of a concentration of a specified gas in the environment within the chamber, a concentration of a specified mixture of gases in the environment within the chamber, a concentration of a specified fluid in a culture dish in the environment within the chamber, a concentration of a specified mixture of fluids in the culture dish in the environment within the chamber, a concentration of a specified reagent in the culture dish in the environment within the chamber.
Aspect 18. The method of any one of Aspects 13 to 17, wherein the least one chamber environment sensor includes at least one of a temperature sensor, a humidity sensor, a gas pressure sensor, a gas concentration sensor, a fluid concentration sensor, a fluid flow rate sensor, an assay sensor, a bioactivity electrical recording sensor, an orientation sensor, or a light sensor.
Aspect 19. The method of any one of Aspects 13 to 18, further comprising: outputting, through at least one output interface, a notification based on the representation of the culture.
Aspect 20. The method of Aspect 19, wherein the at least one output interface includes at least one of a display, a speaker, a haptic feedback actuator, a wired communication interface, or a wireless communication interface.
Aspect 21. The method of any one of Aspects 13 to 20, wherein the representation is captured by at least one image sensor, and wherein the representation of the culture includes at least one image of the culture.
Aspect 22. The method of any one of Aspects 13 to 21, further comprising: analyzing the representation of the culture to detect a condition; and outputting a notification that is indicative of the condition.
Aspect 23. The method of Aspect 22, wherein, analyzing the representation of the culture includes processing the representation of the culture using a trained machine learning model that detects the condition based on the representation of the culture.
Aspect 24. The method of Aspect 23, further comprising: updating the trained machine learning model based on training data to improve an accuracy of the trained machine learning model, wherein the training data includes at least one of the representation, the condition, the notification, or feedback associated with the notification.
Aspect 25. A non-transitory computer-readable medium having stored thereon instructions that, when executed by one or more processors, cause the one or more processors to perform operations according to any of Aspects 1 to 24.
Aspect 26. An apparatus for culture processing, the apparatus comprising one or more means for performing operations according to any of Aspects 1 to 24.

What is claimed is:

1. A system for culture processing, the system comprising:
a first housing around a chamber, wherein the chamber receives a culture, and wherein the first housing includes a first imaging access port that is light-transmissive;
at least one chamber environment interface that controls a property of an environment within the chamber to maintain the property within a predetermined range, wherein the predetermined range is associated with incubation of the culture;
at least one chamber environment sensor that measures the property of the environment within the chamber to verify that the property is maintained within the predetermined range; and
at least one sensor within a second housing, wherein the second housing includes a second imaging access port that is light-transmissive, and wherein the at least one sensor captures a representation of the culture within the chamber through the first imaging access port and the second imaging access port while the first housing is detachably coupled to the second housing and while the property of the environment within the chamber is maintained within the predetermined range.

2. The system of claim 1, wherein the property of the environment within the chamber includes at least one of a temperature of the environment within the chamber, a humidity of the environment within the chamber, a pressure of the environment within the chamber, a pH of the environment within the chamber, a fluidic flow rate of a fluid within the chamber, an orientation of the environment within the chamber, or a level of illumination in the environment within the chamber.

3. The system of claim 1, wherein the property of the environment within the chamber includes a concentration of a specified gas in the environment within the chamber, a concentration of a specified mixture of gases in the environment within the chamber, a concentration of a specified liquid in the environment within the chamber, a concentration of a specified mixture of liquids in the environment within the chamber, or concentration of a reagent in the environment within the chamber.

4. The system of claim 1, wherein the least one chamber environment interface includes at least one of a heater that heats the environment within the chamber to increase a temperature of the environment, a cooler that cools the environment within the chamber to decrease the temperature of the environment, a humidifier that humidifies the environment within the chamber to increase a humidity of the environment, a dehumidifier that dehumidifies the environment within the chamber to decrease the humidity of the environment, a pump that adjusts at least one of a pressure in the environment within the chamber or a flow rate of a fluid in the environment within the chamber, a valve that adjusts at least one of the pressure in the environment within the chamber or the flow rate of the fluid in the environment within the chamber, a fan that facilitates airflow within the chamber, an actuator that adjusts an orientation of the environment within the chamber, or a light source that illuminates the environment within the chamber.

5. The system of claim 1, wherein the least one chamber environment interface includes at least one of a pump or a valve, and wherein the least one chamber environment interface adjusts at least one of a concentration of a specified gas in the environment within the chamber, a concentration of a specified mixture of gases in the environment within the chamber, a concentration of a specified fluid in a culture dish in the environment within the chamber, a concentration of a specified mixture of fluids in the culture dish in the environment within the chamber, a concentration of a specified reagent in the culture dish in the environment within the chamber.

6. The system of claim 1, wherein the least one chamber environment sensor includes at least one of a temperature sensor, a humidity sensor, a gas pressure sensor, a gas concentration sensor, a fluid concentration sensor, a fluid flow rate sensor, an assay sensor, a bioactivity electrical recording sensor, an orientation sensor, or a light sensor.

7. The system of claim 1, further comprising:
at least one output interface that outputs a notification based on the representation of the culture.

8. The system of claim 7, wherein the at least one output interface includes at least one of a display, a speaker, a haptic feedback actuator, a wired communication interface, or a wireless communication interface.

9. The system of claim 1, wherein the at least one sensor includes at least one image sensor, and wherein the representation of the culture includes at least one image of the culture.

10. The system of claim 1, further comprising:
at least one memory storing instructions; and
at least one processor, wherein execution of the instructions by the at least one processor causes the at least one processor to:
analyze the representation of the culture to detect a condition; and
output a notification that is indicative of the condition.

11. The system of claim 10, wherein, to analyze the representation of the culture, the at least one processor processes the representation of the culture using a trained machine learning model that detects the condition based on the representation of the culture.

12. The system of claim 11, wherein the execution of the instructions by the at least one processor causes the at least one processor to:
update the trained machine learning model based on training data to improve an accuracy of the trained machine learning model, wherein the training data includes at least one of the representation, the condition, the notification, or feedback associated with the notification.

13. A method of culture processing, the method comprising:
receiving a culture into a chamber within a first housing, wherein the first housing includes a first imaging access port that is light-transmissive;
controlling a property of an environment within the chamber using at least one chamber environment interface to maintain the property within a predetermined range, wherein the predetermined range is associated with incubation of the culture;
measuring the property of the environment within the chamber using at least one chamber environment sensor to verify that the property is maintained within the predetermined range; and
capturing a representation of the culture within the chamber using at least one sensor through the first imaging access port and a second imaging access port while the first housing is detachably coupled to a second housing and while the property of the environment within the chamber is maintained within the predetermined range, wherein the second housing includes the at least one sensor and the second imaging access port that is light-transmissive.

14. The method of claim 13, wherein the property of the environment within the chamber includes at least one of a temperature of the environment within the chamber, a humidity of the environment within the chamber, a pressure of the environment within the chamber, a pH of the environment within the chamber, a fluidic flow rate of a fluid within the chamber, an orientation of the environment within the chamber, or a level of illumination in the environment within the chamber.

15. The method of claim 13, wherein the property of the environment within the chamber includes a concentration of a specified gas in the environment within the chamber, a concentration of a specified mixture of gases in the environment within the chamber, a concentration of a specified liquid in the environment within the chamber, a concentration of a specified mixture of liquids in the environment within the chamber, or concentration of a reagent in the environment within the chamber.

16. The method of claim 13, wherein the least one chamber environment interface includes at least one of a heater that heats the environment within the chamber to increase a temperature of the environment, a cooler that cools the environment within the chamber to decrease the temperature of the environment, a humidifier that humidifies the environment within the chamber to increase a humidity of the environment, a dehumidifier that dehumidifies the environment within the chamber to decrease the humidity of the environment, a pump that adjusts at least one of a pressure in the environment within the chamber or a flow rate of a fluid in the environment within the chamber, a valve that adjusts at least one of the pressure in the environment within the chamber or the flow rate of the fluid in the environment within the chamber, a fan that facilitates airflow within the chamber, an actuator that adjusts an orientation of the environment within the chamber, or a light source that illuminates the environment within the chamber.

17. The method of claim 13, wherein the least one chamber environment interface includes at least one of a pump or a valve, and wherein the least one chamber environment interface adjusts at least one of a concentration of a specified gas in the environment within the chamber, a concentration of a specified mixture of gases in the environment within the chamber, a concentration of a specified fluid in a culture dish in the environment within the chamber, a concentration of a specified mixture of fluids in the culture dish in the environment within the chamber, a concentration of a specified reagent in the culture dish in the environment within the chamber.

18. The method of claim 13, wherein the least one chamber environment sensor includes at least one of a temperature sensor, a humidity sensor, a gas pressure sensor, a gas concentration sensor, a fluid concentration sensor, a fluid flow rate sensor, an assay sensor, a bioactivity electrical recording sensor, an orientation sensor, or a light sensor.

19. The method of claim 13, wherein the representation is captured by at least one image sensor, and wherein the representation of the culture includes at least one image of the culture.

20. The method of claim 13, further comprising:
analyzing the representation of the culture to detect a condition; and
outputting a notification that is indicative of the condition.
